(19) United States
(12) Patent Application Publication
(10) Pub. No.: US 2024/0382459 A1
(43) Pub. Date: Nov. 21, 2024

(54) MECHANISMS OF ANDROGEN RECEPTOR-CENTERED TRANSCRIPTIONAL NETWORKS IN REGULATING CD8+ T CELL EXHAUSTION AND THERAPEUTIC APPLICATIONS IN CANCER

(71) Applicant: Ohio State Innovation Foundation, Columbus, OH (US)

(72) Inventor: Zihai Li, Columbus, OH (US)

(21) Appl. No.: 18/662,816

(22) Filed: May 13, 2024

Related U.S. Application Data
(60) Provisional application No. 63/501,881, filed on May 12, 2023.

(51) Int. Cl. A61K 31/4166; A61P 35/00; C12Q 1/6874; C12Q 1/6886

(52) U.S. Cl. CPC A61K 31/4166; A61P 35/00; C12Q 1/6874; C12Q 1/6886; C12Q 2600/106; C12Q 2600/158

(57) ABSTRACT

Disclosed are methods for modulating androgen receptors in regulating T cell differentiation in cancer immunity, further reducing T cell exhaustion and/or treating sex-biased cancers through the administration of agents that inhibit androgen receptors. In some aspects the methods further comprise the detection of a sex biased cancer and/or T cell exhaustion via detection of GZMB+, TOX+ and/or T cell factor 1 (TCF1)+ T cells in the tumor microenvironment.

### II. CROSS-REFERENCE TO RELATED APPLICATIONS

This application claims the benefit of U.S. Provisional Application No. 63/501,881, filed on May 12, 2023, which is incorporated herein by reference in its entirety.

### I. STATEMENT OF GOVERNMENT SUPPORT

This invention was made with government support under grant/contract numbers R01 CA213290, R01 AI077283, R01 CA262069 awarded by the National Institutes of Health. The government has certain rights in the invention.

### III. BACKGROUND

Sex—as organized by the unequal composition and effects of sex chromosomes and gonadal hormones—is a biological variable with significant influence on immune function. However, mechanisms underlying sex-biased incidence and mortality of various cancers arising in non-reproductive organs remain elusive. While males are more prone to develop cancer in nearly every organ type, bladder cancer typifies this phenomenon with a striking 3- to 4-fold male-biased incidence globally that cannot be fully explained by established risk factors such as smoking, exposure to occupational hazards and urinary tract infections. What is needed are new ways to detect sex biases in cancers and treating said cancers.

### IV. SUMMARY

Disclosed are methods related to enhancing differentiation of CD8+ T cells to an effector state in a subject by inhibiting androgen receptor (AR) signaling in CD8+ T cells of the subject.

In one aspect, disclosed herein are methods for promoting/enhancing differentiation of T cells (such as, for example, CD8+ T cells) to an effector state in a subject comprising administering to the subject an inhibitor of androgen receptor (AR) (including, but not limited to genetic manipulation (such as, for example, anti-AR antibody, CRISPR/Cas9-mediated knockout of the AR gene in the CD8+ T cells) or androgen deprivation therapy) directed to an AR on the T cells of the subject. In one aspect, the androgen deprivation therapy comprises administration of Luteinizing Hormone-Releasing Hormone (LHRH) agonists comprising but not limited to leuprolide, goserelin, and triptorelin; the administration of androgen inhibitors comprising but not limited to bicalutamide, enzalutamide, apalutamide, flutamide, darolutamide, nilutamide, abiraterone, degarelix, relugolix, leuprolide, or goserelin; or administration of Androgen Synthesis Inhibitors comprising but not limited to abiraterone.

Also disclosed herein are methods for rescuing terminally exhausted T cells (including, but not limited to TOX+TCF1-CD8+ T cells) and/or progenitor exhausted T cells (TCF^{HI}) and driving said T cells to an effector-like state in a subject with a cancer comprises administering to the subject an inhibitor of androgen receptor (AR) (including, but not limited to genetic manipulation (such as, for example, anti-AR antibody, CRISPR/Cas9-mediated knockout of the AR gene in the CD8+ T cells) or androgen deprivation therapy). In one aspect, the androgen deprivation therapy comprises administration of Luteinizing Hormone-Releasing Hormone (LHRH) agonists comprising but not limited to leuprolide, goserelin, and triptorelin; the administration of androgen inhibitors comprising but not limited to bicalutamide, enzalutamide, apalutamide, flutamide, darolutamide, nilutamide, abiraterone, degarelix, relugolix, leuprolide, or goserelin; or administration of Androgen Synthesis Inhibitors comprising but not limited to abiraterone.

In one aspect, disclosed herein are methods for rescuing terminally exhausted T cells and/or progenitor exhausted T cells and driving said T cells to an effector-like state of any preceding aspect, further comprising monitoring the differentiation state of CD8+ T cells in the subject by flow cytometry analysis of T cell markers including GZMB, TOX, TCF1, and PD-1.

Also disclosed herein are methods for rescuing terminally exhausted T cells and/or progenitor exhausted T cells and driving said T cells to an effector-like state of any preceding aspect, wherein the cancer comprises T cell lymphoma, mycosis fungoides, Hodgkin's Disease, acute myeloid leukemia (AML), chronic myeloid leukemia (CML), brain cancer, nervous system cancer, head and neck cancer, renal cancer, lung cancers, neuroblastoma/glioblastoma, ovarian cancer, pancreatic cancer, skin cancer, hepatic cancer, melanoma, squamous cell carcinomas of the mouth, throat, larynx, and lung, cervical cancer, multiple myeloma, non-Hodgkin's lymphoma, breast cancer, genitourinary cancer, esophageal carcinoma, hematopoietic cancers, testicular cancer, colon cancer, or rectal cancer, sarcoma and gynecological malignancies. In some aspects, the cancer cells express AR receptor and/or exhibit AR-mediated immune modulation.

In one aspect, disclosed herein are methods for treating, inhibiting, reducing, decreasing, ameliorating, and/or preventing a cancer and/or metastasis in a subject, comprising: administering to the subject with the cancer a therapeutically effective amount of an androgen receptor (AR) inhibitor (including, but not limited to genetic manipulation (such as, for example, anti-AR antibody, CRISPR/Cas9-mediated knockout of the AR gene in the CD8+ T cells) or androgen deprivation therapy); wherein the AR inhibitor promotes differentiation of CD8+ T cells from a terminally exhausted state or progenitor exhausted T cell state to a granzyme B+ (GZMB+) effector-like state; and wherein the cancer is characterized by AR-dependent modulation of immune responses. In one aspect, the androgen deprivation therapy comprises administration of Luteinizing Hormone-Releasing Hormone (LHRH) agonists comprising but not limited to leuprolide, goserelin, and triptorelin; the administration of androgen inhibitors comprising but not limited to bicalutamide, enzalutamide, apalutamide, flutamide, darolutamide, nilutamide, abiraterone, degarelix, relugolix, leuprolide, or goserelin; or administration of Androgen Synthesis Inhibitors comprising but not limited to abiraterone.

Also disclosed herein are methods for treating, inhibiting, reducing, decreasing, ameliorating, and/or preventing a cancer and/or metastasis of any preceding aspect, wherein the modulation of immune responses further comprises monitoring differentiation state of CD8+ T cells in the subject by flow cytometry analysis of T cell markers including GZMB, TOX, TCF1, and PD-1.

In one aspect, disclosed herein are methods for treating, inhibiting, reducing, decreasing, ameliorating, and/or preventing a cancer and/or metastasis of any preceding aspect further comprising subjecting the subject to conventional cancer therapy, wherein the conventional cancer therapy comprises including but not limited to surgery, chemotherapy, or radiation therapy.

Also disclosed herein are methods for treating a subject with a cancer comprising a) obtaining a biological sample from the subject; b) measuring the level of androgen receptor (AR) expression or activity in CD8+ T cells; wherein an increase in the level of AR expression or activity relative to a control indicates suitability for AR inhibitor therapy; and c) administering an AR inhibitor (including, but not limited to genetic manipulation (such as, for example, anti-AR antibody, CRISPR/Cas9-mediated knockout of the AR gene in the CD8+ T cells) or androgen deprivation therapy) when the subject has an increase in the level of AR expression or activity. In one aspect, the androgen deprivation therapy comprises administration of Luteinizing Hormone-Releasing Hormone (LHRH) agonists comprising but not limited to leuprolide, goserelin, and triptorelin; the administration of androgen inhibitors comprising but not limited to bicalutamide, enzalutamide, apalutamide, flutamide, darolutamide, nilutamide, abiraterone, degarelix, relugolix, leuprolide, or goserelin; or administration of Androgen Synthesis Inhibitors comprising but not limited to abiraterone.

Also disclosed herein are methods for treating, inhibiting, reducing, decreasing, ameliorating, and/or preventing a cancer and/or metastasis of any preceding aspect, wherein the cancer comprises T cell lymphoma, mycosis fungoides, Hodgkin's Disease, acute myeloid leukemia (AML), chronic myeloid leukemia (CML), brain cancer, nervous system cancer, head and neck cancer, renal cancer, lung cancers, neuroblastoma/glioblastoma, ovarian cancer, pancreatic cancer, skin cancer, hepatic cancer, melanoma, squamous cell carcinomas of the mouth, throat, larynx, and lung, cervical cancer, multiple myeloma, non-Hodgkin's lymphoma, breast cancer, genitourinary cancer, esophageal carcinoma, hematopoietic cancers, testicular cancer, colon cancer, or rectal cancer, sarcoma and gynecological malignancies. In some aspects, the cancer cells express AR receptor and/or exhibit AR-mediated immune modulation.

### V. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, and IF show CD8⁺ T cell immunity mediates sex differences in murine bladder cancer aggression. FIG. 1A shows BBN-induced carcinogenesis model. Mice were exposed ad libitum to 0.1% BBN in drinking water for the first 14 weeks to induce bladder cancer formation and then monitored for a total of 302 days. Percent survival, genotype/sex of each experimental group, and p values from the log rank test are shown. N=13-20 mice per group. FIG. 1B shows MB49 tumor growth in mice with indicated genotypes. FIG. 1C shows antibody-mediated depletion of CD4⁺ and/or CD8⁺ cells in mice challenged with MB49. FIG. 1D shows RT-PCR for qualitative detection of β-actin and Y chromosome encoded Sry gene transcripts from MB49 cells. DNA extracted from tails of male and female mice are included as controls. FIG. 1E shows a diagram representation of Four Core Genotype (FCG) mouse model. BKL171 was generated from a BBN-induced bladder tumor of a XXM FCG mouse. FIG. 1F shows BKL171 tumor growth in mice with indicated genotypes after subcutaneous implantation. Mean tumor area (mm²)±SEM are reported, with statistical significance determined using the repeated measures two-way ANOVA. n=4-10 mice per group. M, male; F, female; Ab, antibody; ns, not significant. *p≤0.05, **p≤0.01, ***p≤ 0.001, ****p≤0.0001. p-values in A and F were corrected for multiple testing using the Bonferroni procedure.

FIGS. 2A and 2B show superior effector function of tumor infiltrating T cells in female mice underlies sex-biased cancer outcomes. FIG. 2A shows flow cytometric analysis of IFNγ, TNFα and GZMB expression in CD8⁺ T cells from the spleens and tumors (TILs) of male and female mice 9 days post subcutaneous MB49 challenge. Cells were stimulated ex vivo with 50 ng/mL PMA, 1 μg/mL Ionomycin and 1× Brefeldin A for 2 hours. Statistical significance was determined using Student's t test. FIG. 2B shows the impact of donor and recipient sex on the adoptive transfer therapy of T cells. MB49 tumor growth in Tcrb/Tcrd KO mice post adoptive transfer on Day −1 with 5×10⁵ CD8⁺ T cells, which were prepared separately from the draining lymph nodes of Day 14 MB49 tumor in WT mice. Mean tumor area (mm²)±SEM are reported, with statistical significance determined using the repeated measures two-way ANOVA. n=4-10 mice per group. *p≤0.05, **p≤0.01, ***p≤0.001.

FIGS. 3A, 3B, 3C, 3D, 3E, and 3F show sex differences in CD8⁺ T cell immunity at single-cell resolution in the tumor microenvironment. FIG. 3A shows Uniform Manifold Approximation and Projection (UMAP) of 26,698 CD8⁺ TIL (3 female mice—9,955; 3 male mice—16,743) scRNA-seq profiles, colored by cluster. Cells were sorted by FACS from Day 10 MB49 tumors. 9,955 cells are shown here for each sex for the purpose of easier visualization. Enclosed clusters 1, 2, 7 and 9 show male-biased frequencies as outlined in (3B). FIG. 3C shows a summary of key CD8⁺ T cell genes that show sex-biased expression level in each cell cluster. Genes with Bonferroni-adjusted p-values≤0.05 from the sex-based differential expression analyses using Wilcoxon rank-sum test were considered male (blue) or female (purple) biased depending on the directionality of their average log fold change. FIG. 3D shows enrichment pattern of published PE and effector-like CD8⁺ T cell signatures. FIG. 3E shows ontogeny inference. Slingshot algorithm was applied to order single cells in clusters 1, 2, 6, 7, 9 and 10 in pseudotime. Tcf7, Cd44, Gzmb, Prf1, Havcr2 and Pdcd1 expression levels in these clusters are indicated. FIG. 3F shows quantification of pseudotime trajectories of male and female Tcf7+CD8⁺ TILs from E. Statistical significance was determined using Wilcoxon rank-sum test. ****p≤0.0001.

FIGS. 4A, 4B, 4C, 4D, and 4E show male-biased CD8⁺ progenitor exhausted T cell frequency in the tumor microenvironment. FIG. 4A shows a UMAP of CD8⁺ TILs from MB49 tumors as assessed by spectral flow cytometry at indicated time points, colored by cluster. Enclosed cluster 2 shows a male-biased frequency as indicated. FIG. 4B show indicated protein expression on UMAP. FIG. 4C shows the percentage of the CD8⁺ TILs at indicated or all combined time points using the PE score, which was generated by UCell based on protein-level expression of (TCF1, SLAMF6, BCL2, CD44, CD69) or (CD62L, PD1, LAG3, TIM3, CTLA4, TOX) that are positively and negatively associated with PE cells, respectively. FIG. 4D shows MB49 growth in Rag2 KO mice that were adoptively transferred with 1.75×103 TIM3-SLAMF6+CD8+ T cells from Day 12 MB49 tumors of WT mice. Tumor weights from Day 14 are reported. FIG. 4E shows flow cytometric analysis of TIM3 and TCF1 expression in donor TILs. Statistical significance for % PE cells was determined by Wilcoxon rank-sum test. Remaining statistical significance was determined by Student's t test. *p≤0.05, **p≤0.01, ***p≤0.001. p-values in 4D and 4E were corrected for multiple testing using the Bonferroni procedure.

FIGS. 5A, 5B, 5C, 5D, 5E, 5F, 5G, 5H, 5I, and 5J show T cell intrinsic AR signaling contributes to male bias in tumor growth and accumulation of CD8⁺ progenitor exhausted T cells. MB49 growth and circulating free testosterone levels in (5A) Four Core Genotype or (5B) WT mice three weeks post sham or surgical castration (cast). FIG. 5C shows PCA of spectral flow cytometry data on CD8⁺ TILs and (5D) percentage of CD8⁺PE T cells from 5B. FIG. 5E shows MB49 growth in castrated WT mice after intraperitoneal injection with IgG control or anti-mouse CD8 antibodies. dep1, Depletion. FIG. 5F shows immunoblot of AR and β-actin in stimulated CD8⁺ T cells from Ar^{fl/(fl)} control or E8iCre-Ar^{fl/(fl)} mouse spleens. LNCaP lysates were used as the positive AR control. Rep, replicate. FIG. 5G shows MB49 growth in Ar^{fl/(fl)} or E8iCre-Ar^{fl/(fl)} mice. FIG. 5H shows the percentage of male CD8⁺ PE T cells from 5G. FIG. 5I shows the percentage of male TOX⁺ TCF1⁻ cells from 5G. FIG. 5J shows MB49 growth in WT mice after daily treatment of vehicle or enzalutamide (enz) starting on Day 5. For tumor growths, mean area (mm²)±SEM are reported, with statistical significance determined using the repeated measures two-way ANOVA. n=6-11 mice per group. Remaining statistical significance was determined by Student's t test. *p≤0.05, **p≤ 0.01, ***p≤0.001, ****p≤0.0001. p-values in 5A, 5B, 5D, 5E, 5G, and 5J were corrected for multiple testing using the Bonferroni procedure.

FIGS. 6A, 6B, 6C, 6D, 6E, 6F, and 6G show AR signaling in CD8⁺ progenitor exhausted T cells. FIG. 6A shows enrichment of androgen response (red) and type I IFN (blue) signatures in CD8⁺ TILs from Day 10 MB49 tumors. FIGS. 6B and 6C show pseudotime analysis of lymphocytic choriomeningitis virus specific CD8⁺ T cells, as colored by the relative enrichment of androgen response signature. Boxplots indicate expression of androgen-responsive genes in effector-like (“Eff”) and progenitor exhausted (“Ex”) cells. FIG. 6D shows Log₂-transformed ratio of ATAC-seq signals for androgen-responsive genes in progenitor (SLAMF6⁺TIM3⁻) over terminally (SLAMF6⁻TIM3⁺) exhausted CD8⁺ T cells. FIG. 6E shows multiplexed immunofluorescent analysis of nuclei, CD3, CD8, TIM3, TCF1 and AR in BBN-induced bladder tumors collected from male mice at the time of sacrifice for morbidity. Scale bar, 1000 μm (top left), 100 μm (top right), 15 μm (bottom). FIG. 6F shows quantification of CD8⁺ T cells in normal bladder versus tumor (left) and frequency of AR-expressing CD8⁺ T cell in the indicated cell subsets (right). FIG. 6G shows qPCR analysis of FACS-sorted CD8⁺ T cell subsets (red box) from Day 14 MB49 tumors. Statistical significance was determined by Student's t test. *p≤0.05, **p≤0.01, ***p≤0.001, ****p≤0.0001. p-values in F (right) were corrected for multiple testing using the Bonferroni procedure.

FIGS. 7A, 7B, 7C, 7D, 7E, 7F, and 7G show AR modulation of Tcf7 and its transcriptional network. FIG. 7A shows the workflow of Integrated Cell-type-specific Regulon Inference Server from single-cell RNA-Seq (IRIS3). See methods for details. FIG. 7B shows sex differences in Tcf7 centered regulon in the CD8⁺PE TILs. Enclosed transcription factors have AREs within their respective promoters. FIG. 7C shows the correlation in expression between indicated transcription factors and Tcf7 in male-biased scRNA-seq clusters. FIG. 7D shows qPCR analysis of indicated genes in effector memory (“EFF”), central memory (“CM”) or naïve CD8⁺ T cells. Naïve CD8⁺ T cells were stimulated with testosterone, type I IFN or both for 6 hours. Blue, Male; Pink, Female. FIG. 7E shows luciferase reporter assay. Renilla and Red Firefly luciferase activity, the latter of which was regulated by a 1 kb long WT or mutant Tcf7 promoter sequence lacking putative AREs (FIG. 13A), were simultaneously measured in control or EGFP-C1-AR overexpressing HEK293FT post 24 h stimulation with 500 ng/mL testosterone (T) or 50 nM DHT. FIG. 7F shows AR ChIP-qPCR of EV- and AR-Jurkats post 24 h of 100 nM DHT stimulation compared to vehicle control. FIG. 7G shows a schematic representation of sex differences CD8⁺ T cell fate in the tumor microenvironment. Statistical significance was determined by Student's t test. *p≤0.05, **p≤0.01, ***p≤0.001.

FIGS. 8A, 8B, 8C, 8D, 8E, and 8F show T cell numbers are comparable between male and female MB49-bearing mice. CD45⁺, CD3⁺CD8⁺ and CD3⁺CD4⁺ immune cell frequency and absolute number—as assessed by flow cytometry—in Day 9 MB49 tumors (8A to 8C) or spleens (8D to 8F) are indicated in left and right y axes of each graph, respectively. Box height represents the mean of all shown biological replicates. Blue, male; Pink, female.

FIGS. 9A, 9B, and 9C shows the characterization of CD8⁺ TILs from Day 10 MB49 tumors by single cell RNA sequencing analysis. FIG. 9A shows a heatmap of key CD8⁺ T cell gene expression for each cell cluster. These genes show significant sex-biased expression as indicated in FIG. 3C. Genes with Bonferroni-adjusted p-values≤0.05 from the differential expression analyses using Wilcoxon rank-sum test were considered enriched (red) or depleted (blue) depending on the directionality of their average log fold change. White color indicates no cluster specific gene enrichment. FIG. 9B shows the expression of indicated genes in individual cells. FIG. 9C shows the expression of Tcf7 (left) and Gzmb (right) in clusters 1, 2, 6, 7, 9 and 10 across pseudotime in males (solid) versus females (dotted).

FIGS. 10A, 10b, 10c, 10d, 10E, 10F, 10G, 10H, and 10I show characterization of CD8⁺ progenitor exhausted T cells from male versus female tumors. FIG. 10A shows cluster frequencies (lines) in males (blue) and females (pink), along with 95% confidence intervals (vertical bars). See FIG. 4A. FIG. 10B shows a heatmap of relative protein expression of markers in (10A). Blue, low expression; Red, high expression. FIG. 10C shows flow cytometric analysis of TCF1 and TOX expression in male and female CD44+CD62L⁻CD8⁺ T cells at indicated time points. FIG. 10D shows MC38 and B16 tumor growth in WT mice. Mean tumor area (mm²)±SEM are reported, with statistical significance determined using the repeated measures two-way ANOVA. FIG. 10E shows the frequency of indicated PE CD8⁺ T cells in MC38 and B16-F10 tumors. Blue, male; Pink, female. FIG. 10F shows enrichment analysis of the PE signature in minimum spanning tree (MST) based clusters from the spectral flow cytometry data. Dotted line represents the 75th percentile of PE scores across all cells. FIG. 10G shows representative flow plot of SLAMF6 and TCF1 co-expression in CD44⁺CD62L⁻TIM3^{-/+}CD8⁺ TILs. FIG. 10H shows the percentage of SLAMF6⁺TIM3⁻ TILs in MB49 tumors. FIG. 10I shows representative plots of flow cytometric analysis of IFNγ and TNFα expression in donor TILs post ex vivo stimulation with 50 ng/mL PMA, 1 μg/mL Ionomycin and 1× Brefeldin A for 2 hours. See FIG. 4D. Statistical significance for C, E and H was determined by Student's t test. *p≤0.05, **p≤0.01, ****p≤0.0001.

FIGS. 11A and 11B show male-biased CD8⁺ T cell exhaustion in human cancer. UMAP of CD8⁺ TILs from (11A) basal cell carcinoma (BCC) prior to immunotherapy or (11B) treatment-naïve non-small cell lung cancer (NSCLC). T cell differentiation states were annotated the same way as published and stratified based on patients' sex. Solid black lines enclose exhausted clusters that show male-biased frequency. FIG. 11A shows Act, activated; Eff, effector; Ex, exhausted; Ex_act, exhausted/activated; Mem, memory. FIG. 11B shows C1-LEF1=naïve, [C2-CD28, C4-GZMK, C5-ZNF683]=intermediate between naïve and effector, C3-CX3CR1=effector, C6-LAYN=exhausted, C7-SLC4A10=mucosal-associated invariant T (MAIT) cells. Stacked bar charts show frequency of indicated clusters in women and men.

FIGS. 12A and 12B show AR signaling in CD8⁺ progenitor exhausted T cells. FIG. 12A shows a UMAP of scRNA-seq profiles of CD8⁺ TILs from BBN-induced urothelial carcinoma, as colored by the relative enrichment of androgen response signature. PE T cells are enclosed. FIG. 12B shows the correlation between androgen and type I IFN signatures in MB49 CD8⁺ TILs, as well as human bladder cancer (BLCA), basal cell carcinoma (BCC), and non-small cell lung cancer (NSCLC), in females and males.

FIGS. 13A, 13B, and 13C show the direct regulation of Tcf7 by Androgen Receptor. FIG. 13A shows a list of AREs in human and mouse Tcf7 promoter. Conserved AR binding motif was scanned through 1 kb upstream of Tcf7 transcriptional start site via Motif Alignment and Search Tool (MAST) at a positional p value<0.005. Shown are hARE1 binding site: CGGGCTGCAGGTTCT (SEQ ID NO: 21), hARE2 binding site AACGCTGCCGGTTCC (SEQ ID NO: 22), hARE3 binding site AGCACAGGGCGCATT (SEQ ID NO: 23), hARE4 binding site GGAAGAGCGAGCCCT (SEQ ID NO: 24), mARE1 binding site TGGACAGCAGGTTCT (SEQ ID NO: 25), mARE2 binding site ACAACAGGCAGGAGC (SEQ ID NO: 26), mARE3 binding site AGGAAATGGAGTTCC (SEQ ID NO: 27), mARE4 binding site TGCCTTTGATGTTCC (SEQ ID NO: 28), and mARE5 binding site GGACCTGGCAGAGCT (SEQ ID NO: 29). FIGS. 13B and 13C show experimental analysis of predicted AREs. Red Firefly luciferase activity, as regulated by a 1 kb long human WT or mutant Tcf7 promoter sequence lacking putative AREs, was measured in EGFP-C1-AR overexpressing HEK293FT cells post 24 h stimulation with 500 ng/mL testosterone or vehicle control. EGFP was assessed by flow cytometry to ensure comparable transfection efficiency. Statistical significance was determined by Student's t test. *p≤0.05, **p≤ 0.01, ***p≤0.001, ****p≤0.0001, ns=not significant.

FIGS. 14A and 14B show the generation of AR overexpressing Jurkats. FIG. 14A shows qPCR/western blot analysis of AR expression in HEK293FT, LNCaP and WT, empty vector (EV) or AR-overexpressing (AR) Jurkats. β-actin served as a loading control. FIG. 14B shows the proliferation of EV- and AR-Jurkats over 9 days during stimulation with vehicle control, 100 nM testosterone (T) or 100 nM dihydrotestosterone (DHT) at Days 0, 3 and 6. n=5 biological replicates. Data are mean±SEM. Statistical significance was determined by Student's t test. **p≤0.01, ****p≤0.0001.

FIGS. 15A, 15B, 15C, and 15D show ablation of Androgen Receptor (AR) Signaling Attenuates Tumor Aggressiveness in Murine Bladder Cancer. FIG. 15A shows MB49 tumor growth in male wild-type CD57B/6 mice having undergone surgical castration (red, n=6) or sham surgery (blue, n=7). FIG. 15B shows MB49 tumor growth in male AR^{floxed} (blue, WT, n=5) or E8iCre- AR^{floxed} (red, AR KO, n=5) mice. FIG. 15C shows mean fluorescence intensity (MFI) of TOX from tumor infiltrating CD8+ T cells in mice from experiments in panels A & B. FIG. 15D shows the frequency of TOX+TCF1− terminally exhausted tumor infiltrating CD8+ T cells as a percentage of Cd44+CD62L− CD8+ T cells from experiments in FIGS. 15A & 15B. Mean tumor volumes±SEM are reported, with statistical significance determined by redetermined by repeated measures two-way ANOVA. *P<0.05, **P<0.01, ***P<0.001 and ****P<0.0001.

FIGS. 16A, 16B, and 16C show AR maintains TCF1^{high} stem-like states and represses effector differentiation and function in murine CD8+ T cells, which is reversed by AR knockout. FIG. 16A shows flow cytometry characterization of splenic CD8+ T cells from non-tumor-bearing wild-type (AR^{floxed}, grey, n=6) or CD8-specific AR knockout (E8iCre- ARfloxed, red, n=4) mice demonstrates lower frequency of TCF1^{high} central memory cells in AR KO CD8+ T cells. FIG. 16B shows CRISPR-mediated AR knockout in splenic CD8+ T cells from non-tumor bearing Pmel mice activated in vitro with gp100 peptide shows lower frequency of CD44+CD62L+ central memory CD8+ T cells and higher frequency of CD25+CD69+ effector-like cells in AR KO vs scrambled guide-RNA control conditions. FIG. 16C shows CD8+ T cells with CRISPR-mediated AR KO have higher IFNγ expression upon PMA/inomycin+BFA stimulation. Statistical significance in FIG. 16A determined by Student's t test. *P≤0.05.

FIGS. 17A, 17B, 17C, and 17D show CUT&Tag assay identifies AR genomic binding loci in murine CD8+ TILs. FIG. 17A shows schematic of CUT&Tag assay to identify transcriptional targets of AR in murine MB49 tumor infiltrating CD8+ T cells. FIG. 17B shows AR and H3K4me3 binding site gene distances from transcriptional start sites (TSS). FIG. 17C shows functional enrichment analysis of AR-regulated genes, top 10 gene ontology terms presented. FIG. 17D shows gene browser tracks showing AR, H3K4me3 and IgG control readouts across Tox and Tcf7 gene loci.

FIGS. 18A, 18B, 18C, and 18D show Androgen deprivation therapy alters CD8+ T cell exhaustion programs in men with castrate-sensitive prostate cancer FIG. 18A shows high dimensional flow cytometry using a 23 marker T cell exhaustion panel on peripheral blood mononuclear cells drawn at month 0 (baseline) before ADT initiation or 1, 3, 6 or 12 months post-initiation of ADT in 18 men with newly diagnosed localized or metastatic castrate-sensitive prostate cancer; data presented in uniform manifold approximation and projection (UMAP) space. Red circles indicate manual cluster annotations. FIG. 18A shows frequency of TCF1^{high} PD-1^{intermediate} progenitor exhausted cells, as a percentage of CD45RA-CD8+ T cells. FIG. 18C shows frequency of EOMES^{high} T-bet^{low} TOX^{high} PD-1^{high} terminal exhausted-like cells, as a percentage of CD45RA-CD8+ T cells. FIG. 18D shows frequency of EOMES^{low} T-bet^{high} TOX^{intermediate} PD-1^{high} GZMB^{high} effector-like cells, as a percentage of CD45RA-CD8+ T cells. Statistical significance in FIGS. 18B-D determine by a mixed effects model for repeated measures data; error bars represent±SEM. *P≤0.05 and ***P≤0.001. P values were corrected for multiple hypothesis testing using the Dunnett method.

FIGS. 19A, 19B, 19C, 19D and 19E show androgen receptor blockade of in vitro expanded CD8+ bladder cancer TILs promotes effector differentiation and function. FIG. 19A shows effect of DHT treatment on CD8+GZMB^{high} population in ex vivo expanded TILs from female patients with bladder cancer. FIG. 19B shows effect of AR blockade with 50 uM enzalutamide in the presence of 10 nM DHT on the frequency of CD8+GZMB^{high} TILs compared to vehicle control. FIG. 19C shows frequency of PD-1^{high}TOX^{high}CD8+ TILs with varying doses of enzalutamide. FIG. 19D shows IFNγ production in CD8+ TILS after PMA/ionomycin/golgi block stimulation+/−enzalutamide. FIG. 19E shows median fluorescence intensity (MFI) of IFN-gamma within CD8+GZMB^{high} population. Statistical significance in FIGS. 19A-C determined by repeated measures ANOVA. P values were corrected for multiple hypothesis testing using the Dunnett method. Statistical significance in FIG. 19E determined by Student's paired t test. *P≤0.05.

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHOD OF TREATING CANCER

Contribution of CD8⁺ T cell immunity to sex differences in tumor development and progression remains unclear. CD8⁺ T cells have an enormous potential to eliminate malignant cells based on their recognition of antigens distinct from those of normal cells by abundance or molecular structure. However, they often become exhausted in the tumor microenvironment upon persistent antigen stimulation, which is characterized by progressive loss of effector and proliferative potential, sustained expression of immune checkpoint receptors (e.g., PD1, TIM3, LAG3) and a distinct transcriptional and epigenetic landscape. Importantly, exhausted CD8⁺ T cells are phenotypically heterogeneous with varying degrees of functional defects. Defined by their relatively stem-like genetic profile with low to no expression of immune checkpoint receptors, CD8⁺ progenitor exhausted (PE) T cells have the potential to proliferate and give rise to effector-like CD8⁺ T cells, particularly in response to anti-PD1 therapy. By contrast, terminally exhausted CD8⁺ T cells demonstrate high cytotoxic function, but they produce little or no effector cytokines upon re-stimulation, lack durability and are subject to rapid cell death mediated clearance.

The tumor microenvironment contains cellular and molecular entities including immunosuppressive myeloid cells, CD4⁺ Foxp3⁺ regulatory T cells, inhibitory cytokines (e.g., IL-10/TGFβ), immune checkpoint receptors and ligands (e.g., PD1 and PD-L1), and metabolic challenges (e.g., local nutrient competition), that collectively promote T cell dysfunction. Pertinent contributions by sex hormones in T cell programming in the tumor microenvironment are poorly understood. Here, we determine sex differences in CD8⁺ T cell-mediated anti-tumor immunity and elucidate an underlying androgen-dependent mechanism of T cell exhaustion.

Accordingly, disclosed herein are methods of treating, decreasing, reducing, inhibiting, ameliorating, and/or preventing a cancer and/or metastasis (excluding prostate cancer) in a subject (such as a male subject) comprising administering to the subject (such as a male subject) an agent that reduces androgen receptor (AR) signaling (including, but not limited to a genetic manipulation (such as, for example, anti-AR antibody, CRISPR/Cas9-mediated knockout of the AR gene in the CD8+ T cells) and/or androgen deprivation therapy including, but not limited to the administration of small molecules, siRNA, shRNA, RNAi, anti-sense oligonucleotide, peptide, protein, or antibody that inhibits AR signaling). In one aspect, the androgen deprivation therapy comprises administration of Luteinizing Hormone-Releasing Hormone (LHRH) agonists comprising but not limited to leuprolide, goserelin, and triptorelin; the administration of androgen inhibitors comprising but not limited to bicalutamide, enzalutamide, apalutamide, flutamide, darolutamide, nilutamide, abiraterone, degarelix, relugolix, leuprolide, or goserelin; or administration of Androgen Synthesis Inhibitors comprising but not limited to abiraterone.

It is understood and herein contemplated that not all males with cancer have a cancer with a sex bias and some females can experience a cancer with a sex bias. Thus, detecting the presence of a sex bias can be advantageous in knowing which subjects should receive treatment. As shown herein, thymocyte selection associated high mobility group box (TOX)+ and/or T cell factor 1 (TCF1)+ T cells have an exhausted phenotype and are abundant in cancers showing a sex bias. Thus, the detection of TOX+ and/or TCF1+ T cells indicates the presence of a sex bias. Accordingly, disclosed herein are methods of treating, decreasing, reducing, inhibiting, ameliorating, and/or preventing a cancer and/or metastasis in a subject (such as a male subject) said method comprising a) assaying the T cells in the tumor microenvironment for being TOX+ and/or TCF1+ T cells; wherein the presence of TOX+ and/or TCF1+ T cells indicates a male sex bias; and b) administering to the subject an agent the reduces androgen receptor (AR) signaling (including, but not limited to a small molecule, siRNA, shRNA, RNAi, anti-sense oligonucleotide, peptide, protein, or antibody that inhibits AR signaling) when TOX+ and/or TCF1+ T cells are detected thereby reducing AR transcriptional regulation and exhaustion of T cells.

Detection of TOX+ and/or TCF1+ T cells can be achieved by any means known in the art, including, but not limited to immunofluorescence, flow cytometry, RNA sequencing, microarrays, enzyme linked immunoassay (ELISA), enzyme linked immunospot assay (ELIspot), reporter assays (such as, for example, a luciferase reporter assay), and/or polymerase chain reaction (PCR).

As shown herein that use of AR deprivation or targeted disruption of AR can drive the differentiation of T cells (including, but not limited to progenitor exhausted T cells and terminally exhausted T cells) to effector cells. Accordingly, in one aspect, disclosed herein are methods for treating, inhibiting, reducing, decreasing, ameliorating, and/or preventing a cancer and/or metastasis in a subject, comprising: administering to the subject with the cancer a therapeutically effective amount of an androgen receptor (AR) inhibitor (including, but not limited to genetic manipulation (such as, for example, anti-AR antibody, CRISPR/Cas9-mediated knockout of the AR gene in the CD8+ T cells) or androgen deprivation therapy); wherein the AR inhibitor promotes differentiation of CD8+ T cells from a terminally exhausted state or progenitor exhausted T cell state to a granzyme B+ (GZMB+) effector-like state; and wherein the cancer is characterized by AR-dependent modulation of immune responses. In one aspect, the androgen deprivation therapy comprises administration of Luteinizing Hormone-Releasing Hormone (LHRH) agonists comprising but not limited to leuprolide, goserelin, and triptorelin; the administration of androgen inhibitors comprising but not limited to bicalutamide, enzalutamide, apalutamide, flutamide, darolutamide, nilutamide, abiraterone, degarelix, relugolix, leuprolide, or goserelin; or administration of Androgen Synthesis Inhibitors comprising but not limited to abiraterone.

In some aspects, the methods of treatment can be preceded by detection or diagnosis or a sex biased cancer. Thus, in one aspect, disclosed herein are methods for treating a subject with a cancer comprising a) obtaining a biological sample from the subject; b) measuring the level of androgen receptor (AR) expression or activity in CD8+ T cells; wherein an increase in the level of AR expression or activity relative to a control indicates suitability for AR inhibitor therapy; and c) administering an AR inhibitor (including, but not limited to genetic manipulation (such as, for example, anti-AR antibody, CRISPR/Cas9-mediated knockout of the AR gene in the CD8+ T cells) or androgen deprivation therapy) when the subject has an increase in the level of AR expression or activity. In one aspect, the androgen deprivation therapy comprises administration of Luteinizing Hormone-Releasing Hormone (LHRH) agonists comprising but not limited to leuprolide, goserelin, and triptorelin; the administration of androgen inhibitors comprising but not limited to bicalutamide, enzalutamide, apalutamide, flutamide, darolutamide, nilutamide, abiraterone, degarelix, relugolix, leuprolide, or goserelin; or administration of Androgen Synthesis Inhibitors comprising but not limited to abiraterone.

The disclosed methods can be used to treat any disease where uncontrolled cellular proliferation occurs such as cancers excluding prostate cancer. A representative but non-limiting list of cancers that the disclosed methods can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers. Thus disclosed herein are methods of treating, decreasing, reducing, inhibiting, ameliorating, and/or preventing a cancer and/or metastasis wherein the cancer comprises T cell lymphoma; mycosis fungoides; Hodgkin's Disease; acute myeloid leukemia (AML); chronic myeloid leukemia (CML); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; skin cancer; hepatic cancer; melanoma; squamous cell carcinomas of the mouth, throat, larynx, and lung; cervical cancer; cervical carcinoma; breast cancer, triple negative breast cancer; genitourinary cancer; pulmonary cancer; esophageal carcinoma; head and neck carcinoma; large bowel cancer; hematopoietic cancers; testicular cancer; and colon and rectal cancer. In one aspect, the cancer is not prostate cancer.

In one aspect, the treatment of the cancer can include administration of an agent that inhibits, disrupts, reduces, and/or decreases signaling by the androgen receptor. Such inhibitors can include small molecules, functional nucleic acids (for example, antisense oligonucleotides, siRNA, shRNA, or RNAi), peptides, proteins, and/or antibodies (including diabodies, nanobodies, scFv, sFv, and functional antibody fragments). Thus, disclosed herein are methods of treating, inhibiting, reducing, decreasing, ameliorating, and/or preventing a cancer and/or metastasis (excluding prostate cancer) in a subject comprising administering to the subject an agent the reduces androgen receptor (AR) signaling (such as, for example, a small molecule, siRNA, shRNA, RNAi, peptide, protein, or antibody that inhibits AR signaling) including, but not limited to bicalutamide, enzalutamide, apalutamide, flutamide, darolutamide, nilutamide, abiraterone, degarelix, relugolix, leuprolide, or goserelin.

It is understood and herein contemplated that the disclosed methods for treating, inhibiting, reducing, decreasing, ameliorating, and/or preventing a cancer and/or metastasis of can further comprising subjecting the subject to conventional cancer therapy, wherein the conventional cancer therapy comprises including but not limited to surgery, chemotherapy, immunotherapy (such as, for example, the administration of antibodies, checkpoint inhibitors, chimeric antigen receptor (CAR) T cells, CAR natural killer (NK) cells, CAR macrophage, CAR B cells, tumor infiltrating lymphocytes, and/or radiation therapy.

In one aspect it is understood that the disclosed treatment regimens can used alone or in combination with any anti-cancer therapy known in the art including, but not limited to CAR-T (Chimeric Antigen Receptor T-Cell) treatments. The CAR-T cell treatments include:

Multi-Antigen Targeting CAR-T Cells: These CAR-T cells are engineered to recognize multiple antigens simultaneously, enhancing their ability to eradicate tumor cells and reduce the risk of antigen-negative tumor escape, which can lead to treatment failure in single-target CAR-T therapies.

Switchable CAR-T Therapy: Uses CAR-T cells engineered to be activated or deactivated by specific, non-toxic small molecules. This switch mechanism allows for precise control over the CAR-T cells' activity, potentially improving safety and reducing side effects such as cytokine release syndrome.

Memory-Like CAR-T Cells: Focusing on longevity and sustained response, these CAR-T cells are engineered to exhibit memory-like properties, allowing them to persist in the body and provide long-term surveillance against cancer recurrence, much like natural memory T cells.

Tissue-Specific Homing CAR-T Cells: These are designed with receptors that enable them to specifically home in on the tissues where the tumor is located, potentially increasing the efficacy and minimizing off-tumor toxicity by limiting their activity to the target tissue site.

Next-Generation Signaling CAR-T Cells: Enhancing the intracellular signaling domains within CAR constructs can lead to more robust and sustained T-cell activation. By incorporating novel co-stimulatory domains or modifying existing ones, these CAR-T cells can be tailored for greater anti-tumor efficacy.

CAR-T Cells with Integrated Checkpoint Inhibition: These CAR-T cells include genetic modifications that allow them to counteract the immune-suppressive tumor microenvironment. By blocking inhibitory signals (like PD-1 or CTLA-4) within the CAR structure itself, these cells can sustain their activity in hostile tumor conditions.

Light-Activated CAR-T Cells: Utilizing optogenetics, these CAR-T cells can be controlled through exposure to specific wavelengths of light, allowing precise spatial and temporal control of their therapeutic activity, which could significantly reduce collateral damage to healthy cells.

Metabolically Enhanced CAR-T Cells: These CAR-T cells are engineered to maintain functionality in the metabolically challenging environment of solid tumors, which often exhibit low nutrients and high acidity. Modifications might include altering metabolic pathways to sustain cell viability and function under such conditions.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R—CHOP, R—CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizurnab, avelumab, durvaluimab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-dornain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS118, MGD013, and Immutep).

### C. METHODS OF REDUCING T CELL EXHAUSTION

It is understood and herein contemplated that the disclosed treatment methods work by inhibiting the androgen receptor signaling the results in T cell exhaustion. These T cells are identified by being thymocyte selection associated high mobility group box (TOX)+ and/or T cell factor 1 (TCF1)+ T cells. Thus, the methods employed to treat cancer can also be used to reduce T cell exhaustion in the tumor microenvironment. Accordingly, in one aspect, disclosed herein are methods of reducing, inhibiting, decreasing, treating, and/or preventing T cell exhaustion in the tumor microenvironment of a subject with a cancer the method comprising administering to the subject (such as a male subject) an agent that reduces androgen receptor (AR) signaling (including, but not limited to a small molecule, siRNA, shRNA, RNAi, anti-sense oligonucleotide, peptide, protein, or antibody that inhibits AR signaling). In one aspect, the T cells the T cells in the tumor microenvironment before treatment are TOX+ and/or TCF1+.

Similarly, disclosed herein are methods for rescuing terminally exhausted T cells (including, but not limited to TOX+TCF1− CD8+ T cells) and/or progenitor exhausted T cells (TCF^{HI}) and driving said T cells to an effector-like state in a subject with a cancer comprises administering to the subject an inhibitor of androgen receptor (AR) (including, but not limited to genetic manipulation (such as, for example, anti-AR antibody, CRISPR/Cas9-mediated knockout of the AR gene in the CD8+ T cells) or androgen deprivation therapy). Also disclosed herein are methods for rescuing terminally exhausted T cells and/or progenitor exhausted T cells and driving said T cells to an effector-like state further comprising monitoring the differentiation state of CD8+ T cells in the subject by flow cytometry analysis of T cell markers including GZMB, TOX, TCF1, and PD-1.

In one aspect, the methods of reducing, inhibiting, decreasing, treating, and/or preventing T cell exhaustion and/or rescuing terminally exhausted and/or progenitor exhausted T cells can include administration of an agent that inhibits, disrupts, reduces, and/or decreases signaling by the androgen receptor. Such inhibitors can include but are not limited to genetic manipulation (such as, for example, anti-AR antibody, CRISPR/Cas9-mediated knockout of the AR gene in the CD8+ T cells) and/or androgen deprivation therapy including, but not limited to the administration of small molecules, siRNA, shRNA, RNAi, anti-sense oligonucleotide, peptide, protein, or antibody that inhibits AR signaling). In one aspect, the androgen deprivation therapy comprises administration of Luteinizing Hormone-Releasing Hormone (LHRH) agonists comprising but not limited to leuprolide, goserelin, and triptorelin; the administration of androgen inhibitors comprising but not limited to bicalutamide, enzalutamide, apalutamide, flutamide, darolutamide, nilutamide, abiraterone, degarelix, relugolix, leuprolide, or goserelin; or administration of Androgen Synthesis Inhibitors comprising but not limited to abiraterone.

The disclosed methods can be used to rescue, inhibit, reduce, decrease, and/or prevent T cell exhaustion in any tumor microenvironment where uncontrolled proliferation occurs excluding prostate cancer. A representative but non-limiting list of cancers that the disclosed compositions can be used with is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers. Thus disclosed herein are methods of treating, decreasing, reducing, inhibiting, ameliorating, and/or preventing a cancer and/or metastasis wherein the cancer comprises T cell lymphoma; mycosis fungoides; Hodgkin's Disease; acute myeloid leukemia (AML); chronic myeloid leukemia (CML); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; skin cancer; hepatic cancer; melanoma; squamous cell carcinomas of the mouth, throat, larynx, and lung; cervical cancer; cervical carcinoma; breast cancer, triple negative breast cancer; genitourinary cancer; pulmonary cancer; esophageal carcinoma; head and neck carcinoma; large bowel cancer; hematopoietic cancers; testicular cancer; and colon and rectal cancer. In one aspect, the cancer is not prostate cancer.

Because the identification of exhausted phenotype T cells in the tumor environment would be an indicia that T cell exhaustion could be present and should be inhibited, the disclosed methods can further comprise detecting TOX+ and/or TCF1+ T cells. Therefore, also disclosed herein are methods of reducing, inhibiting, decreasing, treating, and/or preventing T cell exhaustion in the tumor microenvironment, further comprising assaying the T cells for being TOX+ and/or TCF1+; wherein the condition of being TOX+ and/or TCF1+ indicates that the T cell is exhausted. Detection of TOX+ and/or TCF1+ T cells can be achieved by any means known in the art, including, but not limited to immunofluorescence, flow cytometry, RNA sequencing, microarrays, enzyme linked immunoassay (ELISA), enzyme linked immunospot assay (ELIspot), reporter assays (such a s, for example, a luciferase reporter assay), and/or polymerase chain reaction (PCR).

It is understood and herein contemplated that the rescuing of a T cell from an exhausted state and driving said T cell to an effector state creates an enhancement of T cell effectors that is not limited to cancers, but applicable to any use where enhancing T cell differentiation to effector cells (and as a consequence increasing the numbers of effector T cells) is desired. Accordingly, disclosed herein are methods for promoting/enhancing differentiation of T cells (such as, for example, CD8+ T cells) to an effector state in a subject comprising administering to the subject an inhibitor of androgen receptor (AR) (including, but not limited to genetic manipulation (such as, for example, anti-AR antibody, CRISPR/Cas9-mediated knockout of the AR gene in the CD8+ T cells) or androgen deprivation therapy) directed to an AR on the T cells of the subject. In one aspect, the androgen deprivation therapy comprises administration of Luteinizing Hormone-Releasing Hormone (LHRH) agonists comprising but not limited to leuprolide, goserelin, and triptorelin; the administration of androgen inhibitors comprising but not limited to bicalutamide, enzalutamide, apalutamide, flutamide, darolutamide, nilutamide, abiraterone, degarelix, relugolix, leuprolide, or goserelin; or administration of Androgen Synthesis Inhibitors comprising but not limited to abiraterone.

### D. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular androgen receptor inhibitor is disclosed and discussed and a number of modifications that can be made to a number of molecules including the androgen receptor inhibitor are discussed, specifically contemplated is each and every combination and permutation of androgen receptor inhibitor and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### 1. Nucleic Acids

There are a variety of molecules disclosed herein that are nucleic acid based, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

### a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3-AMP (3-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### b) Sequences

There are a variety of sequences related to the protein molecules involved in the signaling pathways disclosed herein, for example androgen receptor signaling pathway, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those of skill in the art understand how to resolve sequence discrepancies and differences and to adjust the compositions and methods relating to a particular sequence to other related sequences. Primers and/or probes can be designed for any given sequence given the information disclosed herein and known in the art.

### c) Primers and Probes

Disclosed are compositions including primers and probes, which are capable of interacting with the disclosed nucleic acids, such as the androgen receptor as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

### d) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of any of the disclosed nucleic acids, such as tox, tcf7, AR, Havcr2, Isg15, Sry, 9-β-actin, hDNase NC2-chr13, or hTcf7 hAR_3. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

### 2. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with the androgen receptor (AR) such that AR signalling is inhibited. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, scFv, and the like, including hybrid fragments. Thus, fragments of the antibodies that retain the ability to bind their specific antigens are provided. For example, fragments of antibodies which maintain AR binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing anti AR antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 3. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Androgen Conspires with the CD8⁺ T Cell Exhaustion Program and Contributes to Sex Bias in Cancer

### a) Results

### (1) CD8⁺ T Cell Immunity Mediates Sex Differences in Multiple Pre-Clinical Cancer Models

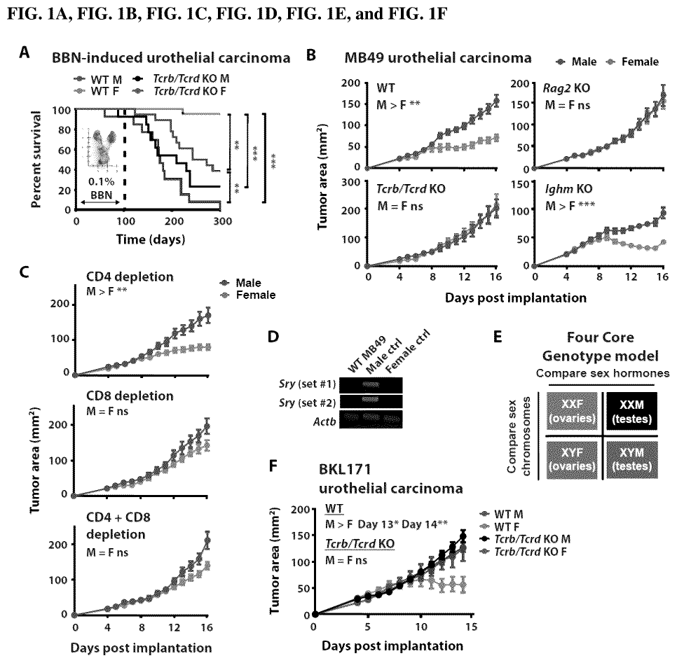

Upon ad libitum exposure to N-butyl-N-(4-hydroxybutyl) nitrosamine (BBN), a chemical carcinogen similar to those present in tobacco, mice reproducibly develop bladder cancer of similar histopathology and molecular aberrations as human bladder cancer. The BBN model recapitulates the male-biased development and mortality of bladder cancer even though its DNA mutation rates have been shown to be comparable between sexes, which indicates that males can have an increased cancer risk beyond carcinogen exposure and consequent transformation of the affected cells. Given the known immunogenicity of bladder cancer, we sought to address the contribution of adaptive immunity to sex bias. Consistent with earlier reports, wild type (WT) females survived longer after BBN exposure (FIG. 1A). However, BBN-induced carcinogenesis was similar in kinetics between male and female mice that are deficient of T cells (Tcrb/Tcrd^{-/-}) (FIG. 1A).

Male-biased tumor burden has been reported with the transplantable syngeneic bladder cancer cell line MB49. We evaluated the pertinent role of adaptive immunity by comparing MB49 growth in male and female WT mice, as well as mice with a deficiency of T cells (Tcrb/Tcrd^{-/-}), B cells (Ighm^{-/-}) or both (Rag2^{-/-}). We found MB49 grew more aggressively in male versus female WT mice (FIG. 1B). However, sex differences in MB49 growth were eliminated in Rag2 and Tcrb/Tcrd, but not Ighm, knockout (KO) mice, indicating that the sexual dimorphism is driven by endogenous anti-tumor T cell immunity (FIG. 1B). To evaluate the relative contributions from CD4⁺ and CD8⁺ T cells, we challenged WT mice with MB49 after CD4⁺ and/or CD8⁺ T cell antibody-mediated depletion. We found that tumor growth was comparable in male and female mice in the absence of CD8⁺, but not CD4⁺, T cells (FIG. 1C). While MB49 was generated through in vitro carcinogenesis of male mouse urothelial cells, enhanced immune responses in female mice are not due to H-Y minor antigens, as MB49 has lost the Y chromosome and therefore lacks Sry (FIG. 1D), a gene involved in the development of male gonadogenesis located on the Y chromosome. Further, we utilized the Four Core Genotype model, which includes wild type XX females and XY males, as well as sex-reversed XX males (due to transgenic autosome expression of Sry) and XY females (due to Sry knockout), to generate a BBN-induced bladder cancer cell line “BKL171” from an XX male (FIG. 1E). A clear male-biased tumor growth was also seen with BKL171 that was similarly dependent on T cell immunity, demonstrating that the sex-biased tumor growth is not the consequence of an alloresponse to Y chromosome-derived antigens by females (FIG. 1F).

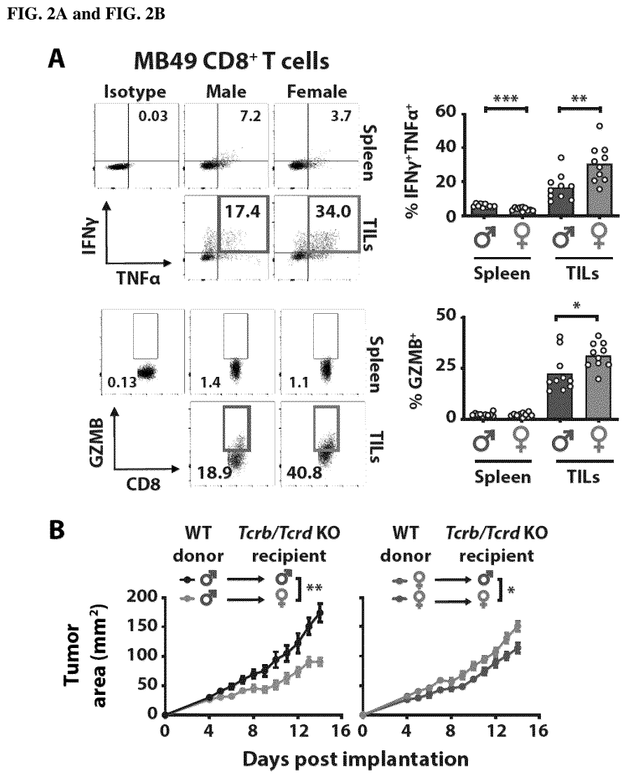

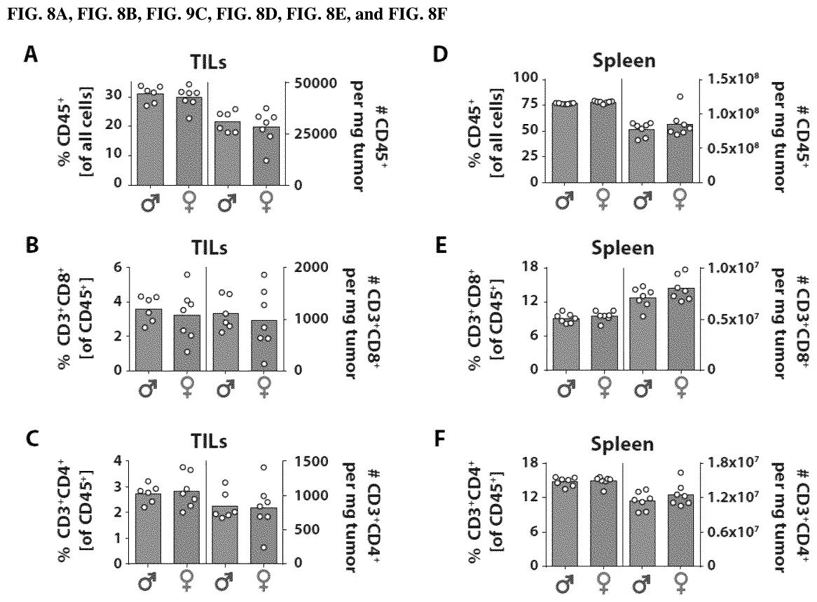

We hypothesized that fundamental sex differences exist in the effector function of CD8⁺ tumor-infiltrating lymphocytes (TILs). Since immune phenotypes often correlate with the magnitude of tumor burden, we focused our analysis on Days 9 to 11 post tumor implantation, which coincided with the time of bifurcation in tumor growth curves for males and females (FIG. 1B) and would identify a sex-specific driver—not passenger—phenotype. We observed that the number and frequency of tumor-infiltrating (FIGS. 8A, 8B, and 8C) and peripheral (FIGS. 8D, 8E, and 8F) CD45⁺, CD4⁺ and CD8⁺ T cells are comparable between sexes. However, compared to female mice, male mice had an approximately two-fold lower frequency of polyfunctional CD8⁺ T cells that could produce interferon gamma (IFNγ), tumor necrosis factor alpha (TNFα) and granzyme B (GZMB) in Day 9 tumors, but not in spleens (FIG. 2A). Further, to directly compare the male and female T cells in their anti-tumor immunity, we isolated tumor-reactive CD8⁺ T cells from the draining lymph nodes of WT mice and adoptively transferred them into MB49-bearing Tcrb/Tcrd KO recipient mice (FIG. 2B). We observed inferiority of CD8⁺ TILs in controlling tumor growth in male versus female mice, particularly with the use of male donor T cells. Collectively, our findings using multiple murine cancer models highlight a previously unappreciated role for sex differences in T cell immunity that mediates male-biased tumor burden.

### (2) Sex Differences in CD8⁺ T Cell Dynamics at Single-Cell Resolution in the Tumor Microenvironment

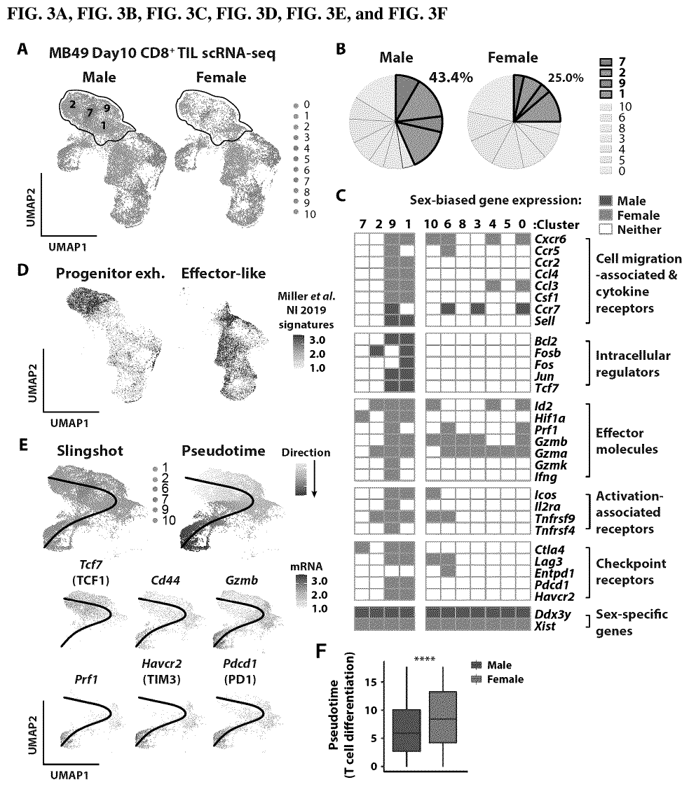

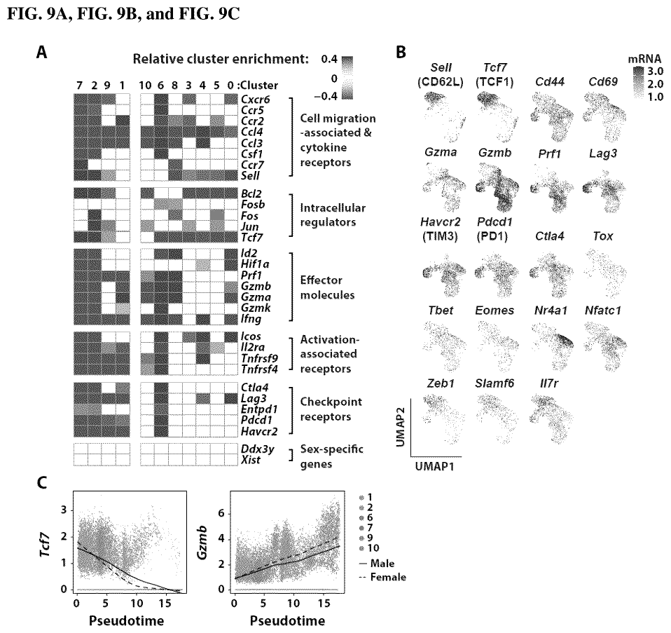

To better understand sex differences in CD8⁺ TILs, we performed single-cell RNA sequencing (scRNA-seq) on 26,698 CD8⁺ T cells that were FACS-sorted from Day 10 MB49 tumors (9,955 from female and 16,743 from male; n=3 mice per sex). The shared nearest neighbor modularity optimization-based clustering algorithm resolved 11 major clusters of cells at various stages of CD8⁺ T cell differentiation (FIG. 3A, FIGS. 9A and 9B, and table 1). Clusters 1, 2, 7 and 9 showed a male-biased frequency, comprising of 43.4% of all cells in males, in comparison to 25.0% in females (FIG. 3B). Consistent with the active anti-tumor immune response in this model, all but cell clusters 2 and 7 showed signs of activation via the T cell receptor (TCR), as marked by the expression of co-stimulatory receptors (Cd28, Icos, Il2ra), inhibitory surface receptors (Pdcd1, Havcr2, Lag3, Ctla4) and effector molecules (Ifng, Gzma, Gzmb). Clusters 2 and 7 were instead enriched for Tcf7, Sell, Bcl2, and ribosome products, which are indicative of stem-like properties. Clusters 3, 5 and 9 were distinguished by the expression of genes associated with active cell proliferation (Top2a, Mki67, Histones, Tubulins and MCM complex) and type I Interferon (IFN) signaling, respectively. Tox was not enriched in any clusters at this time point, consistent with a lack of their terminal differentiation in the early phase of tumor growth. Notably, clusters 1 and 9 showed a striking sex difference in their gene expression pattern (FIG. 3C). They showed a male-biased expression of Tcf7, Sell, Bcl2 and Jun transcripts. By comparison, in females, these same clusters were enriched for co-stimulatory receptors (Icos and Tnfrsf9), inhibitory surface receptors (Pdcd1, Havcr2, Lag3 and Ctla4), effector molecules (Gzma and Gzmb), transcription factors (Hifla and Id2). chemokines/cytokines (Ccl3, Ccl4 and Csf1) and migratory receptors (Ccr2 and Cxcr6).

Tcf7⁺ clusters 2 and 7 were enriched for a known signature of PE versus effector-like CD8⁺ T cells (FIG. 3D). They were not naïve as evidenced by high expression of Cd44 and Cd69 (FIG. 9B). Given that TCF1/Tcf7 plays a critical role in orchestrating a PE fate while antagonizing an effector program at the early stage of CD8⁺ T cell differentiation, we postulated that the male-biased Tcf7 gene expression in clusters 1 and 9 indicates an inflection at the stage male and female CD8⁺ T cells diverge to adopt a PE versus effector-like fate, respectively. To infer their ontogeny, we used the Slingshot algorithm to order single cells in clusters 1, 2, 6, 7, 9 and 10 in pseudotime (FIG. 3E). This analysis revealed distinct trajectories of CD8⁺ TIL differentiation by sexes, in which male CD8⁺ T cells were more likely to be found in earlier pseudotime with Tcf7 expression (FIG. 3F). Consistently, male CD8⁺ T cells had a tendency to retain Tcf7 expression and were less efficient in acquiring Gzmb expression over the pseudotime trajectory (FIG. 9C). These observations indicate that male and female CD8⁺ T cells are favored to adopt a PE versus effector fate, respectively.

TABLE 1
Seurat_list_markers. Gene markers for 11 clusters of CD8⁺ T cells from Day 10 MB49 tumors.

| | Female_p_val | Female_avg_logFC | Female_pct.1 | Femae_pct.2 | Female_p_val_adj | Male_p_val |
|---|---|---|---|---|---|---|
| mt-Co1 | 6.95E−142 | 1.176223849 | 0.995 | 1 | 1.08E−137 | 7.42E−235 |
| mt-Co2 | 7.39E−139 | 1.189672148 | 0.995 | 1 | 1.15E−134 | 1.51E−216 |
| mt-Atp6 | 1.07E−127 | 1.179119872 | 0.998 | 1 | 1.68E−123 | 3.11E−204 |
| mt-Co3 | 1.46E−130 | 1.200270449 | 0.998 | 1 | 2.27E−126 | 2.12E−198 |
| Rpl10 | 9.41E−138 | −0.92361087 | 0.722 | 0.968 | 1.47E−133 | 4.85E−193 |
| Rps2 | 2.07E−123 | −0.89140887 | 0.825 | 0.977 | 3.23E−119 | 6.79E−189 |
| Rpl5 | 6.19E−117 | −0.70865035 | 0.807 | 0.967 | 9.65E−113 | 2.89E−181 |
| mt-Nd4 | 9.68E−103 | 1.148999943 | 0.97 | 0.993 | 1.51E−98 | 4.28E−180 |
| Eef1a1 | 2.76E−109 | −0.7043233 | 0.949 | 0.991 | 4.30E−105 | 2.06E−179 |
| mt-Nd1 | 1.06E−98 | 1.125752221 | 0.954 | 0.99 | 1.66E−94 | 1.61E−177 |
| Gnb2l1 | 1.72E−108 | −0.66174395 | 0.818 | 0.97 | 2.69E−104 | 4.31E−177 |
| mt-Cytb | 5.00E−98 | 1.179292173 | 0.97 | 0.992 | 7.81E−94 | 2.08E−176 |
| Rps15a | 5.15E−110 | −0.75772737 | 0.892 | 0.986 | 8.03E−106 | 6.25E−176 |
| Rpl7 | 9.19E−116 | −0.73254759 | 0.777 | 0.971 | 1.43E−111 | 1.69E−169 |
| Btf3 | 2.11E−101 | −0.77421018 | 0.455 | 0.93 | 3.28E−97 | 4.37E−169 |
| Rpl32 | 2.43E−93 | −0.60964485 | 0.922 | 0.98 | 3.79E−89 | 7.29E−169 |
| Rplp0 | 5.17E−118 | −0.74534401 | 0.839 | 0.976 | 8.07E−114 | 9.87E−166 |
| Rps13 | 3.84E−103 | −0.65436549 | 0.908 | 0.977 | 5.99E−99 | 3.46E−164 |
| Tpt1 | 5.93E−108 | −0.72198189 | 0.989 | 0.996 | 9.25E−104 | 2.16E−163 |
| Rps27a | 3.65E−116 | −0.7243427 | 0.867 | 0.98 | 5.70E−112 | 3.03E−163 |
| Gm42418 | 3.32E−126 | 1.791356822 | 0.982 | 0.992 | 5.18E−122 | 5.26E−163 |
| Rps25 | 9.86E−113 | −0.79146906 | 0.653 | 0.945 | 1.54E−108 | 3.03E−162 |
| Rps18 | 1.16E−89 | −0.59639488 | 0.892 | 0.979 | 1.81E−85 | 1.24E−160 |
| Rpl3 | 1.13E−100 | −0.67243926 | 0.805 | 0.965 | 1.76E−96 | 1.96E−160 |
| Rps3a1 | 3.00E−114 | −0.66768532 | 0.878 | 0.98 | 4.68E−110 | 2.11E−160 |
| Rpl9 | 4.81E−114 | −0.7105892 | 0.821 | 0.976 | 7.51E−110 | 1.05E−159 |
| Rpl12 | 3.94E−107 | −0.74724264 | 0.839 | 0.967 | 6.14E−103 | 1.09E−159 |
| Rpl23a | 6.86E−119 | −0.86187871 | 0.547 | 0.94 | 1.07E−114 | 1.27E−158 |
| Rps10 | 3.75E−112 | −0.68225872 | 0.885 | 0.975 | 5.86E−108 | 8.04E−158 |
| Rpl18 | 4.96E−105 | −0.64785555 | 0.83 | 0.981 | 7.74E−101 | 4.97E−157 |
| Rps7 | 5.66E−122 | −0.76981547 | 0.89 | 0.974 | 8.83E−118 | 7.52E−157 |
| Rpl39 | 6.11E−126 | −0.87573113 | 0.802 | 0.97 | 9.53E−122 | 1.18E−154 |
| Rpl30 | 1.59E−108 | −0.66763704 | 0.86 | 0.979 | 2.48E−104 | 2.38E−153 |
| mt-Nd2 | 2.29E−95 | 1.130166538 | 0.952 | 0.991 | 3.57E−91 | 5.10E−153 |
| Rpl23 | 2.35E−103 | −0.61609959 | 0.926 | 0.99 | 3.67E−99 | 8.51E−152 |
| Rps23 | 1.52E−88 | −0.60346443 | 0.876 | 0.977 | 2.38E−84 | 8.52E−152 |
| Rps6 | 4.81E−87 | −0.56377754 | 0.837 | 0.968 | 7.50E−83 | 1.33E−148 |
| Rpl15 | 5.68E−119 | −0.90460057 | 0.464 | 0.93 | 8.86E−115 | 1.55E−148 |
| Malat1 | 3.49E−89 | 0.982574382 | 0.982 | 0.999 | 5.45E−85 | 3.04E−148 |
| Rps3 | 3.73E−109 | −0.65015567 | 0.876 | 0.975 | 5.83E−105 | 2.16E−147 |
| Rps16 | 4.41E−95 | −0.63244282 | 0.892 | 0.982 | 6.87E−91 | 5.14E−145 |
| Rps24 | 3.32E−104 | −0.72230356 | 0.906 | 0.983 | 5.18E−100 | 8.05E−143 |
| Eef1b2 | 7.53E−118 | −0.87673079 | 0.54 | 0.949 | 1.17E−113 | 1.23E−142 |
| Eif1 | 7.21E−92 | −0.65640093 | 0.699 | 0.968 | 1.13E−87 | 4.72E−142 |
| Rps20 | 4.09E−91 | −0.64021398 | 0.869 | 0.982 | 6.38E−87 | 9.23E−142 |
| Ubb | 4.61E−97 | −0.72000964 | 0.726 | 0.954 | 7.20E−93 | 1.10E−141 |
| Rpl27a | 2.14E−84 | −0.54396355 | 0.917 | 0.985 | 3.34E−80 | 5.76E−141 |
| Rpl34 | 6.89E−96 | −0.67865988 | 0.805 | 0.969 | 1.08E−91 | 2.77E−140 |
| Rps4x | 4.33E−81 | −0.54692954 | 0.834 | 0.967 | 6.75E−77 | 4.03E−139 |
| Ppia | 3.50E−127 | −0.90959052 | 0.628 | 0.966 | 5.47E−123 | 1.91E−138 |
| Rplp2 | 3.63E−94 | −0.57087614 | 0.887 | 0.977 | 5.66E−90 | 2.38E−137 |
| Naca | 2.95E−93 | −0.67011313 | 0.572 | 0.944 | 4.60E−89 | 1.86E−134 |
| mt-Nd5 | 1.84E−79 | 1.101780801 | 0.857 | 0.936 | 2.87E−75 | 1.34E−133 |
| Rpl36a | 2.70E−91 | −0.72690602 | 0.561 | 0.941 | 4.22E−87 | 2.23E−133 |
| Rps9 | 2.70E−82 | −0.54308576 | 0.851 | 0.972 | 4.21E−78 | 6.79E−133 |
| Rps14 | 3.29E−84 | −0.5920885 | 0.878 | 0.972 | 5.13E−80 | 9.54E−133 |
| Rbm3 | 8.12E−95 | −0.75684404 | 0.501 | 0.935 | 1.27E−90 | 2.26E−131 |
| Rps11 | 1.03E−69 | −0.4612956 | 0.883 | 0.971 | 1.61E−65 | 3.90E−131 |
| Rpl11 | 1.43E−83 | −0.50489217 | 0.869 | 0.979 | 2.24E−79 | 5.21E−131 |
| Rpl29 | 9.57E−86 | −0.59971339 | 0.662 | 0.947 | 1.49E−81 | 1.15E−129 |
| Rpl18a | 5.20E−72 | −0.49190826 | 0.899 | 0.981 | 8.12E−68 | 1.03E−128 |
| Tmsb4x | 1.28E−93 | −0.76056172 | 0.991 | 0.999 | 2.00E−89 | 1.86E−128 |
| Pfn1 | 7.30E−87 | −0.68912361 | 0.72 | 0.944 | 1.14E−82 | 2.14E−128 |
| Rps12 | 6.10E−70 | −0.53093184 | 0.878 | 0.971 | 9.51E−66 | 5.15E−128 |
| Rpl35 | 8.63E−78 | −0.6306061 | 0.637 | 0.943 | 1.35E−73 | 5.33E−128 |
| Cox6a1 | 7.89E−81 | −0.47926885 | 0.166 | 0.792 | 1.23E−76 | 3.80E−127 |
| Cox5a | 3.22E−93 | −0.70689861 | 0.244 | 0.871 | 5.02E−89 | 4.71E−127 |
| Erh | 1.45E−88 | −0.64597828 | 0.161 | 0.791 | 2.26E−84 | 6.10E−127 |
| Rps5 | 6.57E−87 | −0.58351074 | 0.848 | 0.969 | 1.02E−82 | 7.24E−126 |
| Rpl37 | 7.41E−73 | −0.50561982 | 0.823 | 0.966 | 1.16E−68 | 8.13E−126 |
| Rps26 | 2.30E−74 | −0.56258743 | 0.77 | 0.961 | 3.59E−70 | 2.50E−125 |
| Snrpf | 8.80E−59 | −0.42110665 | 0.251 | 0.822 | 1.37E−54 | 1.22E−124 |
| Rps29 | 2.10E−70 | −0.55165655 | 0.83 | 0.969 | 3.27E−66 | 2.40E−124 |
| Rps17 | 1.80E−85 | −0.75003833 | 0.49 | 0.927 | 2.81E−81 | 5.73E−124 |
| Atp5g1 | 9.16E−79 | −0.5374329 | 0.159 | 0.767 | 1.43E−74 | 2.02E−123 |
| Gabarap | 1.01E−55 | −0.36994764 | 0.271 | 0.857 | 1.58E−51 | 3.17E−123 |
| Rpl13 | 3.82E−75 | −0.48861419 | 0.947 | 0.99 | 5.96E−71 | 2.19E−122 |
| Ndufa4 | 1.58E−85 | −0.6236469 | 0.209 | 0.841 | 2.47E−81 | 3.30E−122 |
| Rpl19 | 1.19E−63 | −0.46096339 | 0.88 | 0.978 | 1.86E−59 | 3.02E−121 |
| Sumo2 | 2.24E−76 | −0.56034208 | 0.278 | 0.892 | 3.49E−72 | 5.86E−121 |
| Eif3i | 7.00E−69 | −0.42564503 | 0.202 | 0.805 | 1.09E−64 | 5.79E−120 |
| Rpl17 | 1.40E−49 | −0.40287227 | 0.917 | 0.977 | 2.19E−45 | 1.48E−119 |
| Prdx1 | 3.35E−83 | −0.5575613 | 0.166 | 0.788 | 5.23E−79 | 1.99E−119 |
| Tbca | 1.29E−85 | −0.43083605 | 0.117 | 0.739 | 2.01E−81 | 3.66E−119 |
| Psma2 | 1.44E−83 | −0.56615781 | 0.177 | 0.813 | 2.25E−79 | 1.00E−117 |
| H3f3a | 4.16E−90 | −0.70384088 | 0.464 | 0.935 | 6.48E−86 | 4.98E−117 |
| Slc25a4 | 2.30E−64 | −0.35559932 | 0.193 | 0.778 | 3.59E−60 | 7.59E−117 |
| Sf3b6 | 1.18E−88 | −0.42432852 | 0.113 | 0.738 | 1.84E−84 | 3.80E−116 |
| Psme2 | 1.68E−58 | −0.33673726 | 0.228 | 0.806 | 2.62E−54 | 5.37E−116 |
| Atp5c1 | 1.94E−67 | −0.50395954 | 0.239 | 0.843 | 3.03E−63 | 6.91E−116 |
| Rplp1 | 3.49E−76 | −0.54985951 | 0.903 | 0.976 | 5.44E−72 | 7.55E−116 |
| Cox7b | 4.58E−89 | −0.58966173 | 0.175 | 0.815 | 7.14E−85 | 7.75E−115 |
| Rpl14 | 3.20E−70 | −0.53140371 | 0.706 | 0.953 | 4.99E−66 | 9.99E−115 |
| Npm1 | 9.19E−88 | −0.82086007 | 0.499 | 0.937 | 1.43E−83 | 1.29E−114 |
| Rps21 | 2.55E−72 | −0.56190257 | 0.795 | 0.978 | 3.97E−68 | 1.44E−114 |
| mt-Nd3 | 7.70E−67 | 1.068872294 | 0.844 | 0.928 | 1.20E−62 | 1.79E−114 |
| Tagln2 | 1.23E−85 | −0.70867022 | 0.191 | 0.803 | 1.92E−81 | 1.85E−114 |
| Rpl22 | 7.36E−78 | −0.56039792 | 0.68 | 0.946 | 1.15E−73 | 1.97E−114 |
| Slc25a5 | 1.46E−113 | −0.90451901 | 0.205 | 0.874 | 2.28E−109 | 3.98E−87 |
| Edf1 | 9.14E−46 | −0.28162426 | 0.269 | 0.81 | 1.43E−41 | 1.76E−113 |
| Ran | 7.66E−85 | −0.69042703 | 0.23 | 0.839 | 1.19E−80 | 1.77E−113 |
| Eif3e | 4.04E−64 | −0.50165807 | 0.301 | 0.88 | 6.31E−60 | 2.60E−113 |
| Anp32a | 4.02E−93 | −0.53341379 | 0.115 | 0.751 | 6.26E−89 | 5.20E−113 |
| Eif5a | 1.79E−70 | −0.70106742 | 0.34 | 0.88 | 2.80E−66 | 1.26E−112 |
| Ndufa7 | 9.91E−71 | −0.31575186 | 0.163 | 0.766 | 1.55E−66 | 1.29E−112 |
| Rpl27 | 2.73E−101 | −0.72979072 | 0.609 | 0.941 | 4.26E−97 | 3.98E−112 |
| Cox5b | 5.01E−66 | −0.48318441 | 0.241 | 0.835 | 7.82E−62 | 2.41E−111 |
| Txn1 | 1.68E−92 | −0.78818583 | 0.26 | 0.87 | 2.62E−88 | 4.14E−111 |
| Uqcrb | 5.93E−83 | −0.4870726 | 0.129 | 0.745 | 9.26E−79 | 6.60E−111 |
| Snrpd1 | 6.09E−88 | −0.53291007 | 0.085 | 0.678 | 9.50E−84 | 7.55E−111 |
| Rpl22l1 | 6.98E−81 | −0.68851796 | 0.428 | 0.916 | 1.09E−76 | 9.50E−111 |
| Cox8a | 1.64E−98 | −0.75006868 | 0.428 | 0.93 | 2.56E−94 | 1.17E−110 |
| Rpl8 | 1.96E−47 | −0.35024564 | 0.89 | 0.978 | 3.06E−43 | 1.32E−110 |
| Ndufa6 | 2.25E−72 | −0.39964211 | 0.193 | 0.811 | 3.50E−68 | 1.69E−110 |
| Cox7a2 | 1.99E−65 | −0.47380429 | 0.255 | 0.852 | 3.10E−61 | 1.97E−110 |
| Arhgdib | 5.54E−70 | −0.63550815 | 0.375 | 0.893 | 8.65E−66 | 4.88E−110 |
| S100a11 | 1.59E−65 | −0.61869308 | 0.313 | 0.851 | 2.48E−61 | 9.41E−110 |
| Ost4 | 1.55E−88 | −0.50481859 | 0.12 | 0.743 | 2.43E−84 | 1.19E−109 |
| Usmg5 | 2.79E−85 | −0.4108528 | 0.092 | 0.693 | 4.36E−81 | 1.45E−109 |
| Psmb3 | 2.43E−79 | −0.555131 | 0.191 | 0.818 | 3.78E−75 | 2.19E−109 |
| Snrpg | 2.37E−89 | −0.62736833 | 0.189 | 0.84 | 3.70E−85 | 3.99E−109 |
| Ndufb8 | 7.70E−69 | −0.35572727 | 0.159 | 0.736 | 1.20E−64 | 8.94E−109 |
| Myl12a | 3.99E−88 | −0.74068712 | 0.274 | 0.879 | 6.22E−84 | 1.17E−108 |
| Ndufb9 | 1.57E−74 | −0.45724478 | 0.166 | 0.776 | 2.46E−70 | 2.32E−108 |
| Magoh | 9.36E−89 | −0.52001984 | 0.069 | 0.658 | 1.46E−84 | 2.49E−108 |
| 2700060E02Rik | 1.86E−75 | −0.38226154 | 0.166 | 0.783 | 2.90E−71 | 8.45E−108 |
| Pomp | 7.34E−59 | −0.38221724 | 0.251 | 0.831 | 1.14E−54 | 2.15E−107 |
| Atp5l | 5.28E−78 | −0.60977858 | 0.308 | 0.894 | 8.24E−74 | 3.10E−107 |
| Psmb2 | 1.60E−85 | −0.51737667 | 0.101 | 0.698 | 2.50E−81 | 7.57E−107 |
| Ndufb11 | 5.71E−80 | −0.53511587 | 0.195 | 0.831 | 8.91E−76 | 7.65E−107 |
| Cfl1 | 3.32E−81 | −0.64136856 | 0.533 | 0.925 | 5.19E−77 | 8.38E−107 |
| Tomm22 | 1.21E−86 | −0.42063101 | 0.136 | 0.782 | 1.88E−82 | 1.53E−106 |
| Oaz1 | 2.15E−68 | −0.52615351 | 0.563 | 0.938 | 3.35E−64 | 2.00E−106 |
| Myl12b | 1.59E−67 | −0.50054096 | 0.271 | 0.866 | 2.48E−63 | 2.46E−106 |
| Atp50 | 1.41E−58 | −0.29133595 | 0.2 | 0.772 | 2.20E−54 | 3.48E−106 |
| Snrpd2 | 5.30E−59 | −0.32034332 | 0.2 | 0.771 | 8.27E−55 | 6.39E−106 |
| Cct8 | 4.11E−79 | −0.43563886 | 0.122 | 0.711 | 6.42E−75 | 7.67E−106 |
| Rpl10a | 1.24E−48 | −0.37432936 | 0.834 | 0.963 | 1.94E−44 | 1.16E−105 |
| Brk1 | 8.62E−78 | −0.3825518 | 0.145 | 0.761 | 1.34E−73 | 1.65E−105 |
| Snx3 | 1.10E−71 | −0.31816235 | 0.131 | 0.709 | 1.72E−67 | 2.70E−105 |
| Sin3b | 4.32E−72 | −0.28128354 | 0.126 | 0.703 | 6.75E−68 | 5.05E−105 |
| Tspo | 4.17E−67 | −0.4913616 | 0.274 | 0.885 | 6.51E−63 | 8.03E−105 |
| Nhp2l1 | 3.04E−83 | −0.46072763 | 0.108 | 0.708 | 4.74E−79 | 8.26E−105 |
| Lamtor5 | 2.46E−79 | −0.39927748 | 0.083 | 0.651 | 3.83E−75 | 1.48E−104 |
| Sri | 1.47E−74 | −0.44917511 | 0.189 | 0.802 | 2.30E−70 | 6.98E−104 |
| Phb2 | 4.66E−62 | −0.27786312 | 0.161 | 0.72 | 7.27E−58 | 8.37E−104 |
| Nme1 | 3.80E−70 | −0.41023687 | 0.145 | 0.709 | 5.93E−66 | 9.32E−104 |
| Cycs | 5.82E−81 | −0.55888328 | 0.156 | 0.76 | 9.08E−77 | 1.32E−103 |
| Atp5j | 4.63E−62 | −0.45798119 | 0.255 | 0.838 | 7.22E−58 | 1.56E−103 |
| Gabarapl2 | 5.08E−83 | −0.40309719 | 0.133 | 0.757 | 7.92E−79 | 1.59E−103 |
| Psma7 | 7.54E−68 | −0.39632644 | 0.218 | 0.82 | 1.18E−63 | 2.03E−103 |
| Taldo1 | 2.33E−84 | −0.3830258 | 0.097 | 0.692 | 3.64E−80 | 2.07E−103 |
| Snrpe | 4.64E−98 | −0.75699416 | 0.232 | 0.883 | 7.24E−94 | 2.87E−103 |
| Ndufb5 | 7.53E−85 | −0.44062821 | 0.147 | 0.783 | 1.18E−80 | 3.16E−103 |
| Psmb5 | 6.88E−75 | −0.41351963 | 0.133 | 0.723 | 1.07E−70 | 6.17E−103 |
| Ube2l3 | 1.90E−79 | −0.44762213 | 0.149 | 0.769 | 2.97E−75 | 8.45E−103 |
| Psma5 | 5.14E−74 | −0.30914318 | 0.094 | 0.652 | 8.02E−70 | 8.85E−103 |
| Rps28 | 2.59E−76 | −0.597253 | 0.572 | 0.934 | 4.05E−72 | 9.20E−103 |
| Mpc1 | 1.45E−79 | −0.43245743 | 0.044 | 0.578 | 2.26E−75 | 2.61E−102 |
| Spcs1 | 3.28E−92 | −0.50705916 | 0.113 | 0.749 | 5.12E−88 | 5.53E−102 |
| Atp5f1 | 1.16E−80 | −0.64370936 | 0.239 | 0.856 | 1.82E−76 | 6.57E−102 |
| Tceb1 | 2.83E−73 | −0.37494618 | 0.133 | 0.722 | 4.41E−69 | 8.65E−102 |
| Psmb1 | 1.89E−63 | −0.51168813 | 0.301 | 0.892 | 2.94E−59 | 1.29E−101 |
| Rps27l | 2.15E−77 | −0.45906101 | 0.129 | 0.716 | 3.35E−73 | 1.65E−101 |
| Atp6v1f | 7.94E−72 | −0.40544672 | 0.202 | 0.822 | 1.24E−67 | 2.34E−101 |
| Mrpl20 | 3.20E−91 | −0.45421464 | 0.071 | 0.677 | 5.00E−87 | 3.40E−101 |
| Rbx1 | 1.17E−72 | −0.45538419 | 0.179 | 0.797 | 1.82E−68 | 5.29E−101 |
| Lsm5 | 1.61E−77 | −0.45517773 | 0.074 | 0.622 | 2.52E−73 | 5.43E−101 |
| Ssr4 | 1.04E−87 | −0.5811823 | 0.184 | 0.84 | 1.62E−83 | 7.48E−101 |
| Mrps14 | 1.24E−79 | −0.42762371 | 0.085 | 0.654 | 1.94E−75 | 1.01E−100 |
| Sec61b | 1.26E−72 | −0.59507133 | 0.294 | 0.884 | 1.96E−68 | 1.36E−100 |
| Cox6c | 8.18E−81 | −0.65990441 | 0.287 | 0.89 | 1.28E−76 | 1.67E−100 |
| Mettl23 | 4.84E−69 | −0.28306928 | 0.163 | 0.761 | 7.56E−65 | 2.43E−100 |
| Ywhaq | 1.94E−70 | −0.30327184 | 0.117 | 0.68 | 3.03E−66 | 2.52E−100 |
| Rpl24 | 6.31E−47 | −0.37610151 | 0.832 | 0.969 | 9.84E−43 | 3.31E−100 |
| Chchd2 | 3.62E−61 | −0.53920917 | 0.384 | 0.912 | 5.64E−57 | 3.88E−100 |
| Rps8 | 9.18E−45 | −0.359549 | 0.917 | 0.981 | 1.43E−40 | 3.92E−100 |
| Gng5 | 1.12E−76 | −0.5320735 | 0.225 | 0.869 | 1.75E−72 | 4.40E−100 |
| Snrpb | 6.64E−65 | −0.41136035 | 0.209 | 0.8 | 1.04E−60 | 4.79E−100 |
| Nedd8 | 3.91E−68 | −0.4149811 | 0.214 | 0.813 | 6.10E−64 | 5.82E−100 |
| Pdcd6 | 1.24E−71 | −0.28712202 | 0.108 | 0.671 | 1.94E−67 | 6.69E−100 |
| Esd | 5.46E−82 | −0.42162316 | 0.129 | 0.739 | 8.52E−78 | 2.54E−99 |
| Arpc3 | 7.02E−57 | −0.51439345 | 0.368 | 0.904 | 1.10E−52 | 2.65E−99 |
| Gm10073 | 2.07E−91 | −0.51548028 | 0.078 | 0.682 | 3.22E−87 | 5.31E−99 |
| Aprt | 2.42E−79 | −0.38215205 | 0.113 | 0.698 | 3.77E−75 | 5.55E−99 |
| Rpl21 | 3.82E−46 | −0.35475377 | 0.851 | 0.966 | 5.95E−42 | 6.48E−99 |
| Minos1 | 2.13E−64 | −0.31480985 | 0.175 | 0.764 | 3.33E−60 | 8.49E−99 |
| Srp14 | 2.88E−74 | −0.43251967 | 0.189 | 0.807 | 4.50E−70 | 1.89E−98 |
| Nop10 | 3.86E−80 | −0.45318666 | 0.074 | 0.634 | 6.02E−76 | 2.08E−98 |
| 2410015M20Rik | 3.87E−62 | −0.27850044 | 0.179 | 0.758 | 6.04E−58 | 2.91E−98 |
| Npm3 | 3.81E−68 | −0.35659214 | 0.083 | 0.597 | 5.94E−64 | 3.11E−98 |
| Ndufab1 | 2.65E−64 | −0.31857939 | 0.101 | 0.622 | 4.13E−60 | 4.93E−98 |
| Pebp1 | 6.02E−71 | −0.33846839 | 0.14 | 0.719 | 9.39E−67 | 2.13E−97 |
| Rpl23a-ps3 | 1.92E−74 | −0.39038602 | 0.051 | 0.569 | 3.00E−70 | 2.22E−97 |
| Cdk2ap2 | 6.10E−69 | −0.28547856 | 0.126 | 0.689 | 9.51E−65 | 2.56E−97 |
| Cct2 | 5.30E−68 | −0.44950275 | 0.207 | 0.81 | 8.27E−64 | 4.65E−97 |
| Mif | 3.11E−64 | −0.64723485 | 0.372 | 0.873 | 4.85E−60 | 4.91E−97 |
| Manf | 8.51E−79 | −0.49831539 | 0.126 | 0.72 | 1.33E−74 | 7.98E−97 |
| 15-Sep | 1.96E−95 | −0.55155774 | 0.122 | 0.777 | 3.06E−91 | 1.40E−96 |
| Snrpd3 | 2.81E−69 | −0.34074302 | 0.087 | 0.613 | 4.38E−65 | 1.40E−96 |
| Atp6v0e | 1.00E−68 | −0.44228661 | 0.216 | 0.83 | 1.56E−64 | 1.42E−96 |
| Hprt | 1.99E−81 | −0.43028765 | 0.101 | 0.689 | 3.11E−77 | 1.50E−96 |
| 2010107E04Rik | 1.71E−55 | −0.34605132 | 0.228 | 0.809 | 2.67E−51 | 1.51E−96 |
| Bax | 6.30E−73 | −0.33136151 | 0.136 | 0.722 | 9.83E−69 | 1.88E−96 |
| Tmem258 | 8.08E−75 | −0.35986285 | 0.14 | 0.737 | 1.26E−70 | 2.55E−96 |
| Psmb6 | 9.02E−62 | −0.25939128 | 0.156 | 0.712 | 1.41E−57 | 6.03E−96 |
| Eif3m | 3.61E−76 | −0.47503558 | 0.214 | 0.841 | 5.64E−72 | 8.44E−96 |
| Srgn | 1.27E−92 | −0.83774997 | 0.563 | 0.982 | 1.98E−88 | 1.15E−95 |
| Banf1 | 7.88E−70 | −0.36810574 | 0.078 | 0.597 | 1.23E−65 | 2.00E−95 |
| Psmb9 | 3.91E−58 | −0.2785673 | 0.209 | 0.769 | 6.10E−54 | 3.33E−95 |
| Prdx5 | 2.00E−69 | −0.3696678 | 0.168 | 0.749 | 3.13E−65 | 3.82E−95 |
| Sap18 | 4.54E−86 | −0.37789359 | 0.094 | 0.697 | 7.08E−82 | 5.40E−95 |
| Ube2n | 1.39E−74 | −0.37407625 | 0.117 | 0.695 | 2.16E−70 | 7.59E−95 |
| Lsm4 | 2.86E−69 | −0.37803046 | 0.156 | 0.732 | 4.47E−65 | 1.34E−94 |
| Ndufa8 | 6.28E−88 | −0.45213796 | 0.078 | 0.673 | 9.79E−84 | 1.69E−94 |
| Mdh1 | 1.01E−59 | −0.28411787 | 0.152 | 0.689 | 1.58E−55 | 1.92E−94 |
| Swi5 | 3.51E−78 | −0.41589527 | 0.099 | 0.675 | 5.47E−74 | 1.97E−94 |
| Uqcrfs1 | 1.30E−73 | −0.35370197 | 0.143 | 0.735 | 2.02E−69 | 1.97E−94 |
| Vdac2 | 1.67E−68 | −0.31002338 | 0.166 | 0.756 | 2.61E−64 | 2.71E−94 |
| Mrpl33 | 3.63E−62 | −0.38429657 | 0.218 | 0.8 | 5.67E−58 | 3.45E−94 |
| Psmd8 | 9.35E−62 | −0.28834094 | 0.179 | 0.752 | 1.46E−57 | 4.06E−94 |
| Ptma | 1.49E−64 | −0.66851843 | 0.779 | 0.966 | 2.32E−60 | 6.35E−94 |
| Mrfap1 | 2.43E−72 | −0.32305836 | 0.136 | 0.719 | 3.80E−68 | 7.52E−94 |
| Fth1 | 1.48E−70 | −0.54002955 | 0.526 | 0.959 | 2.31E−66 | 9.21E−94 |
| S100a10 | 1.41E−66 | −0.67355285 | 0.474 | 0.914 | 2.20E−62 | 9.36E−94 |
| Eif3k | 6.42E−52 | −0.40630362 | 0.317 | 0.878 | 1.00E−47 | 1.61E−93 |
| Psma1 | 5.50E−69 | −0.33312746 | 0.147 | 0.726 | 8.58E−65 | 2.11E−93 |
| Txnl1 | 9.50E−84 | −0.40107584 | 0.08 | 0.66 | 1.48E−79 | 2.18E−93 |
| Tomm7 | 3.79E−74 | −0.46516711 | 0.2 | 0.816 | 5.92E−70 | 2.83E−93 |
| Rac1 | 2.07E−71 | −0.47499104 | 0.228 | 0.844 | 3.23E−67 | 2.98E−93 |
| Fkbp3 | 2.62E−67 | −0.32814332 | 0.106 | 0.643 | 4.09E−63 | 3.05E−93 |
| Erp44 | 1.66E−69 | −0.34204677 | 0.12 | 0.671 | 2.60E−65 | 5.51E−93 |
| Cacybp | 9.15E−76 | −0.40824331 | 0.085 | 0.636 | 1.43E−71 | 6.62E−93 |
| Pdcd5 | 1.82E−73 | −0.26251079 | 0.113 | 0.685 | 2.83E−69 | 7.54E−93 |
| Dnajc15 | 2.19E−67 | −0.48136932 | 0.246 | 0.839 | 3.41E−63 | 1.20E−92 |
| Uqcrc2 | 7.53E−84 | −0.37551914 | 0.085 | 0.672 | 1.18E−79 | 1.63E−92 |
| Smdt1 | 2.35E−59 | −0.30828616 | 0.177 | 0.734 | 3.67E−55 | 2.08E−92 |
| Rpl6 | 1.11E−41 | −0.33556332 | 0.8 | 0.959 | 1.73E−37 | 2.09E−92 |
| Mdh2 | 4.32E−82 | −0.50029556 | 0.126 | 0.73 | 6.73E−78 | 2.20E−92 |
| Hint1 | 1.20E−77 | −0.62458633 | 0.274 | 0.869 | 1.87E−73 | 2.22E−92 |
| Cap1 | 1.21E−57 | −0.27009539 | 0.193 | 0.763 | 1.88E−53 | 6.02E−92 |
| Sh2d1a | 1.06E−69 | −0.368752 | 0.12 | 0.671 | 1.66E−65 | 8.32E−92 |
| 1110008F13Rik | 4.13E−65 | −0.28672346 | 0.12 | 0.663 | 6.45E−61 | 1.72E−91 |
| Rpl4 | 1.77E−44 | −0.34720913 | 0.772 | 0.951 | 2.76E−40 | 1.89E−91 |
| Tmem14c | 1.65E−83 | −0.47509611 | 0.11 | 0.711 | 2.58E−79 | 3.62E−91 |
| Psenen | 3.40E−76 | −0.38082131 | 0.17 | 0.798 | 5.30E−72 | 3.76E−91 |
| Ndufv2 | 7.70E−72 | −0.31793694 | 0.085 | 0.625 | 1.20E−67 | 3.79E−91 |
| Rps19 | 1.68E−52 | −0.41680444 | 0.86 | 0.969 | 2.63E−48 | 4.00E−91 |
| Dap | 9.99E−62 | −0.31159137 | 0.131 | 0.663 | 1.56E−57 | 4.66E−91 |
| Eif6 | 2.30E−80 | −0.40019468 | 0.064 | 0.621 | 3.59E−76 | 5.33E−91 |
| Bzw1 | 2.20E−58 | −0.26258206 | 0.195 | 0.765 | 3.43E−54 | 5.33E−91 |
| Tomm5 | 5.08E−71 | −0.34834039 | 0.11 | 0.666 | 7.92E−67 | 8.26E−91 |
| Polr1d | 3.70E−67 | −0.51658151 | 0.267 | 0.864 | 5.77E−63 | 1.54E−90 |
| Eef1d | 1.59E−46 | −0.34555594 | 0.322 | 0.874 | 2.47E−42 | 1.60E−90 |
| Rpl35a | 1.67E−40 | −0.32891946 | 0.867 | 0.967 | 2.61E−36 | 2.20E−90 |
| Rpl7a | 2.20E−34 | −0.31539736 | 0.775 | 0.951 | 3.43E−30 | 3.14E−90 |
| Pgam1 | 6.28E−72 | −0.52561886 | 0.211 | 0.791 | 9.79E−68 | 3.82E−90 |
| Krtcap2 | 4.36E−73 | −0.56838886 | 0.244 | 0.865 | 6.80E−69 | 3.95E−90 |
| Rpl28 | 7.79E−46 | −0.39456681 | 0.855 | 0.969 | 1.22E−41 | 7.49E−90 |
| ler3ip1 | 1.08E−74 | −0.29200952 | 0.108 | 0.678 | 1.69E−70 | 7.70E−90 |
| Arf1 | 9.47E−90 | −0.56854125 | 0.182 | 0.843 | 1.48E−85 | 2.06E−83 |
| Shfm1 | 1.54E−51 | −0.42529177 | 0.315 | 0.866 | 2.40E−47 | 2.18E−89 |
| Eif1ax | 3.71E−77 | −0.4233999 | 0.09 | 0.654 | 5.79E−73 | 4.06E−89 |
| Atp5g3 | 3.14E−73 | −0.4443834 | 0.189 | 0.792 | 4.90E−69 | 4.27E−89 |
| Vapa | 2.10E−79 | −0.30721436 | 0.071 | 0.631 | 3.28E−75 | 4.97E−89 |
| Raly | 1.58E−63 | −0.32277901 | 0.163 | 0.738 | 2.47E−59 | 5.28E−89 |
| Emp3 | 2.99E−71 | −0.52468147 | 0.195 | 0.788 | 4.67E−67 | 9.75E−89 |
| Rpl38 | 1.46E−44 | −0.37712208 | 0.86 | 0.966 | 2.28E−40 | 1.06E−88 |
| Selk | 1.25E−62 | −0.34530853 | 0.246 | 0.846 | 1.95E−58 | 1.46E−88 |
| Ube2s | 9.75E−54 | −0.38148816 | 0.209 | 0.74 | 1.52E−49 | 1.49E−88 |
| Eif2s2 | 6.65E−56 | −0.39148982 | 0.251 | 0.81 | 1.04E−51 | 1.58E−88 |
| Calm1 | 1.04E−66 | −0.58386604 | 0.485 | 0.919 | 1.62E−62 | 1.71E−88 |
| Vps29 | 1.67E−66 | −0.25334826 | 0.101 | 0.633 | 2.61E−62 | 2.12E−88 |
| Tomm20 | 5.69E−69 | −0.54997477 | 0.32 | 0.907 | 8.88E−65 | 2.63E−88 |
| Cwc15 | 2.26E−63 | −0.27435773 | 0.156 | 0.717 | 3.52E−59 | 3.26E−88 |
| Fau | 1.06E−42 | −0.33512957 | 0.929 | 0.985 | 1.66E−38 | 4.17E−88 |
| Csnk2b | 2.66E−67 | −0.32158462 | 0.177 | 0.765 | 4.16E−63 | 4.77E−88 |
| Fundc2 | 1.99E−83 | −0.44850502 | 0.076 | 0.652 | 3.11E−79 | 5.97E−88 |
| Mrpl23 | 5.30E−59 | −0.28089941 | 0.06 | 0.515 | 8.27E−55 | 6.68E−88 |
| Tma7 | 1.13E−39 | −0.35076999 | 0.349 | 0.882 | 1.76E−35 | 7.08E−88 |
| Uqcrh | 2.52E−57 | −0.46051237 | 0.455 | 0.921 | 3.93E−53 | 1.45E−87 |
| Cotl1 | 4.33E−55 | −0.45674647 | 0.287 | 0.848 | 6.75E−51 | 1.70E−87 |
| Hnrnpa1 | 5.41E−67 | −0.57330597 | 0.274 | 0.841 | 8.44E−63 | 2.12E−87 |
| Gmfg | 1.29E−48 | −0.39031827 | 0.308 | 0.846 | 2.01E−44 | 2.91E−87 |
| Ndufa12 | 5.26E−69 | −0.41277513 | 0.062 | 0.564 | 8.21E−65 | 4.75E−87 |
| Rps27 | 1.19E−55 | −0.50290095 | 0.837 | 0.965 | 1.86E−51 | 7.25E−87 |
| Dbi | 2.34E−71 | −0.4141928 | 0.103 | 0.647 | 3.65E−67 | 7.57E−87 |
| H2afz | 1.11E−86 | −0.88036093 | 0.285 | 0.882 | 1.74E−82 | 1.32E−63 |
| Atp5g2 | 3.40E−75 | −0.61378443 | 0.317 | 0.899 | 5.30E−71 | 1.50E−86 |
| Psmb4 | 2.90E−59 | −0.30247754 | 0.191 | 0.757 | 4.52E−55 | 1.59E−86 |
| Uqcrq | 1.69E−46 | −0.28054211 | 0.255 | 0.794 | 2.64E−42 | 2.12E−86 |
| Eno1 | 4.17E−61 | −0.59325389 | 0.414 | 0.887 | 6.50E−57 | 2.91E−86 |
| Prdx2 | 4.41E−61 | −0.31613808 | 0.149 | 0.691 | 6.88E−57 | 3.33E−86 |
| Hspe1 | 7.85E−64 | −0.5694091 | 0.303 | 0.857 | 1.22E−59 | 3.77E−86 |
| Ube2m | 1.56E−60 | −0.26849353 | 0.147 | 0.69 | 2.43E−56 | 6.16E−86 |
| Sepw1 | 6.48E−58 | −0.36056633 | 0.228 | 0.813 | 1.01E−53 | 9.09E−86 |
| Fam96a | 6.28E−78 | −0.3839324 | 0.057 | 0.598 | 9.79E−74 | 1.68E−85 |
| Hnrnpc | 9.29E−69 | −0.43994682 | 0.175 | 0.76 | 1.45E−64 | 1.81E−85 |
| Ppp1r14b | 4.05E−64 | −0.40519571 | 0.08 | 0.574 | 6.31E−60 | 1.90E−85 |
| Sumo1 | 1.75E−83 | −0.43782225 | 0.138 | 0.765 | 2.73E−79 | 2.13E−85 |
| Mrps16 | 2.02E−65 | −0.29548642 | 0.163 | 0.742 | 3.14E−61 | 2.23E−85 |
| U2af1 | 4.42E−69 | −0.31632056 | 0.106 | 0.652 | 6.90E−65 | 3.30E−85 |
| Grpel1 | 1.42E−72 | −0.37868534 | 0.044 | 0.548 | 2.21E−68 | 4.96E−85 |
| Nudt21 | 9.60E−65 | −0.25971836 | 0.122 | 0.667 | 1.50E−60 | 5.11E−85 |
| Ubl5 | 1.05E−65 | −0.56585459 | 0.322 | 0.885 | 1.64E−61 | 9.11E−85 |
| Sec61g | 6.53E−59 | −0.43570931 | 0.315 | 0.872 | 1.02E−54 | 9.73E−85 |
| Ywhah | 4.18E−58 | −0.26828203 | 0.149 | 0.683 | 6.52E−54 | 1.28E−84 |
| Arf4 | 1.38E−73 | −0.30589704 | 0.097 | 0.655 | 2.15E−69 | 1.41E−84 |
| Fam162a | 2.32E−61 | −0.40547029 | 0.115 | 0.621 | 3.61E−57 | 1.45E−84 |
| Ube2a | 1.72E−84 | −0.42522677 | 0.051 | 0.609 | 2.69E−80 | 1.10E−83 |
| Tmsb10 | 1.42E−47 | −0.45758108 | 0.802 | 0.96 | 2.22E−43 | 1.99E−84 |
| Ywhae | 1.61E−73 | −0.41608256 | 0.166 | 0.764 | 2.51E−69 | 2.13E−84 |
| Epsti1 | 2.22E−49 | −0.29457079 | 0.251 | 0.786 | 3.47E−45 | 2.43E−84 |
| Ubald2 | 5.31E−58 | −0.27571412 | 0.14 | 0.657 | 8.28E−54 | 2.91E−84 |
| Psme1 | 4.41E−52 | −0.3820695 | 0.297 | 0.862 | 6.88E−48 | 3.99E−84 |
| Eef1g | 2.78E−56 | −0.51402295 | 0.453 | 0.922 | 4.34E−52 | 4.66E−84 |
| Rnf7 | 2.80E−65 | −0.27241055 | 0.097 | 0.619 | 4.36E−61 | 4.90E−84 |
| Ranbp1 | 1.03E−77 | −0.62981532 | 0.083 | 0.63 | 1.60E−73 | 1.01E−83 |
| Ndufa1 | 5.46E−61 | −0.26743279 | 0.177 | 0.746 | 8.52E−57 | 1.73E−83 |
| Atp5e | 5.95E−33 | −0.29528753 | 0.48 | 0.908 | 9.28E−29 | 1.75E−83 |
| Ndufs5 | 7.80E−80 | −0.40136471 | 0.064 | 0.616 | 1.22E−75 | 2.46E−83 |
| Atp5k | 6.58E−53 | −0.27445994 | 0.2 | 0.75 | 1.03E−48 | 5.94E−83 |
| Clic1 | 9.38E−80 | −0.70946785 | 0.379 | 0.944 | 1.46E−75 | 6.85E−83 |
| Pycard | 1.02E−67 | −0.41477587 | 0.136 | 0.692 | 1.59E−63 | 8.65E−83 |
| Dad1 | 1.72E−55 | −0.43907249 | 0.303 | 0.894 | 2.68E−51 | 1.42E−82 |
| Cmpk1 | 1.50E−69 | −0.27245452 | 0.106 | 0.656 | 2.33E−65 | 2.14E−82 |
| Commd3 | 9.07E−71 | −0.28841377 | 0.067 | 0.585 | 1.42E−66 | 2.31E−82 |
| Mien1 | 2.22E−81 | −0.38573414 | 0.09 | 0.672 | 3.46E−77 | 2.85E−82 |
| Hnrnpab | 3.11E−66 | −0.51403177 | 0.159 | 0.712 | 4.85E−62 | 3.41E−82 |
| Gm10076 | 5.32E−47 | −0.34739396 | 0.283 | 0.833 | 8.30E−43 | 3.74E−82 |
| Ostc | 4.09E−75 | −0.39578517 | 0.161 | 0.766 | 6.38E−71 | 4.33E−82 |
| Akr1a1 | 1.15E−75 | −0.40266317 | 0.131 | 0.72 | 1.79E−71 | 4.34E−82 |
| Polr2g | 1.26E−74 | −0.34550536 | 0.062 | 0.59 | 1.96E−70 | 5.29E−82 |
| Crip1 | 3.85E−37 | −0.44266099 | 0.4 | 0.859 | 6.01E−33 | 6.79E−82 |
| Rap1a | 9.64E−82 | −0.49795052 | 0.198 | 0.835 | 1.50E−77 | 3.32E−78 |
| Nol7 | 4.65E−50 | −0.2565218 | 0.237 | 0.789 | 7.26E−46 | 1.59E−81 |
| Ndfip1 | 1.23E−68 | −0.368348 | 0.166 | 0.756 | 1.91E−64 | 2.25E−81 |
| Cct5 | 3.58E−52 | −0.25717402 | 0.198 | 0.739 | 5.59E−48 | 2.31E−81 |
| Rpl26 | 2.26E−33 | −0.29705512 | 0.864 | 0.967 | 3.53E−29 | 2.90E−81 |
| Glrx5 | 4.19E−64 | −0.32012952 | 0.051 | 0.523 | 6.53E−60 | 3.09E−81 |
| 2700094K13Rik | 5.52E−51 | −0.29312407 | 0.103 | 0.553 | 8.61E−47 | 5.16E−81 |
| Actg1 | 1.37E−72 | −0.54946764 | 0.864 | 0.991 | 2.13E−68 | 9.00E−81 |
| Nhp2 | 7.63E−54 | −0.25724887 | 0.078 | 0.525 | 1.19E−49 | 1.02E−80 |
| Anp32e | 1.39E−51 | −0.27005101 | 0.124 | 0.601 | 2.16E−47 | 1.23E−80 |
| Lsm6 | 3.36E−62 | −0.29835559 | 0.099 | 0.606 | 5.24E−58 | 3.08E−80 |
| Abracl | 1.97E−63 | −0.48320049 | 0.29 | 0.866 | 3.07E−59 | 4.50E−80 |
| Arpc2 | 5.85E−61 | −0.52988127 | 0.536 | 0.931 | 9.13E−57 | 5.25E−80 |
| Polr2k | 5.09E−70 | −0.29122407 | 0.064 | 0.578 | 7.95E−66 | 1.15E−79 |
| 7-Sep | 2.28E−52 | −0.33970686 | 0.257 | 0.815 | 3.56E−48 | 2.23E−79 |
| Ech1 | 1.12E−65 | −0.28560255 | 0.087 | 0.6 | 1.75E−61 | 2.30E−79 |
| Ndufb2 | 1.79E−66 | −0.27767489 | 0.064 | 0.563 | 2.79E−62 | 2.46E−79 |
| Timm17a | 9.20E−70 | −0.34364151 | 0.048 | 0.545 | 1.43E−65 | 3.19E−79 |
| Psma4 | 6.27E−67 | −0.37555352 | 0.161 | 0.735 | 9.78E−63 | 3.84E−79 |
| Ybx1 | 3.52E−56 | −0.5378119 | 0.384 | 0.889 | 5.50E−52 | 5.33E−79 |
| Mrpl18 | 2.15E−67 | −0.35457983 | 0.11 | 0.645 | 3.36E−63 | 8.21E−79 |
| Actr10 | 4.78E−63 | −0.2625628 | 0.087 | 0.59 | 7.45E−59 | 8.60E−79 |
| Mcts1 | 3.39E−66 | −0.29852728 | 0.039 | 0.512 | 5.29E−62 | 1.56E−78 |
| Tubb4b | 1.68E−70 | −0.5385614 | 0.078 | 0.595 | 2.63E−66 | 1.66E−78 |
| Myl6 | 5.60E−57 | −0.52168647 | 0.637 | 0.938 | 8.74E−53 | 2.83E−78 |
| Uqcr10 | 7.84E−58 | −0.31487571 | 0.189 | 0.748 | 1.22E−53 | 3.74E−78 |
| Rnaseh2c | 2.29E−60 | −0.30583482 | 0.053 | 0.51 | 3.57E−56 | 5.09E−78 |
| Tceb2 | 7.15E−52 | −0.37588336 | 0.29 | 0.854 | 1.12E−47 | 6.33E−78 |
| Cox6b1 | 1.96E−62 | −0.49467021 | 0.285 | 0.862 | 3.06E−58 | 6.67E−78 |
| Bsg | 2.99E−61 | −0.55364271 | 0.257 | 0.834 | 4.67E−57 | 2.03E−77 |
| Romo1 | 5.37E−62 | −0.25807676 | 0.08 | 0.57 | 8.38E−58 | 2.50E−77 |
| Atp5j2 | 2.65E−37 | −0.25381546 | 0.333 | 0.855 | 4.14E−33 | 3.04E−77 |
| Rpl36al | 4.81E−53 | −0.47395425 | 0.515 | 0.933 | 7.50E−49 | 6.75E−77 |
| Cox7c | 4.26E−60 | −0.51760011 | 0.37 | 0.903 | 6.65E−56 | 7.79E−77 |
| Bcl2a1b | 8.45E−73 | −0.70881005 | 0.255 | 0.816 | 1.32E−68 | 2.12E−76 |
| Eif3h | 5.40E−53 | −0.49016915 | 0.421 | 0.913 | 8.43E−49 | 2.94E−76 |
| H2afv | 1.34E−54 | −0.30886366 | 0.147 | 0.649 | 2.10E−50 | 9.66E−76 |
| Prmt1 | 1.40E−55 | −0.25613704 | 0.062 | 0.503 | 2.19E−51 | 1.05E−75 |
| Ppib | 1.66E−53 | −0.5046958 | 0.377 | 0.929 | 2.59E−49 | 1.95E−75 |
| Rap1b | 1.25E−42 | −0.30447899 | 0.317 | 0.858 | 1.95E−38 | 7.31E−75 |
| Clta | 2.55E−41 | −0.32212816 | 0.329 | 0.865 | 3.97E−37 | 2.37E−74 |
| Prkar1a | 1.64E−46 | −0.34218624 | 0.287 | 0.837 | 2.56E−42 | 1.10E−73 |
| Arpc4 | 1.09E−51 | −0.28795751 | 0.241 | 0.807 | 1.70E−47 | 1.48E−73 |
| Ftl1 | 5.09E−64 | −0.57583847 | 0.428 | 0.929 | 7.94E−60 | 1.94E−73 |
| Pa2g4 | 3.82E−56 | −0.3273445 | 0.076 | 0.532 | 5.96E−52 | 1.98E−73 |
| Ddx39 | 1.24E−62 | −0.36072063 | 0.048 | 0.51 | 1.94E−58 | 2.17E−73 |
| Uba52 | 1.46E−35 | −0.38695267 | 0.513 | 0.914 | 2.28E−31 | 5.06E−73 |
| Rexo2 | 6.84E−57 | −0.32774502 | 0.046 | 0.474 | 1.07E−52 | 1.38E−72 |
| Mrpl51 | 5.20E−62 | −0.29028446 | 0.041 | 0.497 | 8.11E−58 | 2.67E−72 |
| Serf2 | 8.06E−41 | −0.36762734 | 0.513 | 0.921 | 1.26E−36 | 1.91E−71 |
| Sh3bgrl3 | 2.43E−49 | −0.47525599 | 0.522 | 0.919 | 3.80E−45 | 2.28E−71 |
| Atp5h | 9.06E−50 | −0.41228407 | 0.423 | 0.903 | 1.41E−45 | 2.41E−71 |
| Dynll1 | 1.85E−60 | −0.44841652 | 0.257 | 0.824 | 2.88E−56 | 2.91E−71 |
| Cdk4 | 4.64E−58 | −0.32221453 | 0.039 | 0.472 | 7.23E−54 | 2.96E−71 |
| Hmgn2 | 1.89E−58 | −0.47746015 | 0.048 | 0.487 | 2.94E−54 | 4.98E−71 |
| Ywhaz | 1.41E−50 | −0.36803733 | 0.283 | 0.844 | 2.20E−46 | 7.15E−71 |
| Nap1l1 | 7.97E−71 | −0.50382055 | 0.177 | 0.755 | 1.24E−66 | 4.33E−65 |
| Polr3k | 2.94E−57 | −0.32140437 | 0.06 | 0.507 | 4.58E−53 | 1.19E−70 |
| C1qbp | 1.11E−56 | −0.37743694 | 0.053 | 0.489 | 1.73E−52 | 1.37E−70 |
| Higd1a | 7.57E−53 | −0.34895213 | 0.237 | 0.79 | 1.18E−48 | 1.53E−70 |
| Sub1 | 1.08E−56 | −0.5811595 | 0.593 | 0.937 | 1.68E−52 | 1.73E−70 |
| Ebna1bp2 | 1.26E−55 | −0.28170838 | 0.051 | 0.482 | 1.97E−51 | 2.17E−70 |
| Pgk1 | 2.95E−70 | −0.41769343 | 0.101 | 0.639 | 4.61E−66 | 2.69E−67 |
| Rpl31 | 1.94E−36 | −0.35905787 | 0.513 | 0.913 | 3.03E−32 | 5.16E−70 |
| Cdc42 | 8.02E−61 | −0.55089696 | 0.359 | 0.909 | 1.25E−56 | 7.67E−70 |
| Dut | 9.38E−54 | −0.47958146 | 0.14 | 0.612 | 1.46E−49 | 1.16E−69 |
| Vasp | 5.62E−42 | −0.26333504 | 0.303 | 0.847 | 8.77E−38 | 1.21E−69 |
| Anxa2 | 2.66E−57 | −0.54290149 | 0.329 | 0.821 | 4.14E−53 | 1.66E−69 |
| Cd3g | 2.95E−63 | −0.5955356 | 0.607 | 0.972 | 4.60E−59 | 1.88E−69 |
| Mrpl36 | 4.81E−62 | −0.3151899 | 0.041 | 0.495 | 7.51E−58 | 2.30E−69 |
| Rhoa | 9.65E−44 | −0.3206292 | 0.349 | 0.892 | 1.51E−39 | 2.56E−69 |
| Alyref | 2.64E−59 | −0.34655175 | 0.074 | 0.542 | 4.12E−55 | 4.69E−69 |
| Rpl41 | 1.42E−38 | −0.31662911 | 0.899 | 0.973 | 2.21E−34 | 6.56E−69 |
| Psmb8 | 6.16E−38 | −0.35437223 | 0.446 | 0.902 | 9.61E−34 | 1.26E−68 |
| Hmgb1 | 5.24E−41 | −0.42468811 | 0.421 | 0.898 | 8.17E−37 | 4.40E−68 |
| Hnrnpf | 8.58E−53 | −0.40606632 | 0.347 | 0.902 | 1.34E−48 | 5.82E−68 |
| Gnai2 | 7.24E−44 | −0.36359166 | 0.377 | 0.889 | 1.13E−39 | 1.74E−67 |
| Aldoa | 4.03E−53 | −0.53416084 | 0.51 | 0.913 | 6.29E−49 | 1.83E−67 |
| Cmtm7 | 2.46E−67 | −0.42461498 | 0.094 | 0.614 | 3.84E−63 | 4.88E−62 |
| Arl6ip1 | 1.97E−44 | −0.41762686 | 0.354 | 0.887 | 3.07E−40 | 5.54E−67 |
| Cox17 | 1.67E−51 | −0.31532022 | 0.246 | 0.782 | 2.60E−47 | 1.53E−66 |
| Anp32b | 7.41E−43 | −0.41046576 | 0.368 | 0.868 | 1.16E−38 | 4.18E−66 |
| Atp5b | 1.95E−44 | −0.35118396 | 0.343 | 0.863 | 3.04E−40 | 9.73E−66 |
| Bcl2a1d | 2.79E−65 | −0.65640848 | 0.225 | 0.763 | 4.35E−61 | 9.97E−66 |
| Arf5 | 4.02E−36 | −0.30152946 | 0.395 | 0.911 | 6.27E−32 | 1.81E−65 |
| Itm2b | 6.13E−62 | −0.50152058 | 0.336 | 0.932 | 9.57E−58 | 1.63E−64 |
| H3f3b | 1.23E−54 | −0.48927395 | 0.717 | 0.963 | 1.92E−50 | 2.08E−64 |
| Hnrnpa0 | 2.58E−47 | −0.28866587 | 0.294 | 0.862 | 4.02E−43 | 2.33E−64 |
| Timm8b | 2.36E−57 | −0.28702849 | 0.041 | 0.473 | 3.68E−53 | 2.86E−64 |
| Pcbp2 | 7.37E−58 | −0.37997719 | 0.26 | 0.845 | 1.15E−53 | 1.70E−63 |
| Prelid1 | 3.20E−44 | −0.31253075 | 0.297 | 0.821 | 4.99E−40 | 1.07E−62 |
| Ostf1 | 4.19E−42 | −0.40290722 | 0.434 | 0.902 | 6.54E−38 | 3.10E−62 |
| Tuba1b | 7.56E−41 | −0.48985343 | 0.152 | 0.556 | 1.18E−36 | 4.04E−62 |
| Set | 2.51E−47 | −0.35327912 | 0.306 | 0.842 | 3.92E−43 | 5.97E−62 |
| Ppp1ca | 2.35E−37 | −0.32538243 | 0.363 | 0.859 | 3.66E−33 | 7.78E−62 |
| Plac8 | 7.48E−47 | −0.71406029 | 0.294 | 0.718 | 1.17E−42 | 3.92E−61 |
| Arpc5 | 4.73E−36 | −0.30071157 | 0.349 | 0.852 | 7.38E−32 | 4.25E−61 |
| Nkg7 | 6.28E−61 | −0.62825154 | 0.77 | 0.983 | 9.80E−57 | 1.75E−54 |
| Glipr1 | 2.57E−53 | −0.30643181 | 0.08 | 0.522 | 4.01E−49 | 1.03E−60 |
| Eif3f | 4.51E−43 | −0.39935568 | 0.46 | 0.915 | 7.04E−39 | 3.06E−59 |
| Ldha | 4.38E−47 | −0.48389256 | 0.579 | 0.933 | 6.83E−43 | 2.55E−58 |
| Ybx3 | 1.19E−37 | −0.39628911 | 0.379 | 0.831 | 1.86E−33 | 7.34E−56 |
| Ly6a | 1.44E−38 | −0.48171228 | 0.384 | 0.814 | 2.25E−34 | 2.10E−55 |
| Tubb5 | 7.07E−35 | −0.52351753 | 0.331 | 0.779 | 1.10E−30 | 4.57E−55 |
| Rac2 | 5.35E−31 | −0.31679617 | 0.616 | 0.933 | 8.35E−27 | 2.09E−53 |
| AW112010 | 6.94E−53 | −0.54750442 | 0.678 | 0.968 | 1.08E−48 | 2.90E−52 |
| Serpinb6b | 2.81E−37 | −0.46534975 | 0.423 | 0.827 | 4.39E−33 | 2.05E−51 |
| Gapdh | 1.32E−37 | −0.41211304 | 0.795 | 0.953 | 2.06E−33 | 7.40E−51 |
| Slc25a3 | 2.99E−33 | −0.29820451 | 0.441 | 0.911 | 4.67E−29 | 1.02E−49 |
| mt-Nd4l | 8.33E−33 | 0.973238245 | 0.701 | 0.829 | 1.30E−28 | 1.58E−49 |
| Cd52 | 8.22E−45 | −0.49879431 | 0.782 | 0.979 | 1.28E−40 | 1.67E−49 |
| Serbp1 | 1.03E−44 | −0.42370995 | 0.464 | 0.927 | 1.61E−40 | 2.47E−48 |
| Arpc1b | 1.18E−27 | −0.29435134 | 0.506 | 0.913 | 1.83E−23 | 5.33E−48 |
| Cox4i1 | 2.54E−27 | −0.27542828 | 0.566 | 0.935 | 3.96E−23 | 1.68E−47 |
| Klf2 | 2.93E−23 | −0.25853176 | 0.189 | 0.503 | 4.58E−19 | 2.00E−47 |
| Cyba | 2.77E−45 | −0.48006136 | 0.432 | 0.938 | 4.33E−41 | 8.04E−47 |
| H2afx | 2.80E−37 | −0.32944898 | 0.023 | 0.327 | 4.37E−33 | 1.69E−46 |
| Cd53 | 2.69E−46 | −0.40308704 | 0.437 | 0.959 | 4.19E−42 | 1.83E−42 |
| Actr3 | 7.88E−45 | −0.37751521 | 0.409 | 0.927 | 1.23E−40 | 1.95E−45 |
| S100a4 | 1.02E−33 | −0.62222866 | 0.457 | 0.785 | 1.59E−29 | 4.99E−45 |
| Cd8b1 | 3.11E−23 | −0.32127204 | 0.561 | 0.908 | 4.85E−19 | 1.08E−43 |
| Gadd45b | 2.17E−29 | −0.29645133 | 0.193 | 0.554 | 3.39E−25 | 6.65E−43 |
| Calr | 4.46E−41 | −0.38008836 | 0.347 | 0.863 | 6.95E−37 | 4.32E−41 |
| Vim | 3.64E−19 | −0.28581539 | 0.54 | 0.842 | 5.67E−15 | 2.07E−40 |
| Ifng | 2.30E−40 | −1.18468175 | 0.248 | 0.688 | 3.58E−36 | 1.46E−37 |
| Hmgb2 | 3.46E−37 | −0.6306558 | 0.405 | 0.855 | 5.41E−33 | 8.41E−40 |
| Tnfrsf9 | 5.34E−27 | −0.3812789 | 0.441 | 0.819 | 8.33E−23 | 8.70E−40 |
| Gzmb | 1.54E−32 | −1.00411457 | 0.722 | 0.918 | 2.41E−28 | 1.63E−38 |
| Lgals1 | 7.74E−27 | −0.37069766 | 0.692 | 0.907 | 1.21E−22 | 7.25E−38 |
| Prf1 | 1.23E−36 | −0.410947 | 0.223 | 0.643 | 1.91E−32 | 1.25E−36 |
| Stmn1 | 1.47E−32 | −0.72973351 | 0.053 | 0.341 | 2.30E−28 | 1.65E−35 |
| S100a6 | 3.05E−27 | −0.47508228 | 0.667 | 0.912 | 4.76E−23 | 6.55E−33 |
| Tnfrsf4 | 1.69E−24 | −0.3714802 | 0.207 | 0.532 | 2.64E−20 | 2.36E−30 |
| Top2a | 1.06E−29 | −0.72077386 | 0.057 | 0.326 | 1.65E−25 | 3.57E−24 |
| Ggnbp2 | 2.71E−06 | 0.406954306 | 0.322 | 0.64 | 0.042282 | 3.73E−27 |
| Srrm2 | 9.36E−14 | 0.517739739 | 0.685 | 0.926 | 1.46E−09 | 4.47E−27 |
| Ptprc | 7.22E−16 | 0.355472277 | 0.897 | 0.993 | 1.13E−11 | 6.20E−27 |
| Ifitm2 | 1.94E−26 | −0.64080993 | 0.175 | 0.469 | 3.02E−22 | 1.86E−19 |
| Kansl1 | 2.94E−18 | 0.313906927 | 0.209 | 0.551 | 4.58E−14 | 5.89E−26 |
| Kdm2a | 7.78E−18 | 0.263544776 | 0.223 | 0.573 | 1.21E−13 | 6.70E−26 |
| Sbno1 | 1.02E−10 | 0.354278387 | 0.315 | 0.686 | 1.59E−06 | 1.57E−25 |
| Hsph1 | 2.00E−12 | 0.328926457 | 0.239 | 0.539 | 3.12E−08 | 3.03E−25 |
| Dhx36 | 5.27E−11 | 0.344419612 | 0.276 | 0.612 | 8.22E−07 | 6.33E−25 |
| Gzma | 8.70E−24 | −0.87414617 | 0.237 | 0.525 | 1.36E−19 | 1.15E−19 |
| Laptm5 | 8.78E−24 | 0.520834379 | 0.832 | 0.98 | 1.37E−19 | 1.55E−23 |
| Rbbp6 | 7.96E−18 | 0.273028951 | 0.251 | 0.632 | 1.24E−13 | 9.75E−24 |
| Ddx46 | 6.05E−11 | 0.319748369 | 0.31 | 0.676 | 9.43E−07 | 1.43E−23 |
| Ccl3 | 2.17E−22 | −1.29925669 | 0.207 | 0.492 | 3.38E−18 | 1.50E−23 |
| Brd1 | 9.99E−18 | 0.289441691 | 0.218 | 0.566 | 1.56E−13 | 2.32E−23 |
| Sltm | 1.16E−14 | 0.297656076 | 0.246 | 0.591 | 1.81E−10 | 3.40E−23 |
| Cdkn1b | 4.81E−14 | 0.266556905 | 0.239 | 0.565 | 7.50E−10 | 3.42E−23 |
| 2810417H13Rik | 4.79E−23 | −0.60276967 | 0.039 | 0.253 | 7.47E−19 | 4.45E−20 |
| Rasal3 | 4.71E−11 | 0.375592359 | 0.26 | 0.58 | 7.35E−07 | 7.09E−23 |
| Txk | 6.60E−12 | 0.322630136 | 0.232 | 0.52 | 1.03E−07 | 9.52E−23 |
| Itch | 5.12E−17 | 0.292847877 | 0.177 | 0.475 | 7.98E−13 | 1.76E−22 |
| Ubr2 | 9.80E−16 | 0.305855978 | 0.186 | 0.479 | 1.53E−11 | 2.52E−22 |
| Jmjd1c | 3.78E−09 | 0.412146863 | 0.248 | 0.524 | 5.89E−05 | 3.39E−22 |
| Krit1 | 2.38E−14 | 0.350876665 | 0.274 | 0.638 | 3.71E−10 | 3.85E−22 |
| R3hdm1 | 1.61E−11 | 0.347211883 | 0.274 | 0.609 | 2.51E−07 | 4.67E−22 |
| Nrd1 | 6.01E−18 | 0.26310535 | 0.223 | 0.576 | 9.37E−14 | 5.92E−22 |
| Acap1 | 2.71E−10 | 0.373447983 | 0.239 | 0.523 | 4.23E−06 | 7.63E−22 |
| Zc3h13 | 1.35E−14 | 0.30297525 | 0.237 | 0.568 | 2.10E−10 | 1.46E−21 |
| Zfp638 | 1.16E−12 | 0.327800595 | 0.2 | 0.474 | 1.81E−08 | 1.77E−21 |
| Setd2 | 5.55E−12 | 0.332245612 | 0.207 | 0.479 | 8.65E−08 | 4.46E−21 |
| Hist1h2ap | 1.80E−17 | −0.71003269 | 0.067 | 0.253 | 2.80E−13 | 4.60E−21 |
| Itsn2 | 4.08E−16 | 0.250389765 | 0.186 | 0.484 | 6.37E−12 | 6.11E−21 |
| Xcl1 | 1.74E−11 | −0.37177863 | 0.044 | 0.17 | 2.71E−07 | 6.16E−21 |
| Ubr5 | 1.46E−11 | 0.299016774 | 0.269 | 0.597 | 2.28E−07 | 1.34E−20 |
| Wapl | 2.04E−14 | 0.278819136 | 0.287 | 0.678 | 3.19E−10 | 1.81E−20 |
| Cdk12 | 6.06E−09 | 0.41629108 | 0.255 | 0.539 | 9.46E−05 | 2.13E−20 |
| Phf14 | 4.53E−20 | 0.261129492 | 0.2 | 0.546 | 7.07E−16 | 2.16E−20 |
| Hnrnph1 | 6.51E−20 | 0.26993628 | 0.26 | 0.67 | 1.01E−15 | 2.88E−20 |
| Rbm39 | 2.90E−20 | 0.508528849 | 0.802 | 0.97 | 4.53E−16 | 1.86E−19 |
| Suco | 8.32E−09 | 0.396607849 | 0.223 | 0.469 | 0.00013 | 3.08E−20 |
| Ivns1abp | 1.83E−11 | 0.346901684 | 0.184 | 0.429 | 2.85E−07 | 3.87E−20 |
| Chd8 | 1.04E−17 | 0.275932754 | 0.179 | 0.485 | 1.62E−13 | 9.20E−20 |
| Dync1h1 | 1.79E−11 | 0.311955257 | 0.175 | 0.412 | 2.79E−07 | 9.29E−20 |
| Tcf12 | 2.14E−16 | 0.329573029 | 0.163 | 0.439 | 3.34E−12 | 9.77E−20 |
| Zfp644 | 1.53E−08 | 0.451469865 | 0.246 | 0.514 | 0.000239 | 1.50E−19 |
| Dennd4a | 1.33E−15 | 0.312034021 | 0.232 | 0.565 | 2.07E−11 | 1.69E−19 |
| Huwe1 | 2.20E−14 | 0.281150969 | 0.241 | 0.578 | 3.43E−10 | 1.98E−19 |
| Arid4a | 3.20E−15 | 0.267000188 | 0.244 | 0.585 | 4.99E−11 | 2.07E−19 |
| Herc1 | 1.85E−06 | 0.445621225 | 0.269 | 0.527 | 0.028936 | 2.90E−19 |
| Arglu1 | 0.000153 | 0.448567226 | 0.347 | 0.666 | 1 | 5.54E−19 |
| Snrnp70 | 2.22E−12 | 0.326647116 | 0.287 | 0.651 | 3.46E−08 | 6.64E−19 |
| Parp14 | 7.62E−08 | 0.397977132 | 0.195 | 0.403 | 0.001188 | 6.66E−19 |
| Fnbp4 | 2.55E−10 | 0.393614593 | 0.205 | 0.457 | 3.98E−06 | 6.85E−19 |
| Raf1 | 7.08E−19 | 0.290783453 | 0.17 | 0.476 | 1.10E−14 | 7.94E−19 |
| Jun | 4.66E−12 | 0.353170092 | 0.172 | 0.404 | 7.27E−08 | 8.07E−19 |
| Hnrnpl | 2.56E−08 | 0.390821633 | 0.313 | 0.656 | 0.0004 | 9.03E−19 |
| Usp48 | 4.23E−08 | 0.430202385 | 0.251 | 0.517 | 0.00066 | 1.04E−18 |
| Pstpip1 | 9.18E−05 | 0.385079435 | 0.37 | 0.714 | 1 | 1.25E−18 |
| Srrt | 7.04E−14 | 0.309745719 | 0.193 | 0.474 | 1.10E−09 | 1.33E−18 |
| Clcn3 | 2.80E−17 | 0.324804711 | 0.191 | 0.501 | 4.38E−13 | 1.36E−18 |
| Atp11b | 1.39E−18 | 0.27042968 | 0.264 | 0.66 | 2.17E−14 | 1.64E−16 |
| Ranbp2 | 4.22E−11 | 0.324496517 | 0.241 | 0.537 | 6.58E−07 | 2.45E−18 |
| Mkln1 | 1.35E−13 | 0.377497391 | 0.198 | 0.478 | 2.11E−09 | 3.53E−18 |
| Gpr132 | 1.62E−14 | 0.353860686 | 0.189 | 0.469 | 2.52E−10 | 4.06E−18 |
| Tia1 | 1.13E−12 | 0.277569143 | 0.179 | 0.432 | 1.77E−08 | 4.55E−18 |
| Rif1 | 5.84E−18 | 0.283139754 | 0.195 | 0.518 | 9.11E−14 | 9.52E−12 |
| Zfc3h1 | 1.28E−13 | 0.296662728 | 0.14 | 0.37 | 2.00E−09 | 6.58E−18 |
| Slc1a5 | 1.63E−12 | 0.29530046 | 0.269 | 0.608 | 2.54E−08 | 6.82E−18 |
| Ssh2 | 1.39E−10 | 0.303019992 | 0.228 | 0.491 | 2.17E−06 | 7.14E−18 |
| Ogt | 1.28E−08 | 0.349048845 | 0.356 | 0.741 | 0.0002 | 7.63E−18 |
| Phf20l1 | 2.05E−07 | 0.450974468 | 0.294 | 0.596 | 0.003206 | 7.83E−18 |
| Map4k4 | 2.77E−11 | 0.383618577 | 0.26 | 0.575 | 4.32E−07 | 9.61E−18 |
| Asxl2 | 8.42E−16 | 0.255599297 | 0.156 | 0.422 | 1.31E−11 | 1.06E−17 |
| Tet2 | 1.21E−06 | 0.431782931 | 0.209 | 0.412 | 0.018823 | 1.20E−17 |
| Pan3 | 4.67E−06 | 0.455171904 | 0.191 | 0.37 | 0.072914 | 1.41E−17 |
| Nufip2 | 7.23E−10 | 0.376006543 | 0.241 | 0.521 | 1.13E−05 | 1.45E−17 |
| Hdac7 | 2.81E−07 | 0.434278524 | 0.257 | 0.518 | 0.004384 | 1.52E−17 |
| Prrc2a | 1.71E−13 | 0.39613781 | 0.246 | 0.58 | 2.67E−09 | 2.04E−17 |
| Gm26917 | 2.35E−17 | 1.354435767 | 0.584 | 0.697 | 3.67E−13 | 4.08E−05 |
| Pde7a | 7.84E−08 | 0.469636992 | 0.28 | 0.567 | 0.001224 | 2.66E−17 |
| Trpm7 | 3.64E−17 | 0.256792509 | 0.179 | 0.478 | 5.67E−13 | 1.08E−16 |
| Gcc2 | 3.85E−17 | 0.256804824 | 0.133 | 0.39 | 6.01E−13 | 8.64E−16 |
| Ccnt2 | 6.77E−13 | 0.383236403 | 0.161 | 0.401 | 1.06E−08 | 4.30E−17 |
| Rasa1 | 2.28E−16 | 0.253939584 | 0.186 | 0.486 | 3.55E−12 | 5.39E−17 |
| Upf2 | 3.54E−15 | 0.267299793 | 0.133 | 0.372 | 5.52E−11 | 6.14E−17 |
| Atf7ip | 1.84E−08 | 0.44651149 | 0.267 | 0.549 | 0.000288 | 7.50E−17 |
| Ttc3 | 9.84E−13 | 0.272091683 | 0.14 | 0.36 | 1.54E−08 | 7.83E−17 |
| Nfat5 | 9.66E−08 | 0.37535272 | 0.292 | 0.585 | 0.001507 | 1.10E−16 |
| Trmt1l | 1.16E−10 | 0.252745601 | 0.147 | 0.349 | 1.81E−06 | 1.12E−16 |
| Adgre5 | 1.42E−12 | 0.264138681 | 0.241 | 0.545 | 2.22E−08 | 1.19E−16 |
| Pcf11 | 6.81E−12 | 0.334881305 | 0.191 | 0.445 | 1.06E−07 | 1.20E−16 |
| Itpkb | 1.48E−16 | 0.264980236 | 0.267 | 0.643 | 2.31E−12 | 2.76E−12 |
| Eif4g3 | 2.78E−15 | 0.332109679 | 0.253 | 0.616 | 4.34E−11 | 1.67E−16 |
| Peak1 | 1.78E−16 | 0.298636705 | 0.198 | 0.508 | 2.77E−12 | 1.03E−11 |
| Rexo1 | 1.04E−13 | 0.322434404 | 0.163 | 0.415 | 1.63E−09 | 2.50E−16 |
| Cep250 | 4.06E−11 | 0.296288725 | 0.182 | 0.42 | 6.34E−07 | 2.96E−16 |
| Tmem259 | 3.38E−12 | 0.316409109 | 0.17 | 0.411 | 5.27E−08 | 3.50E−16 |
| Pik3cd | 4.86E−06 | 0.399871334 | 0.349 | 0.693 | 0.075797 | 3.55E−16 |
| Phf3 | 3.38E−13 | 0.288787475 | 0.306 | 0.701 | 5.27E−09 | 3.81E−16 |
| Madd | 2.94E−05 | 0.402547882 | 0.306 | 0.576 | 0.45929 | 4.13E−16 |
| Tmc6 | 2.21E−08 | 0.361785372 | 0.172 | 0.366 | 0.000345 | 4.27E−16 |
| Golga4 | 4.51E−09 | 0.425829932 | 0.257 | 0.546 | 7.03E−05 | 4.78E−16 |
| Nisch | 6.55E−08 | 0.464123071 | 0.271 | 0.555 | 0.001023 | 6.88E−16 |
| Ccl4 | 3.53E−12 | −1.14574022 | 0.462 | 0.726 | 5.51E−08 | 7.11E−16 |
| Nbeal2 | 5.17E−07 | 0.458299405 | 0.218 | 0.436 | 0.008058 | 7.76E−16 |
| Dmtf1 | 6.10E−10 | 0.437488335 | 0.182 | 0.406 | 9.51E−06 | 9.68E−16 |
| Spag9 | 5.01E−15 | 0.259250167 | 0.26 | 0.616 | 7.82E−11 | 1.04E−15 |
| Slc30a9 | 1.13E−09 | 0.408907357 | 0.163 | 0.369 | 1.77E−05 | 1.04E−15 |
| Jarid2 | 1.61E−12 | 0.319448058 | 0.209 | 0.489 | 2.51E−08 | 1.12E−15 |
| Vcpip1 | 5.70E−12 | 0.295457188 | 0.12 | 0.314 | 8.89E−08 | 1.12E−15 |
| Ythdc1 | 5.97E−09 | 0.401934194 | 0.31 | 0.659 | 9.31E−05 | 1.41E−15 |
| Kif21b | 5.18E−14 | 0.296333947 | 0.175 | 0.439 | 8.08E−10 | 1.73E−15 |
| Ccl5 | 1.50E−10 | −0.40172042 | 0.736 | 0.895 | 2.35E−06 | 1.97E−15 |
| Git2 | 2.32E−15 | 0.371401886 | 0.257 | 0.618 | 3.62E−11 | 7.74E−12 |
| Hivep2 | 2.33E−15 | 0.253481334 | 0.126 | 0.361 | 3.63E−11 | 3.21E−13 |
| Zzef1 | 1.69E−10 | 0.315958047 | 0.175 | 0.398 | 2.63E−06 | 2.66E−15 |
| Tet3 | 2.76E−15 | 0.262762218 | 0.161 | 0.425 | 4.31E−11 | 1.54E−14 |
| Ikzf3 | 4.57E−15 | 0.265364203 | 0.211 | 0.52 | 7.14E−11 | 1.28E−14 |
| Ip6k1 | 1.37E−08 | 0.281604386 | 0.138 | 0.309 | 0.000214 | 4.83E−15 |
| Ankrd11 | 4.52E−13 | 0.755261388 | 0.639 | 0.877 | 7.05E−09 | 5.28E−15 |
| Chd3 | 7.32E−08 | 0.402844986 | 0.26 | 0.529 | 0.001142 | 5.97E−15 |
| Tcf25 | 1.33E−13 | 0.262650166 | 0.322 | 0.732 | 2.07E−09 | 6.06E−15 |
| Hectd1 | 1.57E−12 | 0.30335381 | 0.326 | 0.739 | 2.44E−08 | 6.98E−15 |
| Gzmc | 9.99E−15 | −0.7980156 | 0.069 | 0.237 | 1.56E−10 | 3.37E−13 |
| Arid4b | 5.30E−10 | 0.366780752 | 0.32 | 0.689 | 8.26E−06 | 1.24E−14 |
| Suv420h1 | 2.52E−11 | 0.313411157 | 0.17 | 0.4 | 3.93E−07 | 1.33E−14 |
| Paxbp1 | 1.04E−11 | 0.276837774 | 0.14 | 0.348 | 1.62E−07 | 1.34E−14 |
| Fryl | 1.60E−12 | 0.30667613 | 0.239 | 0.547 | 2.50E−08 | 1.40E−14 |
| BC005537 | 1.10E−05 | 0.359558108 | 0.347 | 0.678 | 0.171209 | 1.50E−14 |
| Dennd1c | 1.14E−05 | 0.340698977 | 0.184 | 0.348 | 0.178154 | 1.78E−14 |
| Baz2a | 1.83E−14 | 0.306143623 | 0.2 | 0.493 | 2.86E−10 | 1.12E−13 |
| Akap13 | 3.78E−10 | 0.491152595 | 0.68 | 0.935 | 5.90E−06 | 2.43E−14 |
| Atp8b4 | 1.83E−11 | 0.31568476 | 0.308 | 0.668 | 2.85E−07 | 2.50E−14 |
| Bod1l | 0.000243 | 0.461590182 | 0.349 | 0.65 | 1 | 3.29E−14 |
| Tle4 | 4.42E−14 | 0.254939239 | 0.117 | 0.331 | 6.90E−10 | 4.00E−14 |
| Fam208a | 4.22E−12 | 0.290819059 | 0.124 | 0.324 | 6.59E−08 | 5.74E−14 |
| Galnt6 | 2.37E−08 | 0.401909459 | 0.248 | 0.512 | 0.00037 | 7.79E−14 |
| Smg6 | 1.50E−09 | 0.373444367 | 0.205 | 0.444 | 2.34E−05 | 7.97E−14 |
| Sytl2 | 1.10E−13 | 0.295025136 | 0.202 | 0.486 | 1.72E−09 | 3.28E−05 |
| AU020206 | 0.000317 | 0.534816372 | 0.297 | 0.538 | 1 | 1.12E−13 |
| Thoc2 | 0.000253 | 0.505101678 | 0.363 | 0.695 | 1 | 1.19E−13 |
| Map4k2 | 6.62E−11 | 0.287104054 | 0.191 | 0.434 | 1.03E−06 | 1.25E−13 |
| Nlrc3 | 3.84E−10 | 0.312827782 | 0.163 | 0.374 | 5.99E−06 | 1.26E−13 |
| Ubn2 | 2.06E−11 | 0.409362118 | 0.23 | 0.521 | 3.22E−07 | 1.63E−13 |
| Samd9l | 2.61E−07 | 0.458744398 | 0.189 | 0.383 | 0.004064 | 2.27E−13 |
| Safb2 | 1.19E−06 | 0.405832407 | 0.308 | 0.612 | 0.018609 | 2.44E−13 |
| Mxd4 | 3.66E−13 | 0.276290865 | 0.163 | 0.405 | 5.72E−09 | 2.76E−09 |
| Pnisr | 4.11E−08 | 0.443226341 | 0.32 | 0.661 | 0.000641 | 4.02E−13 |
| Prpf38b | 0.060951 | 0.561928706 | 0.421 | 0.745 | 1 | 5.19E−13 |
| Ppp1r10 | 9.46E−13 | 0.276800413 | 0.143 | 0.364 | 1.48E−08 | 5.41E−13 |
| Son | 6.50E−13 | 0.564076838 | 0.655 | 0.911 | 1.01E−08 | 6.68E−07 |
| Slc20a1 | 4.45E−08 | 0.365804514 | 0.202 | 0.418 | 0.000695 | 6.71E−13 |
| Cdk11b | 3.75E−09 | 0.395775662 | 0.292 | 0.622 | 5.85E−05 | 9.66E−13 |
| Nsd1 | 1.00E−12 | 0.352816679 | 0.246 | 0.569 | 1.56E−08 | 3.04E−11 |
| Kdm5a | 5.03E−09 | 0.434444934 | 0.313 | 0.662 | 7.84E−05 | 1.11E−12 |
| Atp2b1 | 1.80E−06 | 0.341813334 | 0.389 | 0.767 | 0.028064 | 1.22E−12 |
| Ankrd17 | 2.61E−07 | 0.453019346 | 0.315 | 0.637 | 0.004076 | 1.43E−12 |
| Nabp1 | 6.92E−05 | 0.315017463 | 0.409 | 0.766 | 1 | 1.52E−12 |
| Fcho2 | 5.56E−12 | 0.273336328 | 0.152 | 0.373 | 8.67E−08 | 1.68E−12 |
| Rsbn1l | 3.01E−12 | 0.370687393 | 0.297 | 0.667 | 4.70E−08 | 8.12E−10 |
| Chd6 | 5.03E−11 | 0.34006825 | 0.138 | 0.337 | 7.84E−07 | 3.24E−12 |
| Smchd1 | 0.005035 | 0.38366165 | 0.418 | 0.761 | 1 | 3.25E−12 |
| Vps54 | 3.55E−12 | 0.368433289 | 0.253 | 0.573 | 5.54E−08 | 5.96E−11 |
| Grina | 1.96E−08 | 0.457465889 | 0.239 | 0.497 | 0.000306 | 5.55E−12 |
| Fus | 1.79E−09 | 0.32193767 | 0.391 | 0.824 | 2.79E−05 | 6.31E−12 |
| Golgb1 | 1.42E−06 | 0.463409991 | 0.228 | 0.446 | 0.022173 | 7.16E−12 |
| Kmt2c | 7.12E−06 | 0.447625172 | 0.202 | 0.386 | 0.111033 | 7.57E−12 |
| Zc3h7a | 8.20E−12 | 0.347354435 | 0.237 | 0.537 | 1.28E−07 | 0.0016164 |
| Rb1cc1 | 2.12E−11 | 0.342056959 | 0.262 | 0.58 | 3.31E−07 | 9.58E−12 |
| Kcnq1ot1 | 9.08E−08 | 0.29437275 | 0.241 | 0.468 | 0.001416 | 1.32E−11 |
| Ccnl2 | 0.003168 | 0.540575169 | 0.366 | 0.667 | 1 | 1.39E−11 |
| Ttc14 | 1.76E−07 | 0.32205379 | 0.347 | 0.705 | 0.002745 | 1.83E−11 |
| Gripap1 | 1.87E−09 | 0.306422895 | 0.131 | 0.308 | 2.91E−05 | 1.87E−11 |
| Smg1 | 4.87E−06 | 0.49773342 | 0.276 | 0.534 | 0.075933 | 1.92E−11 |
| Atrx | 2.59E−06 | 0.597909693 | 0.575 | 0.842 | 0.040361 | 2.10E−11 |
| Hip1 | 2.59E−11 | 0.307125454 | 0.223 | 0.5 | 4.05E−07 | 2.59E−11 |
| Mgea5 | 7.91E−07 | 0.40526075 | 0.29 | 0.575 | 0.012335 | 2.92E−11 |
| Arhgap15 | 3.24E−11 | 0.362956769 | 0.306 | 0.672 | 5.06E−07 | 1.02E−07 |
| Ktn1 | 4.98E−11 | 0.316310224 | 0.191 | 0.434 | 7.77E−07 | 3.29E−10 |
| Mbnl1 | 4.55E−09 | 0.39954865 | 0.731 | 0.949 | 7.09E−05 | 7.15E−11 |
| Gbp8 | 6.71E−05 | 0.391779124 | 0.168 | 0.309 | 1 | 7.37E−11 |
| Neurl3 | 1.07E−10 | 0.371646456 | 0.267 | 0.567 | 1.66E−06 | 4.25E−06 |
| Phip | 2.45E−08 | 0.421632644 | 0.271 | 0.557 | 0.000383 | 1.43E−10 |
| Nipbl | 6.24E−06 | 0.402347318 | 0.354 | 0.702 | 0.097277 | 2.35E−10 |
| Bcl11b | 2.98E−10 | 0.383749161 | 0.306 | 0.657 | 4.66E−06 | 9.23E−10 |
| Zcchc11 | 4.17E−10 | 0.351768246 | 0.329 | 0.692 | 6.51E−06 | 1.66E−08 |
| Dusp5 | 8.41E−06 | 0.315794965 | 0.395 | 0.733 | 0.131142 | 5.18E−10 |
| Gigyf1 | 6.12E−07 | 0.439242644 | 0.172 | 0.349 | 0.00954 | 5.21E−10 |
| Ccdc88b | 5.24E−06 | 0.461424842 | 0.209 | 0.402 | 0.081784 | 5.81E−10 |
| Rbm5 | 4.03E−06 | 0.39629133 | 0.361 | 0.718 | 0.062932 | 6.52E−10 |
| Trip11 | 2.05E−07 | 0.456362779 | 0.294 | 0.599 | 0.003198 | 8.74E−10 |
| Rrbp1 | 6.73E−06 | 0.344701595 | 0.361 | 0.698 | 0.105069 | 9.73E−10 |
| Nlrc5 | 1.38E−09 | 0.451828521 | 0.228 | 0.491 | 2.15E−05 | 1.08E−09 |
| Adgrg5 | 1.12E−09 | 0.306108466 | 0.156 | 0.354 | 1.74E−05 | 5.37E−06 |
| Clec2d | 0.586352 | 0.496722843 | 0.492 | 0.786 | 1 | 1.36E−09 |
| Plec | 1.42E−09 | 0.388832399 | 0.225 | 0.486 | 2.21E−05 | 9.06E−08 |
| Setbp1 | 1.45E−09 | 0.316744192 | 0.149 | 0.341 | 2.26E−05 | 2.25E−07 |
| Tnrc6a | 1.42E−08 | 0.475970902 | 0.271 | 0.569 | 0.000221 | 1.60E−09 |
| Gramd1a | 0.553842 | 0.643926748 | 0.375 | 0.591 | 1 | 1.80E−09 |
| Tra2a | 6.96E−08 | 0.377245078 | 0.37 | 0.758 | 0.001086 | 2.63E−09 |
| Gm26740 | 0.00685 | 0.446277147 | 0.205 | 0.324 | 1 | 2.90E−09 |
| Tra2b | 4.39E−05 | 0.374845569 | 0.414 | 0.794 | 0.684789 | 3.09E−09 |
| Tnrc6b | 9.14E−09 | 0.402335454 | 0.299 | 0.619 | 0.000143 | 3.25E−09 |
| Zcchc7 | 5.17E−05 | 0.505594135 | 0.317 | 0.606 | 0.807091 | 5.49E−09 |
| Prpf4b | 1.13E−08 | 0.460238337 | 0.31 | 0.655 | 0.000177 | 7.54E−09 |
| Vgll4 | 3.99E−08 | 0.330905561 | 0.377 | 0.763 | 0.000622 | 9.68E−09 |
| 2810474O19Rik | 0.026795 | 0.496533924 | 0.437 | 0.762 | 1 | 1.04E−08 |
| Ash1l | 1.01E−05 | 0.458565671 | 0.32 | 0.617 | 0.157178 | 1.07E−08 |
| Baz1a | 0.051583 | 0.478362983 | 0.572 | 0.831 | 1 | 1.74E−08 |
| Mirt1 | 2.15E−08 | 0.391321045 | 0.149 | 0.326 | 0.000335 | 1.39E−06 |
| Ifitm1 | 3.81E−08 | −0.53564739 | 0.324 | 0.482 | 0.000595 | 1.07E−05 |
| Rinl | 0.27124 | 0.466787895 | 0.434 | 0.746 | 1 | 3.99E−08 |
| Erdr1 | 0.250148 | 0.69280517 | 0.4 | 0.652 | 1 | 4.91E−08 |
| Akap9 | 4.75E−07 | 0.409769532 | 0.292 | 0.581 | 0.007413 | 5.10E−08 |
| Il18rap | 0.000154 | 0.426343389 | 0.372 | 0.678 | 1 | 6.21E−08 |
| Itga4 | 7.43E−08 | 0.281240992 | 0.232 | 0.454 | 0.001159 | 3.28E−06 |
| Tnrc6c | 0.001365 | 0.542707779 | 0.306 | 0.544 | 1 | 1.34E−07 |
| Cntrl | 4.95E−05 | 0.393585618 | 0.393 | 0.749 | 0.772108 | 2.84E−07 |
| Mndal | 8.39E−05 | 0.377827656 | 0.382 | 0.722 | 1 | 3.82E−07 |
| Dock2 | 0.012068 | 0.356434976 | 0.453 | 0.819 | 1 | 9.29E−07 |
| Rbm25 | 0.821913 | 0.352721112 | 0.508 | 0.846 | 1 | 1.71E−06 |
| Fyb | 1.88E−06 | 0.542459282 | 0.611 | 0.858 | 0.029313 | 0.9616142 |
| Satb1 | 2.59E−06 | 0.308698024 | 0.432 | 0.811 | 0.04044 | 0.1588182 |
| Macf1 | 0.000101 | 0.755270567 | 0.522 | 0.741 | 1 | 3.26E−06 |
| Ikzf2 | 4.63E−06 | 0.50571121 | 0.23 | 0.416 | 0.072292 | 0.063428 |
| Bptf | 0.00062 | 0.420063113 | 0.398 | 0.756 | 1 | 5.08E−06 |
| Rapgef6 | 5.84E−06 | 0.320732434 | 0.384 | 0.754 | 0.091108 | 3.63E−05 |
| PISD | 0.00612 | 0.583079828 | 0.297 | 0.5 | 1 | 6.74E−06 |
| Arap2 | 1.08E−05 | 0.415077555 | 0.37 | 0.709 | 0.168396 | 3.11E−05 |
| Neb | 1.10E−05 | 0.262333004 | 0.11 | 0.223 | 0.17109 | 0.0011273 |
| Arhgef1 | 0.023546 | 0.427779201 | 0.402 | 0.727 | 1 | 1.15E−05 |
| mt-Atp8 | 0.0704 | 0.727928551 | 0.411 | 0.569 | 1 | 1.19E−05 |
| Ahnak | 1.34E−05 | 0.688096554 | 0.582 | 0.815 | 0.209591 | 0.3571544 |
| Gzmf | 0.000162 | −0.99075365 | 0.126 | 0.211 | 1 | 1.51E−05 |
| H2-Q4 | 1.53E−05 | 0.404523942 | 0.407 | 0.781 | 0.239069 | 0.0001013 |
| Lars2 | 1.78E−05 | 1.147686228 | 0.395 | 0.455 | 0.277064 | 0.0002078 |
| Bcl2 | 1.94E−05 | 0.44251003 | 0.354 | 0.619 | 0.302106 | 1.87E−05 |
| Usp34 | 0.000101 | 0.500958092 | 0.361 | 0.693 | 1 | 2.54E−05 |
| 4932438A13Rik | 5.03E−05 | 0.513369657 | 0.333 | 0.631 | 0.784314 | 0.889755 |
| AY036118 | 0.026985 | 0.607878609 | 0.228 | 0.355 | 1 | 5.21E−05 |
| Smad7 | 6.86E−05 | 0.499138899 | 0.359 | 0.675 | 1 | 0.3640972 |
| Dgat1 | 0.013804 | 0.670665586 | 0.347 | 0.594 | 1 | 0.0002462 |
| Birc6 | 0.000631 | 0.475008837 | 0.379 | 0.722 | 1 | 0.0003094 |
| Kmt2e | 0.00031 | 0.532220839 | 0.586 | 0.88 | 1 | 0.2295868 |
| Tecpr1 | 0.938816 | 0.566295643 | 0.441 | 0.706 | 1 | 0.0003884 |
| Hmha1 | 0.007232 | 0.57382508 | 0.545 | 0.84 | 1 | 0.000404 |
| Ankrd12 | 0.001274 | 0.429614361 | 0.372 | 0.673 | 1 | 0.000494 |
| Lck | 0.000932 | 0.384058147 | 0.595 | 0.884 | 1 | 0.2133148 |
| Arhgap30 | 0.001641 | 0.324881446 | 0.411 | 0.77 | 1 | 0.0010542 |
| Arid5a | 0.027397 | 0.567071825 | 0.379 | 0.664 | 1 | 0.0014104 |
| Lyst | 0.001944 | 0.579051686 | 0.333 | 0.591 | 1 | 0.03666 |
| Dock10 | 0.003192 | 0.364284764 | 0.446 | 0.821 | 1 | 0.0019762 |
| Ccnl1 | 0.182992 | 0.59672543 | 0.49 | 0.774 | 1 | 0.0023928 |
| Sf3b1 | 0.002465 | 0.43254729 | 0.605 | 0.905 | 1 | 0.2444548 |
| Neat1 | 0.090244 | 0.659344952 | 0.333 | 0.535 | 1 | 0.0031641 |
| H2-Q6 | 0.128767 | 0.517528051 | 0.448 | 0.769 | 1 | 0.0041034 |
| Btg2 | 0.013652 | 0.34559324 | 0.49 | 0.842 | 1 | 0.006572 |
| Rsrp1 | 0.194192 | 0.415046849 | 0.57 | 0.891 | 1 | 0.0072434 |
| Chd7 | 0.008633 | 0.567901861 | 0.533 | 0.801 | 1 | 0.82985 |
| Clk1 | 0.019191 | 0.579837242 | 0.517 | 0.801 | 1 | 0.0646196 |
| Ncor1 | 0.031366 | 0.300063446 | 0.483 | 0.869 | 1 | 0.0262509 |
| Chd2 | 0.052343 | 0.490236019 | 0.393 | 0.673 | 1 | 0.028718 |
| Wnk1 | 0.030817 | 0.360731383 | 0.586 | 0.892 | 1 | 0.8397891 |
| Ankrd44 | 0.814119 | 0.520734475 | 0.457 | 0.765 | 1 | 0.031458 |
| Prrc2c | 0.976678 | 0.402179862 | 0.554 | 0.892 | 1 | 0.0462576 |
| Utrn | 0.228615 | 0.710633844 | 0.439 | 0.664 | 1 | 0.0488748 |
| Nktr | 0.313243 | 0.595963087 | 0.49 | 0.799 | 1 | 0.0680932 |
| Luc7l2 | 0.299566 | 0.421350844 | 0.543 | 0.874 | 1 | 0.0813143 |
| mt-Nd6 | 0.140784 | 0.405479489 | 0.17 | 0.234 | 1 | 0.4863416 |
| Whsc1l1 | 0.31096 | 0.484522093 | 0.474 | 0.821 | 1 | 0.2485264 |
| Zfp292 | 0.366303 | 0.488069922 | 0.462 | 0.793 | 1 | 0.2789839 |
| Mycbp2 | 0.332826 | 0.529751185 | 0.444 | 0.768 | 1 | 0.9721877 |
| Iqgap1 | 0.436654 | 0.29307506 | 0.536 | 0.889 | 1 | 0.4691463 |
| Kmt2a | 0.882749 | 0.657614839 | 0.437 | 0.713 | 1 | 0.8823643 |

| | | | | | | minimum |
|---|---|---|---|---|---|---|
| | Male_avg_logFC | Male_pct.1 | Male_pct.2 | Male_p_val_adj | max_pval | p_p_val |
| mt-Co1 | 1.164624679 | 1 | 1 | 1.16E−230 | 6.95E−142 | 1.48E−234 |
| mt-Co2 | 1.200737284 | 1 | 1 | 2.35E−212 | 7.39E−139 | 3.02E−216 |
| mt-Atp6 | 1.1908109 | 1 | 1 | 4.85E−200 | 1.07E−127 | 6.21E−204 |
| mt-Co3 | 1.21942118 | 1 | 1 | 3.31E−194 | 1.46E−130 | 4.24E−198 |
| Rpl10 | −0.82910948 | 0.738 | 0.978 | 7.56E−189 | 9.41E−138 | 9.70E−193 |
| Rps2 | −0.79443558 | 0.838 | 0.987 | 1.06E−184 | 2.07E−123 | 1.36E−188 |
| Rpl5 | −0.72024468 | 0.803 | 0.98 | 4.51E−177 | 6.19E−117 | 5.78E−181 |
| mt-Nd4 | 1.151337946 | 0.969 | 0.992 | 6.67E−176 | 9.68E−103 | 8.55E−180 |
| Eef1a1 | −0.65995031 | 0.95 | 0.995 | 3.21E−175 | 2.76E−109 | 4.11E−179 |
| mt-Nd1 | 1.102229198 | 0.973 | 0.993 | 2.51E−173 | 1.06E−98 | 3.22E−177 |
| Gnb2l1 | −0.66088837 | 0.832 | 0.982 | 6.72E−173 | 1.72E−108 | 8.61E−177 |
| mt-Cytb | 1.164908289 | 0.973 | 0.989 | 3.25E−172 | 5.00E−98 | 4.16E−176 |
| Rps15a | −0.70516129 | 0.929 | 0.992 | 9.75E−172 | 5.15E−110 | 1.25E−175 |
| Rpl7 | −0.66402718 | 0.783 | 0.98 | 2.64E−165 | 9.19E−116 | 3.38E−169 |
| Btf3 | −0.80915189 | 0.419 | 0.941 | 6.81E−165 | 2.11E−101 | 8.73E−169 |
| Rpl32 | −0.66443545 | 0.909 | 0.989 | 1.14E−164 | 2.43E−93 | 1.46E−168 |
| Rplp0 | −0.65630801 | 0.828 | 0.982 | 1.54E−161 | 5.17E−118 | 1.97E−165 |
| Rps13 | −0.65290986 | 0.892 | 0.986 | 5.40E−160 | 3.84E−103 | 6.92E−164 |
| Tpt1 | −0.66006217 | 0.99 | 0.998 | 3.37E−159 | 5.93E−108 | 4.32E−163 |
| Rps27a | −0.64693985 | 0.873 | 0.989 | 4.73E−159 | 3.65E−116 | 6.06E−163 |
| Gm42418 | 1.570853626 | 0.97 | 0.994 | 8.20E−159 | 3.32E−126 | 1.05E−162 |
| Rps25 | −0.72409148 | 0.661 | 0.962 | 4.73E−158 | 9.86E−113 | 6.06E−162 |
| Rps18 | −0.64319484 | 0.906 | 0.988 | 1.93E−156 | 1.16E−89 | 2.47E−160 |
| Rpl3 | −0.65699755 | 0.832 | 0.981 | 3.06E−156 | 1.13E−100 | 3.92E−160 |
| Rps3a1 | −0.58675152 | 0.896 | 0.988 | 3.29E−156 | 3.00E−114 | 4.22E−160 |
| Rpl9 | −0.64575968 | 0.87 | 0.986 | 1.63E−155 | 4.81E−114 | 2.10E−159 |
| Rpl12 | −0.7232224 | 0.849 | 0.982 | 1.70E−155 | 3.94E−107 | 2.18E−159 |
| Rpl23a | −0.74561506 | 0.623 | 0.959 | 1.98E−154 | 6.86E−119 | 2.53E−158 |
| Rps10 | −0.64828999 | 0.869 | 0.984 | 1.25E−153 | 3.75E−112 | 1.61E−157 |
| Rpl18 | −0.62998856 | 0.803 | 0.987 | 7.76E−153 | 4.96E−105 | 9.95E−157 |
| Rps7 | −0.66009039 | 0.868 | 0.986 | 1.17E−152 | 5.66E−122 | 1.50E−156 |
| Rpl39 | −0.6838299 | 0.829 | 0.982 | 1.84E−150 | 6.11E−126 | 2.36E−154 |
| Rpl30 | −0.62024735 | 0.853 | 0.985 | 3.71E−149 | 1.59E−108 | 4.75E−153 |
| mt-Nd2 | 1.141339482 | 0.974 | 0.993 | 7.95E−149 | 2.29E−95 | 1.02E−152 |
| Rpl23 | −0.5898141 | 0.937 | 0.994 | 1.33E−147 | 2.35E−103 | 1.70E−151 |
| Rps23 | −0.61421422 | 0.879 | 0.986 | 1.33E−147 | 1.52E−88 | 1.70E−151 |
| Rps6 | −0.59963722 | 0.849 | 0.98 | 2.08E−144 | 4.81E−87 | 2.67E−148 |
| Rpl15 | −0.76605226 | 0.449 | 0.936 | 2.42E−144 | 5.68E−119 | 3.10E−148 |
| Malat1 | 0.888909283 | 0.997 | 1 | 4.74E−144 | 3.49E−89 | 6.07E−148 |
| Rps3 | −0.60156103 | 0.88 | 0.985 | 3.37E−143 | 3.73E−109 | 4.32E−147 |
| Rps16 | −0.62578193 | 0.92 | 0.989 | 8.01E−141 | 4.41E−95 | 1.03E−144 |
| Rps24 | −0.65712552 | 0.934 | 0.991 | 1.26E−138 | 3.32E−104 | 1.61E−142 |
| Eef1b2 | −0.65698867 | 0.638 | 0.963 | 1.92E−138 | 7.53E−118 | 2.46E−142 |
| Eif1 | −0.59796703 | 0.714 | 0.975 | 7.36E−138 | 7.21E−92 | 9.44E−142 |
| Rps20 | −0.63501526 | 0.89 | 0.989 | 1.44E−137 | 4.09E−91 | 1.85E−141 |
| Ubb | −0.63325343 | 0.735 | 0.969 | 1.71E−137 | 4.61E−97 | 2.19E−141 |
| Rpl27a | −0.57313188 | 0.892 | 0.992 | 8.99E−137 | 2.14E−84 | 1.15E−140 |
| Rpl34 | −0.58101335 | 0.836 | 0.979 | 4.32E−136 | 6.89E−96 | 5.54E−140 |
| Rps4x | −0.58444933 | 0.858 | 0.983 | 6.29E−135 | 4.33E−81 | 8.06E−139 |
| Ppia | −0.66259875 | 0.715 | 0.974 | 2.98E−134 | 3.50E−127 | 3.82E−138 |
| Rplp2 | −0.56834872 | 0.879 | 0.986 | 3.71E−133 | 3.63E−94 | 4.76E−137 |
| Naca | −0.63901935 | 0.567 | 0.956 | 2.90E−130 | 2.95E−93 | 3.72E−134 |
| mt-Nd5 | 1.14212068 | 0.859 | 0.924 | 2.09E−129 | 1.84E−79 | 2.68E−133 |
| Rpl36a | −0.7064817 | 0.594 | 0.958 | 3.48E−129 | 2.70E−91 | 4.46E−133 |
| Rps9 | −0.5588269 | 0.856 | 0.982 | 1.06E−128 | 2.70E−82 | 1.36E−132 |
| Rps14 | −0.57467568 | 0.893 | 0.984 | 1.49E−128 | 3.29E−84 | 1.91E−132 |
| Rbm3 | −0.69408826 | 0.489 | 0.943 | 3.52E−127 | 8.12E−95 | 4.51E−131 |
| Rps11 | −0.53014386 | 0.883 | 0.983 | 6.09E−127 | 1.03E−69 | 7.81E−131 |
| Rpl11 | −0.50730556 | 0.866 | 0.987 | 8.12E−127 | 1.43E−83 | 1.04E−130 |
| Rpl29 | −0.59379969 | 0.642 | 0.959 | 1.80E−125 | 9.57E−86 | 2.30E−129 |
| Rpl18a | −0.5518132 | 0.896 | 0.987 | 1.61E−124 | 5.20E−72 | 2.06E−128 |
| Tmsb4x | −0.61428435 | 0.991 | 0.999 | 2.90E−124 | 1.28E−93 | 3.72E−128 |
| Pfn1 | −0.70751988 | 0.63 | 0.952 | 3.34E−124 | 7.30E−87 | 4.28E−128 |
| Rps12 | −0.57382613 | 0.897 | 0.984 | 8.03E−124 | 6.10E−70 | 1.03E−127 |
| Rpl35 | −0.64616965 | 0.618 | 0.953 | 8.32E−124 | 8.63E−78 | 1.07E−127 |
| Cox6a1 | −0.54967278 | 0.121 | 0.713 | 5.93E−123 | 7.89E−81 | 7.61E−127 |
| Cox5a | −0.66642286 | 0.229 | 0.832 | 7.35E−123 | 3.22E−93 | 9.42E−127 |
| Erh | −0.59112855 | 0.151 | 0.756 | 9.51E−123 | 1.45E−88 | 1.22E−126 |
| Rps5 | −0.57791001 | 0.85 | 0.98 | 1.13E−121 | 6.57E−87 | 1.45E−125 |
| Rpl37 | −0.56122453 | 0.793 | 0.978 | 1.27E−121 | 7.41E−73 | 1.63E−125 |
| Rps26 | −0.576184 | 0.742 | 0.972 | 3.90E−121 | 2.30E−74 | 4.99E−125 |
| Snrpf | −0.57023961 | 0.168 | 0.778 | 1.91E−120 | 8.80E−59 | 2.45E−124 |
| Rps29 | −0.56801227 | 0.863 | 0.98 | 3.74E−120 | 2.10E−70 | 4.79E−124 |
| Rps17 | −0.69891583 | 0.517 | 0.94 | 8.94E−120 | 1.80E−85 | 1.15E−123 |
| Atp5g1 | −0.56435582 | 0.12 | 0.696 | 3.16E−119 | 9.16E−79 | 4.05E−123 |
| Gabarap | −0.53174764 | 0.192 | 0.81 | 4.94E−119 | 1.01E−55 | 6.33E−123 |
| Rpl13 | −0.50779085 | 0.956 | 0.995 | 3.42E−118 | 3.82E−75 | 4.38E−122 |
| Ndufa4 | −0.53305186 | 0.185 | 0.802 | 5.14E−118 | 1.58E−85 | 6.59E−122 |
| Rpl19 | −0.51188281 | 0.913 | 0.987 | 4.71E−117 | 1.19E−63 | 6.04E−121 |
| Sumo2 | −0.59970985 | 0.252 | 0.874 | 9.14E−117 | 2.24E−76 | 1.17E−120 |
| Eif3i | −0.52315676 | 0.168 | 0.776 | 9.03E−116 | 7.00E−69 | 1.16E−119 |
| Rpl17 | −0.51899153 | 0.925 | 0.987 | 2.31E−115 | 1.40E−49 | 2.96E−119 |
| Prdx1 | −0.49341045 | 0.14 | 0.722 | 3.11E−115 | 3.35E−83 | 3.98E−119 |
| Tbca | −0.39900807 | 0.107 | 0.675 | 5.71E−115 | 1.29E−85 | 7.32E−119 |
| Psma2 | −0.48975344 | 0.162 | 0.76 | 1.56E−113 | 1.44E−83 | 2.01E−117 |
| H3f3a | −0.60236979 | 0.457 | 0.943 | 7.76E−113 | 4.16E−90 | 9.95E−117 |
| Slc25a4 | −0.52629653 | 0.14 | 0.712 | 1.18E−112 | 2.30E−64 | 1.52E−116 |
| Sf3b6 | −0.37685899 | 0.115 | 0.679 | 5.93E−112 | 1.18E−88 | 7.60E−116 |
| Psme2 | −0.52903934 | 0.177 | 0.766 | 8.38E−112 | 1.68E−58 | 1.07E−115 |
| Atp5c1 | −0.54691948 | 0.195 | 0.797 | 1.08E−111 | 1.94E−67 | 1.38E−115 |
| Rplp1 | −0.54041795 | 0.919 | 0.985 | 1.18E−111 | 3.49E−76 | 1.51E−115 |
| Cox7b | −0.50173847 | 0.162 | 0.753 | 1.21E−110 | 4.58E−89 | 1.55E−114 |
| Rpl14 | −0.54046559 | 0.721 | 0.969 | 1.56E−110 | 3.20E−70 | 2.00E−114 |
| Npm1 | −0.68630682 | 0.57 | 0.951 | 2.01E−110 | 9.19E−88 | 2.57E−114 |
| Rps21 | −0.5464437 | 0.826 | 0.984 | 2.24E−110 | 2.55E−72 | 2.87E−114 |
| mt-Nd3 | 1.078336131 | 0.86 | 0.927 | 2.80E−110 | 7.70E−67 | 3.58E−114 |
| Tagln2 | −0.5382618 | 0.181 | 0.765 | 2.88E−110 | 1.23E−85 | 3.69E−114 |
| Rpl22 | −0.55422685 | 0.648 | 0.959 | 3.07E−110 | 7.36E−78 | 3.93E−114 |
| Slc25a5 | −0.51227085 | 0.308 | 0.846 | 6.21E−83 | 3.98E−87 | 2.92E−113 |
| Edf1 | −0.45232029 | 0.147 | 0.725 | 2.74E−109 | 9.14E−46 | 3.52E−113 |
| Ran | −0.63721174 | 0.217 | 0.79 | 2.77E−109 | 7.66E−85 | 3.55E−113 |
| Eif3e | −0.57773602 | 0.274 | 0.874 | 4.06E−109 | 4.04E−64 | 5.20E−113 |
| Anp32a | −0.43754397 | 0.131 | 0.701 | 8.12E−109 | 4.02E−93 | 1.04E−112 |
| Eif5a | −0.71060339 | 0.322 | 0.881 | 1.96E−108 | 1.79E−70 | 2.51E−112 |
| Ndufa7 | −0.37189335 | 0.124 | 0.686 | 2.02E−108 | 9.91E−71 | 2.58E−112 |
| Rpl27 | −0.53660901 | 0.627 | 0.955 | 6.21E−108 | 2.73E−101 | 7.97E−112 |
| Cox5b | −0.53623132 | 0.194 | 0.776 | 3.76E−107 | 5.01E−66 | 4.82E−111 |
| Txn1 | −0.62048196 | 0.252 | 0.834 | 6.46E−107 | 1.68E−92 | 8.28E−111 |
| Uqcrb | −0.37791843 | 0.105 | 0.65 | 1.03E−106 | 5.93E−83 | 1.32E−110 |
| Snrpd1 | −0.44946024 | 0.077 | 0.591 | 1.18E−106 | 6.09E−88 | 1.51E−110 |
| Rpl22l1 | −0.60431744 | 0.453 | 0.928 | 1.48E−106 | 6.98E−81 | 1.90E−110 |
| Cox8a | −0.60611797 | 0.403 | 0.928 | 1.82E−106 | 1.64E−98 | 2.34E−110 |
| Rpl8 | −0.45723874 | 0.88 | 0.986 | 2.05E−106 | 1.96E−47 | 2.63E−110 |
| Ndufa6 | −0.41759711 | 0.178 | 0.77 | 2.63E−106 | 2.25E−72 | 3.37E−110 |
| Cox7a2 | −0.48419559 | 0.204 | 0.798 | 3.08E−106 | 1.99E−65 | 3.95E−110 |
| Arhgdib | −0.61929229 | 0.35 | 0.889 | 7.62E−106 | 5.54E−70 | 9.77E−110 |
| S100a11 | −0.59639929 | 0.197 | 0.762 | 1.47E−105 | 1.59E−65 | 1.88E−109 |
| Ost4 | −0.39478855 | 0.11 | 0.651 | 1.86E−105 | 1.55E−88 | 2.38E−109 |
| Usmg5 | −0.44025132 | 0.06 | 0.56 | 2.26E−105 | 2.79E−85 | 2.90E−109 |
| Psmb3 | −0.46600555 | 0.164 | 0.742 | 3.42E−105 | 2.43E−79 | 4.38E−109 |
| Snrpg | −0.47846659 | 0.199 | 0.799 | 6.23E−105 | 2.37E−89 | 7.98E−109 |
| Ndufb8 | −0.44103487 | 0.104 | 0.64 | 1.40E−104 | 7.70E−69 | 1.79E−108 |
| Myl12a | −0.6008526 | 0.256 | 0.827 | 1.82E−104 | 3.99E−88 | 2.33E−108 |
| Ndufb9 | −0.42271274 | 0.128 | 0.68 | 3.62E−104 | 1.57E−74 | 4.64E−108 |
| Magoh | −0.39998354 | 0.08 | 0.594 | 3.88E−104 | 9.36E−89 | 4.97E−108 |
| 2700060E02Rik | −0.41465243 | 0.158 | 0.73 | 1.32E−103 | 1.86E−75 | 1.69E−107 |
| Pomp | −0.45458121 | 0.189 | 0.774 | 3.35E−103 | 7.34E−59 | 4.30E−107 |
| Atp5l | −0.58736307 | 0.268 | 0.868 | 4.84E−103 | 5.28E−78 | 6.20E−107 |
| Psmb2 | −0.41184014 | 0.1 | 0.625 | 1.18E−102 | 1.60E−85 | 1.51E−106 |
| Ndufb11 | −0.43699564 | 0.181 | 0.767 | 1.19E−102 | 5.71E−80 | 1.53E−106 |
| Cfl1 | −0.62175116 | 0.464 | 0.92 | 1.31E−102 | 3.32E−81 | 1.68E−106 |
| Tomm22 | −0.37698003 | 0.132 | 0.684 | 2.39E−102 | 1.21E−86 | 3.07E−106 |
| Oaz1 | −0.55222602 | 0.526 | 0.947 | 3.12E−102 | 2.15E−68 | 4.00E−106 |
| Myl12b | −0.50666903 | 0.245 | 0.847 | 3.84E−102 | 1.59E−67 | 4.92E−106 |
| Atp50 | −0.4359777 | 0.154 | 0.715 | 5.42E−102 | 1.41E−58 | 6.95E−106 |
| Snrpd2 | −0.39867711 | 0.154 | 0.713 | 9.98E−102 | 5.30E−59 | 1.28E−105 |
| Cct8 | −0.4029611 | 0.111 | 0.641 | 1.20E−101 | 4.11E−79 | 1.53E−105 |
| Rpl10a | −0.48655726 | 0.823 | 0.976 | 1.81E−101 | 1.24E−48 | 2.32E−105 |
| Brk1 | −0.30410496 | 0.128 | 0.679 | 2.58E−101 | 8.62E−78 | 3.31E−105 |
| Snx3 | −0.37404069 | 0.094 | 0.608 | 4.21E−101 | 1.10E−71 | 5.39E−105 |
| Sin3b | −0.39032655 | 0.09 | 0.6 | 7.87E−101 | 4.32E−72 | 1.01E−104 |
| Tspo | −0.42091969 | 0.238 | 0.837 | 1.25E−100 | 4.17E−67 | 1.61E−104 |
| Nhp2l1 | −0.40251448 | 0.121 | 0.657 | 1.29E−100 | 3.04E−83 | 1.65E−104 |
| Lamtor5 | −0.367174 | 0.066 | 0.557 | 2.30E−100 | 2.46E−79 | 2.95E−104 |
| Sri | −0.36300895 | 0.177 | 0.756 | 1.09E−99 | 1.47E−74 | 1.40E−103 |
| Phb2 | −0.39823651 | 0.12 | 0.651 | 1.31E−99 | 4.66E−62 | 1.67E−103 |
| Nme1 | −0.47957597 | 0.125 | 0.658 | 1.45E−99 | 3.80E−70 | 1.86E−103 |
| Cycs | −0.4940587 | 0.144 | 0.679 | 2.07E−99 | 5.82E−81 | 2.65E−103 |
| Atp5j | −0.51058751 | 0.209 | 0.786 | 2.44E−99 | 4.63E−62 | 3.12E−103 |
| Gabarapl2 | −0.35898191 | 0.127 | 0.666 | 2.49E−99 | 5.08E−83 | 3.19E−103 |
| Psma7 | −0.42765725 | 0.199 | 0.776 | 3.16E−99 | 7.54E−68 | 4.06E−103 |
| Taldo1 | −0.35107085 | 0.09 | 0.599 | 3.22E−99 | 2.33E−84 | 4.13E−103 |
| Snrpe | −0.53551513 | 0.274 | 0.852 | 4.47E−99 | 4.64E−98 | 5.73E−103 |
| Ndufb5 | −0.35753338 | 0.154 | 0.716 | 4.92E−99 | 7.53E−85 | 6.31E−103 |
| Psmb5 | −0.40534602 | 0.115 | 0.643 | 9.62E−99 | 6.88E−75 | 1.23E−102 |
| Ube2l3 | −0.39439825 | 0.142 | 0.691 | 1.32E−98 | 1.90E−79 | 1.69E−102 |
| Psma5 | −0.35703685 | 0.071 | 0.563 | 1.38E−98 | 5.14E−74 | 1.77E−102 |
| Rps28 | −0.56242279 | 0.558 | 0.944 | 1.44E−98 | 2.59E−76 | 1.84E−102 |
| Mpc1 | −0.44900061 | 0.043 | 0.505 | 4.07E−98 | 1.45E−79 | 5.22E−102 |
| Spcs1 | −0.347238 | 0.121 | 0.648 | 8.63E−98 | 3.28E−92 | 1.11E−101 |
| Atp5f1 | −0.49010631 | 0.224 | 0.808 | 1.02E−97 | 1.16E−80 | 1.31E−101 |
| Tceb1 | −0.34977076 | 0.097 | 0.611 | 1.35E−97 | 2.83E−73 | 1.73E−101 |
| Psmb1 | −0.51738986 | 0.278 | 0.869 | 2.01E−97 | 1.89E−63 | 2.58E−101 |
| Rps27l | −0.4468847 | 0.095 | 0.599 | 2.58E−97 | 2.15E−77 | 3.31E−101 |
| Atp6v1f | −0.38120182 | 0.177 | 0.752 | 3.65E−97 | 7.94E−72 | 4.68E−101 |
| Mrpl20 | −0.38761712 | 0.087 | 0.586 | 5.31E−97 | 3.20E−91 | 6.81E−101 |
| Rbx1 | −0.40423704 | 0.171 | 0.729 | 8.25E−97 | 1.17E−72 | 1.06E−100 |
| Lsm5 | −0.42641677 | 0.061 | 0.537 | 8.47E−97 | 1.61E−77 | 1.09E−100 |
| Ssr4 | −0.42415772 | 0.191 | 0.77 | 1.17E−96 | 1.04E−87 | 1.50E−100 |
| Mrps14 | −0.37235696 | 0.077 | 0.568 | 1.58E−96 | 1.24E−79 | 2.03E−100 |
| Sec61b | −0.48500584 | 0.264 | 0.863 | 2.12E−96 | 1.26E−72 | 2.72E−100 |
| Cox6c | −0.51986029 | 0.275 | 0.855 | 2.61E−96 | 8.18E−81 | 3.34E−100 |
| Mettl23 | −0.30736364 | 0.142 | 0.686 | 3.79E−96 | 4.84E−69 | 4.86E−100 |
| Ywhaq | −0.32320381 | 0.115 | 0.639 | 3.94E−96 | 1.94E−70 | 5.05E−100 |
| Rpl24 | −0.45792443 | 0.802 | 0.977 | 5.17E−96 | 6.31E−47 | 6.62E−100 |
| Chchd2 | −0.53628449 | 0.358 | 0.907 | 6.05E−96 | 3.62E−61 | 7.76E−100 |
| Rps8 | −0.4438096 | 0.913 | 0.991 | 6.12E−96 | 9.18E−45 | 7.85E−100 |
| Gng5 | −0.42406407 | 0.244 | 0.834 | 6.87E−96 | 1.12E−76 | 8.81E−100 |
| Snrpb | −0.40103543 | 0.182 | 0.75 | 7.47E−96 | 6.64E−65 | 9.57E−100 |
| Nedd8 | −0.40486769 | 0.178 | 0.75 | 9.08E−96 | 3.91E−68 | 1.16E−99 |
| Pdcd6 | −0.29671913 | 0.093 | 0.594 | 1.04E−95 | 1.24E−71 | 1.34E−99 |
| Esd | −0.34488236 | 0.111 | 0.628 | 3.96E−95 | 5.46E−82 | 5.08E−99 |
| Arpc3 | −0.54421076 | 0.323 | 0.896 | 4.14E−95 | 7.02E−57 | 5.31E−99 |
| Gm10073 | −0.37124036 | 0.113 | 0.627 | 8.28E−95 | 2.07E−91 | 1.06E−98 |
| Aprt | −0.39947318 | 0.101 | 0.606 | 8.65E−95 | 2.42E−79 | 1.11E−98 |
| Rpl21 | −0.45681367 | 0.85 | 0.979 | 1.01E−94 | 3.82E−46 | 1.30E−98 |
| Minos1 | −0.35768616 | 0.142 | 0.681 | 1.33E−94 | 2.13E−64 | 1.70E−98 |
| Srp14 | −0.38290478 | 0.164 | 0.719 | 2.94E−94 | 2.88E−74 | 3.77E−98 |
| Nop10 | −0.38133269 | 0.1 | 0.599 | 3.25E−94 | 3.86E−80 | 4.16E−98 |
| 2410015M20Rik | −0.29801941 | 0.151 | 0.699 | 4.54E−94 | 3.87E−62 | 5.82E−98 |
| Npm3 | −0.3902636 | 0.075 | 0.553 | 4.85E−94 | 3.81E−68 | 6.22E−98 |
| Ndufab1 | −0.37905565 | 0.063 | 0.532 | 7.69E−94 | 2.65E−64 | 9.86E−98 |
| Pebp1 | −0.33742633 | 0.13 | 0.657 | 3.33E−93 | 6.02E−71 | 4.26E−97 |
| Rpl23a-ps3 | −0.35061127 | 0.07 | 0.54 | 3.46E−93 | 1.92E−74 | 4.44E−97 |
| Cdk2ap2 | −0.27754352 | 0.127 | 0.648 | 3.99E−93 | 6.10E−69 | 5.11E−97 |
| Cct2 | −0.41251245 | 0.192 | 0.76 | 7.26E−93 | 5.30E−68 | 9.30E−97 |
| Mif | −0.615429 | 0.322 | 0.841 | 7.67E−93 | 3.11E−64 | 9.83E−97 |
| Manf | −0.43318044 | 0.104 | 0.603 | 1.24E−92 | 8.51E−79 | 1.60E−96 |
| 15-Sep | −0.32959164 | 0.141 | 0.672 | 2.18E−92 | 1.96E−95 | 2.80E−96 |
| Snrpd3 | −0.36591764 | 0.08 | 0.557 | 2.18E−92 | 2.81E−69 | 2.80E−96 |
| Atp6v0e | −0.30230977 | 0.177 | 0.736 | 2.21E−92 | 1.00E−68 | 2.83E−96 |
| Hprt | −0.37250049 | 0.091 | 0.58 | 2.35E−92 | 1.99E−81 | 3.01E−96 |
| 2010107E04Rik | −0.36871861 | 0.184 | 0.743 | 2.35E−92 | 1.71E−55 | 3.01E−96 |
| Bax | −0.32923564 | 0.125 | 0.639 | 2.93E−92 | 6.30E−73 | 3.76E−96 |
| Tmem258 | −0.3197093 | 0.117 | 0.631 | 3.97E−92 | 8.08E−75 | 5.09E−96 |
| Psmb6 | −0.30852057 | 0.107 | 0.611 | 9.41E−92 | 9.02E−62 | 1.21E−95 |
| Eif3m | −0.45876977 | 0.238 | 0.824 | 1.32E−91 | 3.61E−76 | 1.69E−95 |
| Srgn | −0.64676397 | 0.521 | 0.968 | 1.80E−91 | 1.27E−92 | 2.31E−95 |
| Banf1 | −0.39995217 | 0.07 | 0.535 | 3.11E−91 | 7.88E−70 | 3.99E−95 |
| Psmb9 | −0.38209698 | 0.162 | 0.701 | 5.20E−91 | 3.91E−58 | 6.66E−95 |
| Prdx5 | −0.35912421 | 0.123 | 0.635 | 5.96E−91 | 2.00E−69 | 7.64E−95 |
| Sap18 | −0.25385476 | 0.094 | 0.585 | 8.43E−91 | 4.54E−86 | 1.08E−94 |
| Ube2n | −0.31769321 | 0.088 | 0.571 | 1.18E−90 | 1.39E−74 | 1.52E−94 |
| Lsm4 | −0.33506051 | 0.152 | 0.685 | 2.08E−90 | 2.86E−69 | 2.67E−94 |
| Ndufa8 | −0.29383731 | 0.083 | 0.561 | 2.64E−90 | 6.28E−88 | 3.38E−94 |
| Mdh1 | −0.37551829 | 0.118 | 0.621 | 3.00E−90 | 1.01E−59 | 3.84E−94 |
| Swi5 | −0.31236425 | 0.108 | 0.608 | 3.07E−90 | 3.51E−78 | 3.93E−94 |
| Uqcrfs1 | −0.26261501 | 0.138 | 0.666 | 3.08E−90 | 1.30E−73 | 3.95E−94 |
| Vdac2 | −0.32822903 | 0.154 | 0.689 | 4.22E−90 | 1.67E−68 | 5.41E−94 |
| Mrpl33 | −0.37450609 | 0.164 | 0.703 | 5.39E−90 | 3.63E−62 | 6.90E−94 |
| Psmd8 | −0.31213705 | 0.132 | 0.65 | 6.34E−90 | 9.35E−62 | 8.13E−94 |
| Ptma | −0.57759503 | 0.779 | 0.978 | 9.91E−90 | 1.49E−64 | 1.27E−93 |
| Mrfap1 | −0.29153755 | 0.128 | 0.641 | 1.17E−89 | 2.43E−72 | 1.50E−93 |
| Fth1 | −0.50037985 | 0.483 | 0.957 | 1.44E−89 | 1.48E−70 | 1.84E−93 |
| S100a10 | −0.63671752 | 0.417 | 0.897 | 1.46E−89 | 1.41E−66 | 1.87E−93 |
| Eif3k | −0.45314968 | 0.272 | 0.865 | 2.51E−89 | 6.42E−52 | 3.22E−93 |
| Psma1 | −0.29768601 | 0.117 | 0.623 | 3.29E−89 | 5.50E−69 | 4.22E−93 |
| Txnl1 | −0.31232853 | 0.087 | 0.562 | 3.41E−89 | 9.50E−84 | 4.37E−93 |
| Tomm7 | −0.36095823 | 0.182 | 0.737 | 4.42E−89 | 3.79E−74 | 5.66E−93 |
| Rac1 | −0.37047975 | 0.208 | 0.783 | 4.64E−89 | 2.07E−71 | 5.95E−93 |
| Fkbp3 | −0.32982036 | 0.107 | 0.601 | 4.76E−89 | 2.62E−67 | 6.11E−93 |
| Erp44 | −0.3596858 | 0.071 | 0.532 | 8.59E−89 | 1.66E−69 | 1.10E−92 |
| Cacybp | −0.27572204 | 0.094 | 0.576 | 1.03E−88 | 9.15E−76 | 1.32E−92 |
| Pdcd5 | −0.27014905 | 0.101 | 0.59 | 1.18E−88 | 1.82E−73 | 1.51E−92 |
| Dnajc15 | −0.39258844 | 0.225 | 0.787 | 1.87E−88 | 2.19E−67 | 2.40E−92 |
| Uqcrc2 | −0.29525814 | 0.105 | 0.594 | 2.55E−88 | 7.53E−84 | 3.27E−92 |
| Smdt1 | −0.30354037 | 0.142 | 0.658 | 3.25E−88 | 2.35E−59 | 4.17E−92 |
| Rpl6 | −0.43639674 | 0.803 | 0.973 | 3.25E−88 | 1.11E−41 | 4.17E−92 |
| Mdh2 | −0.32908556 | 0.137 | 0.655 | 3.44E−88 | 4.32E−82 | 4.41E−92 |
| Hint1 | −0.50479955 | 0.271 | 0.839 | 3.46E−88 | 1.20E−77 | 4.43E−92 |
| Cap1 | −0.30468009 | 0.145 | 0.663 | 9.39E−88 | 1.21E−57 | 1.20E−91 |
| Sh2d1a | −0.36123622 | 0.11 | 0.598 | 1.30E−87 | 1.06E−69 | 1.66E−91 |
| 1110008F13Rik | −0.28416243 | 0.09 | 0.563 | 2.69E−87 | 4.13E−65 | 3.45E−91 |
| Rpl4 | −0.44631736 | 0.726 | 0.964 | 2.95E−87 | 1.77E−44 | 3.78E−91 |
| Tmem14c | −0.26894357 | 0.124 | 0.626 | 5.64E−87 | 1.65E−83 | 7.24E−91 |
| Psenen | −0.25197137 | 0.167 | 0.708 | 5.87E−87 | 3.40E−76 | 7.52E−91 |
| Ndufv2 | −0.31261458 | 0.073 | 0.53 | 5.91E−87 | 7.70E−72 | 7.58E−91 |
| Rps19 | −0.48077123 | 0.865 | 0.982 | 6.24E−87 | 1.68E−52 | 8.00E−91 |
| Dap | −0.36597072 | 0.093 | 0.564 | 7.27E−87 | 9.99E−62 | 9.33E−91 |
| Eif6 | −0.25804899 | 0.078 | 0.544 | 8.32E−87 | 2.30E−80 | 1.07E−90 |
| Bzw1 | −0.30100365 | 0.165 | 0.702 | 8.32E−87 | 2.20E−58 | 1.07E−90 |
| Tomm5 | −0.31690775 | 0.11 | 0.594 | 1.29E−86 | 5.08E−71 | 1.65E−90 |
| Polr1d | −0.40437435 | 0.245 | 0.816 | 2.40E−86 | 3.70E−67 | 3.08E−90 |
| Eef1d | −0.45623433 | 0.276 | 0.857 | 2.50E−86 | 1.59E−46 | 3.20E−90 |
| Rpl35a | −0.4122645 | 0.852 | 0.977 | 3.43E−86 | 1.67E−40 | 4.40E−90 |
| Rpl7a | −0.44513963 | 0.704 | 0.966 | 4.90E−86 | 2.20E−34 | 6.28E−90 |
| Pgam1 | −0.42954474 | 0.168 | 0.688 | 5.96E−86 | 6.28E−72 | 7.64E−90 |
| Krtcap2 | −0.38224715 | 0.222 | 0.78 | 6.16E−86 | 4.36E−73 | 7.90E−90 |
| Rpl28 | −0.4318373 | 0.848 | 0.98 | 1.17E−85 | 7.79E−46 | 1.50E−89 |
| ler3ip1 | −0.26641536 | 0.084 | 0.549 | 1.20E−85 | 1.08E−74 | 1.54E−89 |
| Arf1 | −0.31986323 | 0.215 | 0.76 | 3.22E−79 | 2.06E−83 | 1.89E−89 |
| Shfm1 | −0.43470408 | 0.245 | 0.814 | 3.40E−85 | 1.54E−51 | 4.35E−89 |
| Eif1ax | −0.33703104 | 0.083 | 0.542 | 6.34E−85 | 3.71E−77 | 8.13E−89 |
| Atp5g3 | −0.38522963 | 0.188 | 0.721 | 6.65E−85 | 3.14E−73 | 8.53E−89 |
| Vapa | −0.25259536 | 0.08 | 0.538 | 7.75E−85 | 2.10E−79 | 9.93E−89 |
| Raly | −0.27770043 | 0.147 | 0.666 | 8.24E−85 | 1.58E−63 | 1.06E−88 |
| Emp3 | −0.38339601 | 0.165 | 0.679 | 1.52E−84 | 2.99E−71 | 1.95E−88 |
| Rpl38 | −0.43222044 | 0.829 | 0.975 | 1.65E−84 | 1.46E−44 | 2.12E−88 |
| Selk | −0.26212179 | 0.201 | 0.759 | 2.28E−84 | 1.25E−62 | 2.92E−88 |
| Ube2s | −0.37204719 | 0.165 | 0.679 | 2.32E−84 | 9.75E−54 | 2.97E−88 |
| Eif2s2 | −0.41824368 | 0.212 | 0.756 | 2.47E−84 | 6.65E−56 | 3.16E−88 |
| Calm1 | −0.51581055 | 0.447 | 0.921 | 2.67E−84 | 1.04E−66 | 3.42E−88 |
| Vps29 | −0.26551914 | 0.075 | 0.528 | 3.31E−84 | 1.67E−66 | 4.24E−88 |
| Tomm20 | −0.47365324 | 0.309 | 0.887 | 4.10E−84 | 5.69E−69 | 5.26E−88 |
| Cwc15 | −0.28662902 | 0.131 | 0.627 | 5.08E−84 | 2.26E−63 | 6.52E−88 |
| Fau | −0.3996734 | 0.93 | 0.992 | 6.51E−84 | 1.06E−42 | 8.34E−88 |
| Csnk2b | 0.29032648 | 0.145 | 0.655 | 7.45E−84 | 2.66E−67 | 9.55E−88 |
| Fundc2 | −0.28149999 | 0.094 | 0.561 | 9.31E−84 | 1.99E−83 | 1.19E−87 |
| Mrpl23 | −0.33192891 | 0.06 | 0.496 | 1.04E−83 | 5.30E−59 | 1.34E−87 |
| Tma7 | −0.45421226 | 0.276 | 0.844 | 1.11E−83 | 1.13E−39 | 1.42E−87 |
| Uqcrh | −0.49006802 | 0.427 | 0.929 | 2.27E−83 | 2.52E−57 | 2.91E−87 |
| Cotl1 | −0.45302983 | 0.269 | 0.819 | 2.65E−83 | 4.33E−55 | 3.40E−87 |
| Hnrnpa1 | −0.49218642 | 0.241 | 0.774 | 3.30E−83 | 5.41E−67 | 4.24E−87 |
| Gmfg | −0.4510332 | 0.265 | 0.828 | 4.54E−83 | 1.29E−48 | 5.82E−87 |
| Ndufa12 | −0.31289591 | 0.063 | 0.5 | 7.41E−83 | 5.26E−69 | 9.50E−87 |
| Rps27 | −0.49712253 | 0.85 | 0.977 | 1.13E−82 | 1.19E−55 | 1.45E−86 |
| Dbi | −0.35771094 | 0.093 | 0.552 | 1.18E−82 | 2.34E−71 | 1.51E−86 |
| H2afz | −0.44262106 | 0.407 | 0.89 | 2.05E−59 | 1.32E−63 | 2.23E−86 |
| Atp5g2 | −0.46933861 | 0.349 | 0.888 | 2.33E−82 | 3.40E−75 | 2.99E−86 |
| Psmb4 | −0.33494349 | 0.17 | 0.683 | 2.48E−82 | 2.90E−59 | 3.18E−86 |
| Uqcrq | −0.33748568 | 0.17 | 0.686 | 3.30E−82 | 1.69E−46 | 4.23E−86 |
| Eno1 | −0.56697568 | 0.339 | 0.842 | 4.53E−82 | 4.17E−61 | 5.81E−86 |
| Prdx2 | −0.30088069 | 0.127 | 0.617 | 5.19E−82 | 4.41E−61 | 6.66E−86 |
| Hspe1 | −0.480148 | 0.318 | 0.856 | 5.88E−82 | 7.85E−64 | 7.54E−86 |
| Ube2m | −0.30523976 | 0.091 | 0.551 | 9.61E−82 | 1.56E−60 | 1.23E−85 |
| Sepw1 | −0.31055601 | 0.199 | 0.742 | 1.42E−81 | 6.48E−58 | 1.82E−85 |
| Fam96a | −0.28397855 | 0.061 | 0.492 | 2.62E−81 | 6.28E−78 | 3.36E−85 |
| Hnrnpc | −0.28875357 | 0.174 | 0.696 | 2.82E−81 | 9.29E−69 | 3.62E−85 |
| Ppp1r14b | −0.34933992 | 0.08 | 0.526 | 2.96E−81 | 4.05E−64 | 3.80E−85 |
| Sumo1 | −0.26341551 | 0.172 | 0.704 | 3.33E−81 | 1.75E−83 | 4.27E−85 |
| Mrps16 | −0.29739667 | 0.142 | 0.641 | 3.48E−81 | 2.02E−65 | 4.47E−85 |
| U2af1 | −0.29071692 | 0.101 | 0.568 | 5.16E−81 | 4.42E−69 | 6.61E−85 |
| Grpel1 | −0.2798996 | 0.046 | 0.461 | 7.74E−81 | 1.42E−72 | 9.92E−85 |
| Nudt21 | −0.25934697 | 0.11 | 0.582 | 7.98E−81 | 9.60E−65 | 1.02E−84 |
| Ubl5 | −0.46162238 | 0.289 | 0.835 | 1.42E−80 | 1.05E−65 | 1.82E−84 |
| Sec61g | −0.40993204 | 0.279 | 0.833 | 1.52E−80 | 6.53E−59 | 1.95E−84 |
| Ywhah | −0.32779657 | 0.11 | 0.579 | 2.00E−80 | 4.18E−58 | 2.57E−84 |
| Arf4 | −0.25057438 | 0.107 | 0.576 | 2.21E−80 | 1.38E−73 | 2.83E−84 |
| Fam162a | −0.41096313 | 0.07 | 0.5 | 2.26E−80 | 2.32E−61 | 2.89E−84 |
| Ube2a | −0.3045887 | 0.061 | 0.484 | 1.72E−79 | 1.10E−83 | 3.44E−84 |
| Tmsb10 | −0.47674633 | 0.764 | 0.969 | 3.11E−80 | 1.42E−47 | 3.98E−84 |
| Ywhae | −0.2985786 | 0.158 | 0.666 | 3.32E−80 | 1.61E−73 | 4.25E−84 |
| Epsti1 | −0.34752569 | 0.217 | 0.757 | 3.79E−80 | 2.22E−49 | 4.86E−84 |
| Ubald2 | −0.31806792 | 0.128 | 0.608 | 4.54E−80 | 5.31E−58 | 5.82E−84 |
| Psme1 | −0.42095272 | 0.292 | 0.854 | 6.22E−80 | 4.41E−52 | 7.98E−84 |
| Eef1g | −0.4957789 | 0.446 | 0.924 | 7.27E−80 | 2.78E−56 | 9.32E−84 |
| Rnf7 | −0.25219773 | 0.077 | 0.517 | 7.64E−80 | 2.80E−65 | 9.80E−84 |
| Ranbp1 | −0.41469911 | 0.11 | 0.568 | 1.58E−79 | 1.03E−77 | 2.03E−83 |
| Ndufa1 | −0.2516656 | 0.135 | 0.626 | 2.70E−79 | 5.46E−61 | 3.46E−83 |
| Atp5e | −0.4775333 | 0.385 | 0.896 | 2.73E−79 | 5.95E−33 | 3.49E−83 |
| Ndufs5 | −0.27991605 | 0.068 | 0.498 | 3.84E−79 | 7.80E−80 | 4.92E−83 |
| Atp5k | −0.28088525 | 0.144 | 0.636 | 9.27E−79 | 6.58E−53 | 1.19E−82 |
| Clic1 | −0.45919428 | 0.37 | 0.914 | 1.07E−78 | 9.38E−80 | 1.37E−82 |
| Pycard | 0.31289666 | 0.128 | 0.602 | 1.35E−78 | 1.02E−67 | 1.73E−82 |
| Dad1 | −0.34092993 | 0.262 | 0.829 | 2.22E−78 | 1.72E−55 | 2.84E−82 |
| Cmpk1 | −0.26470852 | 0.091 | 0.535 | 3.35E−78 | 1.50E−69 | 4.29E−82 |
| Commd3 | −0.2792083 | 0.056 | 0.469 | 3.60E−78 | 9.07E−71 | 4.62E−82 |
| Mien1 | −0.26147905 | 0.104 | 0.559 | 4.44E−78 | 2.22E−81 | 5.69E−82 |
| Hnrnpab | −0.32315378 | 0.168 | 0.666 | 5.33E−78 | 3.11E−66 | 6.83E−82 |
| Gm10076 | −0.31533427 | 0.221 | 0.764 | 5.84E−78 | 5.32E−47 | 7.48E−82 |
| Ostc | −0.28779053 | 0.15 | 0.645 | 6.75E−78 | 4.09E−75 | 8.66E−82 |
| Akr1a1 | −0.25220338 | 0.145 | 0.637 | 6.76E−78 | 1.15E−75 | 8.67E−82 |
| Polr2g | −0.26325047 | 0.066 | 0.489 | 8.25E−78 | 1.26E−74 | 1.06E−81 |
| Crip1 | −0.57799408 | 0.303 | 0.821 | 1.06E−77 | 3.85E−37 | 1.36E−81 |
| Rap1a | −0.31946868 | 0.218 | 0.749 | 5.18E−74 | 3.32E−78 | 1.93E−81 |
| Nol7 | −0.28662093 | 0.202 | 0.735 | 2.48E−77 | 4.65E−50 | 3.18E−81 |
| Ndfip1 | −0.31131286 | 0.131 | 0.599 | 3.51E−77 | 1.23E−68 | 4.50E−81 |
| Cct5 | −0.29247405 | 0.172 | 0.682 | 3.60E−77 | 3.58E−52 | 4.61E−81 |
| Rpl26 | −0.3927293 | 0.869 | 0.98 | 4.52E−77 | 2.26E−33 | 5.79E−81 |
| Glrx5 | −0.29610378 | 0.046 | 0.446 | 4.83E−77 | 4.19E−64 | 6.19E−81 |
| 2700094K13Rik | −0.33999004 | 0.081 | 0.512 | 8.05E−77 | 5.52E−51 | 1.03E−80 |
| Actg1 | −0.53719954 | 0.852 | 0.992 | 1.40E−76 | 1.37E−72 | 1.80E−80 |
| Nhp2 | −0.38305373 | 0.056 | 0.462 | 1.60E−76 | 7.63E−54 | 2.05E−80 |
| Anp32e | −0.32270045 | 0.088 | 0.523 | 1.92E−76 | 1.39E−51 | 2.46E−80 |
| Lsm6 | −0.2529147 | 0.081 | 0.513 | 4.80E−76 | 3.36E−62 | 6.16E−80 |
| Abracl | −0.40023701 | 0.268 | 0.809 | 7.01E−76 | 1.97E−63 | 8.99E−80 |
| Arpc2 | −0.45709054 | 0.476 | 0.927 | 8.19E−76 | 5.85E−61 | 1.05E−79 |
| Polr2k | −0.26049475 | 0.05 | 0.453 | 1.80E−75 | 5.09E−70 | 2.31E−79 |
| 7-Sep | −0.3405399 | 0.214 | 0.737 | 3.48E−75 | 2.28E−52 | 4.46E−79 |
| Ech1 | −0.30879814 | 0.06 | 0.467 | 3.59E−75 | 1.12E−65 | 4.60E−79 |
| Ndufb2 | −0.27566813 | 0.05 | 0.451 | 3.84E−75 | 1.79E−66 | 4.92E−79 |
| Timm17a | −0.279687 | 0.046 | 0.443 | 4.98E−75 | 9.20E−70 | 6.38E−79 |
| Psma4 | −0.29573002 | 0.16 | 0.653 | 5.99E−75 | 6.27E−67 | 7.67E−79 |
| Ybx1 | −0.44100934 | 0.345 | 0.862 | 8.32E−75 | 3.52E−56 | 1.07E−78 |
| Mrpl18 | −0.25658189 | 0.101 | 0.547 | 1.28E−74 | 2.15E−67 | 1.64E−78 |
| Actr10 | −0.28772941 | 0.058 | 0.465 | 1.34E−74 | 4.78E−63 | 1.72E−78 |
| Mcts1 | −0.28704394 | 0.033 | 0.416 | 2.43E−74 | 3.39E−66 | 3.12E−78 |
| Tubb4b | −0.37005219 | 0.104 | 0.541 | 2.59E−74 | 1.68E−70 | 3.32E−78 |
| Myl6 | −0.4880298 | 0.556 | 0.936 | 4.42E−74 | 5.60E−57 | 5.66E−78 |
| Uqcr10 | −0.29320548 | 0.167 | 0.656 | 5.84E−74 | 7.84E−58 | 7.48E−78 |
| Rnaseh2c | −0.29566958 | 0.043 | 0.432 | 7.94E−74 | 2.29E−60 | 1.02E−77 |
| Tceb2 | −0.31853253 | 0.246 | 0.788 | 9.87E−74 | 7.15E−52 | 1.27E−77 |
| Cox6b1 | −0.33720109 | 0.259 | 0.809 | 1.04E−73 | 1.96E−62 | 1.33E−77 |
| Bsg | −0.39752289 | 0.198 | 0.7 | 3.16E−73 | 2.99E−61 | 4.05E−77 |
| Romo1 | −0.25058663 | 0.058 | 0.459 | 3.91E−73 | 5.37E−62 | 5.01E−77 |
| Atp5j2 | −0.32652635 | 0.248 | 0.799 | 4.75E−73 | 2.65E−37 | 6.09E−77 |
| Rpl36al | −0.44684749 | 0.483 | 0.934 | 1.05E−72 | 4.81E−53 | 1.35E−76 |
| Cox7c | −0.41020674 | 0.321 | 0.867 | 1.22E−72 | 4.26E−60 | 1.56E−76 |
| Bcl2a1b | −0.64580395 | 0.217 | 0.659 | 3.30E−72 | 8.45E−73 | 4.23E−76 |
| Eif3h | −0.47820533 | 0.406 | 0.904 | 4.59E−72 | 5.40E−53 | 5.89E−76 |
| H2afv | −0.32290403 | 0.137 | 0.595 | 1.51E−71 | 1.34E−54 | 1.93E−75 |
| Prmt1 | −0.28198992 | 0.054 | 0.447 | 1.63E−71 | 1.40E−55 | 2.09E−75 |
| Ppib | −0.41668834 | 0.345 | 0.887 | 3.05E−71 | 1.66E−53 | 3.91E−75 |
| Rap1b | −0.31482403 | 0.261 | 0.798 | 1.14E−70 | 1.25E−42 | 1.46E−74 |
| Clta | −0.30857164 | 0.272 | 0.825 | 3.70E−70 | 2.55E−41 | 4.74E−74 |
| Prkar1a | −0.26992509 | 0.236 | 0.768 | 1.72E−69 | 1.64E−46 | 2.20E−73 |
| Arpc4 | −0.26602372 | 0.184 | 0.674 | 2.31E−69 | 1.09E−51 | 2.97E−73 |
| Ftl1 | −0.44903622 | 0.44 | 0.93 | 3.02E−69 | 5.09E−64 | 3.87E−73 |
| Pa2g4 | −0.27246581 | 0.073 | 0.473 | 3.09E−69 | 3.82E−56 | 3.96E−73 |
| Ddx39 | −0.30287561 | 0.058 | 0.445 | 3.38E−69 | 1.24E−62 | 4.33E−73 |
| Uba52 | −0.44657706 | 0.397 | 0.905 | 7.90E−69 | 1.46E−35 | 1.01E−72 |
| Rexo2 | −0.26682714 | 0.057 | 0.44 | 2.16E−68 | 6.84E−57 | 2.77E−72 |
| Mrpl51 | −0.27107671 | 0.04 | 0.408 | 4.17E−68 | 5.20E−62 | 5.35E−72 |
| Serf2 | −0.3990418 | 0.439 | 0.917 | 2.98E−67 | 8.06E−41 | 3.82E−71 |
| Sh3bgrl3 | −0.47671616 | 0.472 | 0.914 | 3.56E−67 | 2.43E−49 | 4.56E−71 |
| Atp5h | −0.43124691 | 0.375 | 0.9 | 3.77E−67 | 9.06E−50 | 4.83E−71 |
| Dynll1 | −0.28163326 | 0.254 | 0.782 | 4.54E−67 | 1.85E−60 | 5.81E−71 |
| Cdk4 | −0.30069707 | 0.04 | 0.404 | 4.62E−67 | 4.64E−58 | 5.92E−71 |
| Hmgn2 | −0.38331554 | 0.04 | 0.402 | 7.77E−67 | 1.89E−58 | 9.96E−71 |
| Ywhaz | −0.30541484 | 0.265 | 0.799 | 1.12E−66 | 1.41E−50 | 1.43E−70 |
| Nap1l1 | −0.27249291 | 0.215 | 0.694 | 6.76E−61 | 4.33E−65 | 1.59E−70 |
| Polr3k | −0.25223385 | 0.044 | 0.409 | 1.86E−66 | 2.94E−57 | 2.39E−70 |
| C1qbp | −0.27442045 | 0.07 | 0.457 | 2.14E−66 | 1.11E−56 | 2.75E−70 |
| Higd1a | −0.25469866 | 0.198 | 0.687 | 2.39E−66 | 7.57E−53 | 3.06E−70 |
| Sub1 | −0.56279061 | 0.513 | 0.92 | 2.70E−66 | 1.08E−56 | 3.46E−70 |
| Ebna1bp2 | −0.25472245 | 0.044 | 0.409 | 3.39E−66 | 1.26E−55 | 4.35E−70 |
| Pgk1 | −0.28716854 | 0.068 | 0.442 | 4.19E−63 | 2.69E−67 | 5.91E−70 |
| Rpl31 | −0.4291132 | 0.511 | 0.923 | 8.05E−66 | 1.94E−36 | 1.03E−69 |
| Cdc42 | −0.39067026 | 0.346 | 0.877 | 1.20E−65 | 8.02E−61 | 1.53E−69 |
| Dut | −0.42376301 | 0.11 | 0.514 | 1.81E−65 | 9.38E−54 | 2.32E−69 |
| Vasp | −0.29666061 | 0.256 | 0.788 | 1.88E−65 | 5.62E−42 | 2.41E−69 |
| Anxa2 | −0.44880159 | 0.256 | 0.718 | 2.59E−65 | 2.66E−57 | 3.32E−69 |
| Cd3g | −0.4764598 | 0.568 | 0.959 | 2.93E−65 | 2.95E−63 | 3.76E−69 |
| Mrpl36 | −0.26690236 | 0.053 | 0.419 | 3.59E−65 | 4.81E−62 | 4.60E−69 |
| Rhoa | −0.32996603 | 0.311 | 0.854 | 3.99E−65 | 9.65E−44 | 5.11E−69 |
| Alyref | −0.27546954 | 0.063 | 0.439 | 7.32E−65 | 2.64E−59 | 9.38E−69 |
| Rpl41 | −0.34682941 | 0.883 | 0.982 | 1.02E−64 | 1.42E−38 | 1.31E−68 |
| Psmb8 | −0.41249953 | 0.406 | 0.896 | 1.96E−64 | 6.16E−38 | 2.51E−68 |
| Hmgb1 | −0.38928918 | 0.359 | 0.878 | 6.87E−64 | 5.24E−41 | 8.81E−68 |
| Hnrnpf | −0.307229 | 0.343 | 0.883 | 9.08E−64 | 8.58E−53 | 1.16E−67 |
| Gnai2 | −0.37917613 | 0.323 | 0.848 | 2.71E−63 | 7.24E−44 | 3.48E−67 |
| Aldoa | −0.47821299 | 0.427 | 0.875 | 2.86E−63 | 4.03E−53 | 3.67E−67 |
| Cmtm7 | −0.27508969 | 0.095 | 0.472 | 7.62E−58 | 4.88E−62 | 4.92E−67 |
| Arl6ip1 | −0.39205123 | 0.311 | 0.837 | 8.64E−63 | 1.97E−44 | 1.11E−66 |
| Cox17 | −0.27022186 | 0.175 | 0.634 | 2.39E−62 | 1.67E−51 | 3.07E−66 |
| Anp32b | −0.38380849 | 0.321 | 0.836 | 6.51E−62 | 7.41E−43 | 8.35E−66 |
| Atp5b | −0.33712519 | 0.309 | 0.828 | 1.52E−61 | 1.95E−44 | 1.95E−65 |
| Bcl2a1d | −0.51546587 | 0.164 | 0.575 | 1.56E−61 | 2.79E−65 | 1.99E−65 |
| Arf5 | −0.33036618 | 0.315 | 0.856 | 2.82E−61 | 4.02E−36 | 3.62E−65 |
| Itm2b | −0.30897176 | 0.358 | 0.907 | 2.54E−60 | 6.13E−62 | 3.26E−64 |
| H3f3b | −0.37536624 | 0.692 | 0.966 | 3.25E−60 | 1.23E−54 | 4.16E−64 |
| Hnrnpa0 | −0.2508921 | 0.288 | 0.832 | 3.64E−60 | 2.58E−47 | 4.66E−64 |
| Timm8b | −0.26121654 | 0.03 | 0.358 | 4.47E−60 | 2.36E−57 | 5.73E−64 |
| Pcbp2 | −0.26640808 | 0.292 | 0.825 | 2.66E−59 | 7.37E−58 | 3.41E−63 |
| Prelid1 | −0.29144768 | 0.241 | 0.723 | 1.67E−58 | 3.20E−44 | 2.13E−62 |
| Ostf1 | −0.34689412 | 0.352 | 0.865 | 4.84E−58 | 4.19E−42 | 6.21E−62 |
| Tuba1b | −0.44225921 | 0.137 | 0.528 | 6.30E−58 | 7.56E−41 | 8.08E−62 |
| Set | −0.29767199 | 0.301 | 0.814 | 9.31E−58 | 2.51E−47 | 1.19E−61 |
| Ppp1ca | −0.28970801 | 0.302 | 0.802 | 1.21E−57 | 2.35E−37 | 1.56E−61 |
| Plac8 | −0.64076679 | 0.268 | 0.67 | 6.11E−57 | 7.48E−47 | 7.84E−61 |
| Arpc5 | −0.2540557 | 0.286 | 0.789 | 6.63E−57 | 4.73E−36 | 8.50E−61 |
| Nkg7 | −0.51037292 | 0.709 | 0.966 | 2.72E−50 | 1.75E−54 | 1.26E−60 |
| Glipr1 | −0.26088111 | 0.064 | 0.408 | 1.60E−56 | 2.57E−53 | 2.06E−60 |
| Eif3f | −0.35086732 | 0.462 | 0.925 | 4.78E−55 | 4.51E−43 | 6.12E−59 |
| Ldha | −0.45566027 | 0.516 | 0.903 | 3.98E−54 | 4.38E−47 | 5.10E−58 |
| Ybx3 | −0.33572042 | 0.261 | 0.695 | 1.15E−51 | 1.19E−37 | 1.47E−55 |
| Ly6a | −0.50928327 | 0.283 | 0.681 | 3.27E−51 | 1.44E−38 | 4.20E−55 |
| Tubb5 | −0.3867556 | 0.311 | 0.774 | 7.12E−51 | 7.07E−35 | 9.13E−55 |
| Rac2 | −0.3774755 | 0.516 | 0.929 | 3.26E−49 | 5.35E−31 | 4.18E−53 |
| AW112010 | −0.4070269 | 0.64 | 0.96 | 4.52E−48 | 2.90E−52 | 1.39E−52 |
| Serpinb6b | −0.48993329 | 0.325 | 0.714 | 3.20E−47 | 2.81E−37 | 4.11E−51 |
| Gapdh | −0.45315475 | 0.698 | 0.95 | 1.15E−46 | 1.32E−37 | 1.48E−50 |
| Slc25a3 | −0.27119666 | 0.396 | 0.89 | 1.60E−45 | 2.99E−33 | 2.05E−49 |
| mt-Nd4l | 1.043474577 | 0.661 | 0.759 | 2.46E−45 | 8.33E−33 | 3.16E−49 |
| Cd52 | −0.4360284 | 0.728 | 0.973 | 2.61E−45 | 8.22E−45 | 3.35E−49 |
| Serbp1 | −0.30604772 | 0.456 | 0.916 | 3.86E−44 | 1.03E−44 | 4.94E−48 |
| Arpc1b | −0.32492928 | 0.434 | 0.9 | 8.32E−44 | 1.18E−27 | 1.07E−47 |
| Cox4i1 | −0.30235262 | 0.5 | 0.928 | 2.61E−43 | 2.54E−27 | 3.35E−47 |
| Klf2 | −0.32709465 | 0.217 | 0.595 | 3.12E−43 | 2.93E−23 | 4.00E−47 |
| Cyba | −0.29969003 | 0.407 | 0.883 | 1.25E−42 | 2.77E−45 | 1.61E−46 |
| H2afx | −0.2904262 | 0.023 | 0.275 | 2.63E−42 | 2.80E−37 | 3.37E−46 |
| Cd53 | −0.25726004 | 0.446 | 0.927 | 2.86E−38 | 1.83E−42 | 5.38E−46 |
| Actr3 | −0.25325219 | 0.422 | 0.89 | 3.05E−41 | 7.88E−45 | 3.91E−45 |
| S100a4 | −0.57035523 | 0.262 | 0.601 | 7.79E−41 | 1.02E−33 | 9.98E−45 |
| Cd8b1 | −0.33775466 | 0.43 | 0.821 | 1.68E−39 | 3.11E−23 | 2.16E−43 |
| Gadd45b | −0.28617261 | 0.157 | 0.484 | 1.04E−38 | 2.17E−29 | 1.33E−42 |
| Calr | −0.26248062 | 0.343 | 0.786 | 6.74E−37 | 4.46E−41 | 8.64E−41 |
| Vim | −0.38767651 | 0.393 | 0.759 | 3.24E−36 | 3.64E−19 | 4.15E−40 |
| Ifng | −1.11685705 | 0.202 | 0.511 | 2.29E−33 | 1.46E−37 | 4.59E−40 |
| Hmgb2 | −0.37392064 | 0.379 | 0.812 | 1.31E−35 | 3.46E−37 | 1.68E−39 |
| Tnfrsf9 | −0.43559203 | 0.299 | 0.64 | 1.36E−35 | 5.34E−27 | 1.74E−39 |
| Gzmb | −0.8874098 | 0.584 | 0.867 | 2.54E−34 | 1.54E−32 | 3.26E−38 |
| Lgals1 | −0.46346577 | 0.53 | 0.827 | 1.13E−33 | 7.74E−27 | 1.45E−37 |
| Prf1 | −0.29457018 | 0.15 | 0.443 | 1.95E−32 | 1.25E−36 | 2.45E−36 |
| Stmn1 | −0.50644603 | 0.06 | 0.286 | 2.57E−31 | 1.47E−32 | 3.30E−35 |
| S100a6 | −0.45042347 | 0.511 | 0.811 | 1.02E−28 | 3.05E−27 | 1.31E−32 |
| Tnfrsf4 | −0.27009629 | 0.124 | 0.369 | 3.68E−26 | 1.69E−24 | 4.72E−30 |
| Top2a | −0.33816656 | 0.08 | 0.262 | 5.57E−20 | 3.57E−24 | 2.11E−29 |
| Ggnbp2 | 0.272715946 | 0.228 | 0.575 | 5.82E−23 | 2.71E−06 | 7.46E−27 |
| Srrm2 | 0.536252955 | 0.718 | 0.923 | 6.97E−23 | 9.36E−14 | 8.94E−27 |
| Ptprc | 0.424559792 | 0.897 | 0.993 | 9.67E−23 | 7.22E−16 | 1.24E−26 |
| Ifitm2 | −0.34161017 | 0.085 | 0.244 | 2.91E−15 | 1.86E−19 | 3.88E−26 |
| Kansl1 | 0.251547382 | 0.179 | 0.472 | 9.18E−22 | 2.94E−18 | 1.18E−25 |
| Kdm2a | 0.265868798 | 0.199 | 0.515 | 1.05E−21 | 7.78E−18 | 1.34E−25 |
| Sbno1 | 0.266587862 | 0.254 | 0.618 | 2.45E−21 | 1.02E−10 | 3.14E−25 |
| Hsph1 | 0.265980118 | 0.185 | 0.475 | 4.72E−21 | 2.00E−12 | 6.05E−25 |
| Dhx36 | 0.250024767 | 0.218 | 0.541 | 9.88E−21 | 5.27E−11 | 1.27E−24 |
| Gzma | −0.81056284 | 0.162 | 0.347 | 1.79E−15 | 1.15E−19 | 1.74E−23 |
| Laptm5 | 0.44755967 | 0.798 | 0.965 | 2.42E−19 | 1.55E−23 | 1.76E−23 |
| Rbbp6 | 0.302097311 | 0.248 | 0.594 | 1.52E−19 | 7.96E−18 | 1.95E−23 |
| Ddx46 | 0.251273484 | 0.266 | 0.63 | 2.22E−19 | 6.05E−11 | 2.85E−23 |
| Ccl3 | −1.1026535 | 0.14 | 0.345 | 2.33E−19 | 2.17E−22 | 2.99E−23 |
| Brd1 | 0.254774402 | 0.212 | 0.522 | 3.62E−19 | 9.99E−18 | 4.64E−23 |
| Sltm | 0.267717046 | 0.232 | 0.56 | 5.31E−19 | 1.16E−14 | 6.81E−23 |
| Cdkn1b | 0.255824827 | 0.217 | 0.522 | 5.33E−19 | 4.81E−14 | 6.83E−23 |
| 2810417H13Rik | −0.33104242 | 0.051 | 0.195 | 6.94E−16 | 4.45E−20 | 9.58E−23 |
| Rasal3 | 0.278635074 | 0.214 | 0.521 | 1.11E−18 | 4.71E−11 | 1.42E−22 |
| Txk | 0.25327725 | 0.226 | 0.533 | 1.49E−18 | 6.60E−12 | 1.90E−22 |
| Itch | 0.252239034 | 0.152 | 0.398 | 2.75E−18 | 5.12E−17 | 3.52E−22 |
| Ubr2 | 0.263086958 | 0.174 | 0.44 | 3.93E−18 | 9.80E−16 | 5.03E−22 |
| Jmjd1c | 0.316047521 | 0.199 | 0.486 | 5.29E−18 | 3.78E−09 | 6.79E−22 |
| Krit1 | 0.329462567 | 0.249 | 0.583 | 6.00E−18 | 2.38E−14 | 7.69E−22 |
| R3hdm1 | 0.257508242 | 0.229 | 0.544 | 7.29E−18 | 1.61E−11 | 9.34E−22 |
| Nrd1 | 0.281527644 | 0.202 | 0.492 | 9.24E−18 | 6.01E−18 | 1.18E−21 |
| Acap1 | 0.258598353 | 0.178 | 0.447 | 1.19E−17 | 2.71E−10 | 1.53E−21 |
| Zc3h13 | 0.348260388 | 0.204 | 0.493 | 2.28E−17 | 1.35E−14 | 2.93E−21 |
| Zfp638 | 0.253003269 | 0.172 | 0.43 | 2.76E−17 | 1.16E−12 | 3.54E−21 |
| Setd2 | 0.283582237 | 0.177 | 0.436 | 6.96E−17 | 5.55E−12 | 8.92E−21 |
| Hist1h2ap | −0.45808245 | 0.048 | 0.195 | 7.18E−17 | 1.80E−17 | 9.20E−21 |
| Itsn2 | 0.270898623 | 0.165 | 0.416 | 9.53E−17 | 4.08E−16 | 1.22E−20 |
| Xcl1 | −0.50845392 | 0.066 | 0.226 | 9.61E−17 | 1.74E−11 | 1.23E−20 |
| Ubr5 | 0.25964204 | 0.205 | 0.489 | 2.10E−16 | 1.46E−11 | 2.69E−20 |
| Wapl | 0.284166035 | 0.261 | 0.601 | 2.82E−16 | 2.04E−14 | 3.61E−20 |
| Cdk12 | 0.28848205 | 0.211 | 0.498 | 3.32E−16 | 6.06E−09 | 4.26E−20 |
| Phf14 | 0.275996366 | 0.194 | 0.465 | 3.37E−16 | 4.53E−20 | 4.32E−20 |
| Hnrnph1 | 0.288993994 | 0.288 | 0.646 | 4.49E−16 | 6.51E−20 | 5.75E−20 |
| Rbm39 | 0.398178871 | 0.764 | 0.964 | 2.91E−15 | 1.86E−19 | 5.80E−20 |
| Suco | 0.258562568 | 0.155 | 0.389 | 4.80E−16 | 8.32E−09 | 6.16E−20 |
| Ivns1abp | 0.276268091 | 0.151 | 0.383 | 6.03E−16 | 1.83E−11 | 7.73E−20 |
| Chd8 | 0.309774654 | 0.185 | 0.445 | 1.44E−15 | 1.04E−17 | 1.84E−19 |
| Dync1h1 | 0.255451573 | 0.145 | 0.37 | 1.45E−15 | 1.79E−11 | 1.86E−19 |
| Tcf12 | 0.316293526 | 0.182 | 0.438 | 1.52E−15 | 2.14E−16 | 1.95E−19 |
| Zfp644 | 0.315582025 | 0.204 | 0.48 | 2.34E−15 | 1.53E−08 | 3.00E−19 |
| Dennd4a | 0.303682671 | 0.225 | 0.517 | 2.63E−15 | 1.33E−15 | 3.37E−19 |
| Huwe1 | 0.303390284 | 0.231 | 0.531 | 3.09E−15 | 2.20E−14 | 3.96E−19 |
| Arid4a | 0.318574015 | 0.246 | 0.56 | 3.23E−15 | 3.20E−15 | 4.14E−19 |
| Herc1 | 0.288173346 | 0.209 | 0.484 | 4.52E−15 | 1.85E−06 | 5.79E−19 |
| Arglu1 | 0.312874795 | 0.261 | 0.59 | 8.64E−15 | 0.0001526 | 1.11E−18 |
| Snrnp70 | 0.317034965 | 0.259 | 0.586 | 1.04E−14 | 2.22E−12 | 1.33E−18 |
| Parp14 | 0.252600339 | 0.148 | 0.367 | 1.04E−14 | 7.62E−08 | 1.33E−18 |
| Fnbp4 | 0.335157046 | 0.179 | 0.428 | 1.07E−14 | 2.55E−10 | 1.37E−18 |
| Raf1 | 0.261288692 | 0.155 | 0.381 | 1.24E−14 | 7.94E−19 | 1.42E−18 |
| Jun | 0.330597999 | 0.215 | 0.476 | 1.26E−14 | 4.66E−12 | 1.61E−18 |
| Hnrnpl | 0.344362072 | 0.248 | 0.564 | 1.41E−14 | 2.56E−08 | 1.81E−18 |
| Usp48 | 0.334353422 | 0.205 | 0.475 | 1.63E−14 | 4.23E−08 | 2.09E−18 |
| Pstpip1 | 0.251998016 | 0.252 | 0.567 | 1.96E−14 | 9.18E−05 | 2.51E−18 |
| Srrt | 0.272059409 | 0.178 | 0.422 | 2.07E−14 | 7.04E−14 | 2.65E−18 |
| Clcn3 | 0.287336332 | 0.179 | 0.423 | 2.12E−14 | 2.80E−17 | 2.72E−18 |
| Atp11b | 0.30754502 | 0.299 | 0.64 | 2.55E−12 | 1.64E−16 | 2.79E−18 |
| Ranbp2 | 0.271667267 | 0.195 | 0.452 | 3.82E−14 | 4.22E−11 | 4.90E−18 |
| Mkln1 | 0.314061187 | 0.178 | 0.418 | 5.51E−14 | 1.35E−13 | 7.06E−18 |
| Gpr132 | 0.284445049 | 0.208 | 0.472 | 6.33E−14 | 1.62E−14 | 8.12E−18 |
| Tia1 | 0.284645792 | 0.15 | 0.365 | 7.11E−14 | 1.13E−12 | 9.11E−18 |
| Rif1 | 0.373719942 | 0.232 | 0.471 | 1.49E−07 | 9.52E−12 | 1.17E−17 |
| Zfc3h1 | 0.261110294 | 0.114 | 0.301 | 1.03E−13 | 1.28E−13 | 1.32E−17 |
| Slc1a5 | 0.303892569 | 0.224 | 0.502 | 1.06E−13 | 1.63E−12 | 1.36E−17 |
| Ssh2 | 0.259074714 | 0.256 | 0.555 | 1.11E−13 | 1.39E−10 | 1.43E−17 |
| Ogt | 0.280264739 | 0.321 | 0.699 | 1.19E−13 | 1.28E−08 | 1.53E−17 |
| Phf20l1 | 0.295128155 | 0.228 | 0.514 | 1.22E−13 | 2.05E−07 | 1.57E−17 |
| Map4k4 | 0.328013398 | 0.248 | 0.548 | 1.50E−13 | 2.77E−11 | 1.92E−17 |
| Asxl2 | 0.303138204 | 0.162 | 0.388 | 1.65E−13 | 8.42E−16 | 2.11E−17 |
| Tet2 | 0.251179227 | 0.132 | 0.332 | 1.88E−13 | 1.21E−06 | 2.41E−17 |
| Pan3 | 0.258048781 | 0.135 | 0.336 | 2.20E−13 | 4.67E−06 | 2.82E−17 |
| Nufip2 | 0.359606211 | 0.214 | 0.483 | 2.27E−13 | 7.23E−10 | 2.91E−17 |
| Hdac7 | 0.325524672 | 0.199 | 0.457 | 2.37E−13 | 2.81E−07 | 3.04E−17 |
| Prrc2a | 0.318679785 | 0.212 | 0.482 | 3.18E−13 | 1.71E−13 | 4.07E−17 |
| Gm26917 | 1.387712099 | 0.521 | 0.716 | 0.637272 | 4.08E−05 | 4.71E−17 |
| Pde7a | 0.309157935 | 0.245 | 0.539 | 4.15E−13 | 7.84E−08 | 5.32E−17 |
| Trpm7 | 0.357677265 | 0.179 | 0.413 | 1.69E−12 | 1.08E−16 | 7.27E−17 |
| Gcc2 | 0.315440829 | 0.147 | 0.343 | 1.35E−11 | 8.64E−16 | 7.70E−17 |
| Ccnt2 | 0.261078658 | 0.137 | 0.336 | 6.70E−13 | 6.77E−13 | 8.60E−17 |
| Rasa1 | 0.302392018 | 0.184 | 0.423 | 8.41E−13 | 2.28E−16 | 1.08E−16 |
| Upf2 | 0.27994868 | 0.131 | 0.325 | 9.57E−13 | 3.54E−15 | 1.23E−16 |
| Atf7ip | 0.338872248 | 0.229 | 0.507 | 1.17E−12 | 1.84E−08 | 1.50E−16 |
| Ttc3 | 0.270475173 | 0.142 | 0.343 | 1.22E−12 | 9.84E−13 | 1.57E−16 |
| Nfat5 | 0.274057639 | 0.219 | 0.481 | 1.71E−12 | 9.66E−08 | 2.20E−16 |
| Trmt1l | 0.255618744 | 0.114 | 0.293 | 1.74E−12 | 1.16E−10 | 2.23E−16 |
| Adgre5 | 0.263330101 | 0.212 | 0.468 | 1.86E−12 | 1.42E−12 | 2.38E−16 |
| Pcf11 | 0.325303885 | 0.185 | 0.42 | 1.87E−12 | 6.81E−12 | 2.40E−16 |
| Itpkb | 0.309725775 | 0.333 | 0.658 | 4.30E−08 | 2.76E−12 | 2.96E−16 |
| Eif4g3 | 0.379167635 | 0.234 | 0.516 | 2.61E−12 | 2.78E−15 | 3.35E−16 |
| Peak1 | 0.381813096 | 0.229 | 0.464 | 1.60E−07 | 1.03E−11 | 3.56E−16 |
| Rexo1 | 0.357060067 | 0.16 | 0.374 | 3.90E−12 | 1.04E−13 | 4.99E−16 |
| Cep250 | 0.284232879 | 0.142 | 0.341 | 4.62E−12 | 4.06E−11 | 5.92E−16 |
| Tmem259 | 0.280227589 | 0.135 | 0.326 | 5.45E−12 | 3.38E−12 | 6.99E−16 |
| Pik3cd | 0.340287653 | 0.292 | 0.633 | 5.53E−12 | 4.86E−06 | 7.09E−16 |
| Phf3 | 0.276872218 | 0.313 | 0.669 | 5.95E−12 | 3.38E−13 | 7.63E−16 |
| Madd | 0.360373918 | 0.244 | 0.528 | 6.45E−12 | 2.94E−05 | 8.27E−16 |
| Tmc6 | 0.301407584 | 0.123 | 0.304 | 6.66E−12 | 2.21E−08 | 8.54E−16 |
| Golga4 | 0.350764725 | 0.219 | 0.484 | 7.45E−12 | 4.51E−09 | 9.55E−16 |
| Nisch | 0.403455071 | 0.236 | 0.515 | 1.07E−11 | 6.55E−08 | 1.38E−15 |
| Ccl4 | −1.16370543 | 0.372 | 0.615 | 1.11E−11 | 3.53E−12 | 1.42E−15 |
| Nbeal2 | 0.26862623 | 0.148 | 0.347 | 1.21E−11 | 5.17E−07 | 1.55E−15 |
| Dmtf1 | 0.282837423 | 0.147 | 0.345 | 1.51E−11 | 6.10E−10 | 1.94E−15 |
| Spag9 | 0.274730828 | 0.231 | 0.497 | 1.61E−11 | 5.01E−15 | 2.07E−15 |
| Slc30a9 | 0.292578438 | 0.124 | 0.304 | 1.62E−11 | 1.13E−09 | 2.08E−15 |
| Jarid2 | 0.299968088 | 0.185 | 0.413 | 1.75E−11 | 1.61E−12 | 2.25E−15 |
| Vcpip1 | 0.254795472 | 0.11 | 0.278 | 1.75E−11 | 5.70E−12 | 2.25E−15 |
| Ythdc1 | 0.360037083 | 0.282 | 0.609 | 2.20E−11 | 5.97E−09 | 2.81E−15 |
| Kif21b | 0.271429749 | 0.182 | 0.406 | 2.70E−11 | 5.18E−14 | 3.45E−15 |
| Ccl5 | −0.34711884 | 0.812 | 0.927 | 3.08E−11 | 1.50E−10 | 3.95E−15 |
| Git2 | 0.388424312 | 0.279 | 0.561 | 1.21E−07 | 7.74E−12 | 4.64E−15 |
| Hivep2 | 0.28779872 | 0.132 | 0.3 | 5.01E−09 | 3.21E−13 | 4.66E−15 |
| Zzef1 | 0.324073528 | 0.145 | 0.338 | 4.15E−11 | 1.69E−10 | 5.33E−15 |
| Tet3 | 0.323555021 | 0.158 | 0.356 | 2.41E−10 | 1.54E−14 | 5.53E−15 |
| Ikzf3 | 0.351844821 | 0.199 | 0.431 | 1.99E−10 | 1.28E−14 | 9.15E−15 |
| Ip6k1 | 0.250622346 | 0.11 | 0.274 | 7.54E−11 | 1.37E−08 | 9.67E−15 |
| Ankrd11 | 0.599872171 | 0.631 | 0.868 | 8.24E−11 | 4.52E−13 | 1.06E−14 |
| Chd3 | 0.361743498 | 0.236 | 0.506 | 9.31E−11 | 7.32E−08 | 1.19E−14 |
| Tcf25 | 0.331535334 | 0.313 | 0.673 | 9.46E−11 | 1.33E−13 | 1.21E−14 |
| Hectd1 | 0.314010332 | 0.316 | 0.672 | 1.09E−10 | 1.57E−12 | 1.40E−14 |
| Gzmc | −0.54134656 | 0.058 | 0.168 | 5.25E−09 | 3.37E−13 | 2.00E−14 |
| Arid4b | 0.330190435 | 0.311 | 0.654 | 1.94E−10 | 5.30E−10 | 2.48E−14 |
| Suv420h1 | 0.293338552 | 0.155 | 0.351 | 2.08E−10 | 2.52E−11 | 2.67E−14 |
| Paxbp1 | 0.308962869 | 0.118 | 0.286 | 2.08E−10 | 1.04E−11 | 2.67E−14 |
| Fryl | 0.388285685 | 0.236 | 0.504 | 2.18E−10 | 1.60E−12 | 2.79E−14 |
| BC005537 | 0.323029831 | 0.283 | 0.602 | 2.35E−10 | 1.10E−05 | 3.01E−14 |
| Dennd1c | 0.281187336 | 0.124 | 0.296 | 2.78E−10 | 1.14E−05 | 3.56E−14 |
| Baz2a | 0.386783669 | 0.215 | 0.457 | 1.75E−09 | 1.12E−13 | 3.66E−14 |
| Akap13 | 0.467378477 | 0.677 | 0.908 | 3.78E−10 | 3.78E−10 | 4.85E−14 |
| Atp8b4 | 0.290627081 | 0.275 | 0.568 | 3.90E−10 | 1.83E−11 | 4.99E−14 |
| Bod1l | 0.34993944 | 0.298 | 0.625 | 5.14E−10 | 0.000243 | 6.59E−14 |
| Tle4 | 0.310146011 | 0.14 | 0.321 | 6.24E−10 | 4.42E−14 | 8.00E−14 |
| Fam208a | 0.28780993 | 0.117 | 0.278 | 8.95E−10 | 4.22E−12 | 1.15E−13 |
| Galnt6 | 0.350431149 | 0.198 | 0.425 | 1.21E−09 | 2.37E−08 | 1.56E−13 |
| Smg6 | 0.337454783 | 0.189 | 0.408 | 1.24E−09 | 1.50E−09 | 1.59E−13 |
| Sytl2 | 0.424906198 | 0.191 | 0.324 | 0.512352 | 3.28E−05 | 2.20E−13 |
| AU020206 | 0.319045412 | 0.204 | 0.432 | 1.75E−09 | 0.0003175 | 2.24E−13 |
| Thoc2 | 0.367052214 | 0.301 | 0.631 | 1.85E−09 | 0.0002528 | 2.38E−13 |
| Map4k2 | 0.366000874 | 0.171 | 0.372 | 1.95E−09 | 6.62E−11 | 2.50E−13 |
| Nlrc3 | 0.325238267 | 0.152 | 0.337 | 1.97E−09 | 3.84E−10 | 2.52E−13 |
| Ubn2 | 0.409364392 | 0.218 | 0.459 | 2.54E−09 | 2.06E−11 | 3.26E−13 |
| Samd9l | 0.2590342 | 0.151 | 0.332 | 3.54E−09 | 2.61E−07 | 4.54E−13 |
| Safb2 | 0.374512352 | 0.268 | 0.559 | 3.80E−09 | 1.19E−06 | 4.87E−13 |
| Mxd4 | 0.384090913 | 0.197 | 0.378 | 4.31E−05 | 2.76E−09 | 7.33E−13 |
| Pnisr | 0.365617087 | 0.293 | 0.604 | 6.28E−09 | 4.11E−08 | 8.05E−13 |
| Prpf38b | 0.314654174 | 0.345 | 0.712 | 8.10E−09 | 0.0609514 | 1.04E−12 |
| Ppp1r10 | 0.354634324 | 0.148 | 0.327 | 8.44E−09 | 9.46E−13 | 1.08E−12 |
| Son | 0.490246027 | 0.591 | 0.876 | 0.010422 | 6.68E−07 | 1.30E−12 |
| Slc20a1 | 0.354480303 | 0.17 | 0.362 | 1.05E−08 | 4.45E−08 | 1.34E−12 |
| Cdk11b | 0.399268468 | 0.285 | 0.587 | 1.51E−08 | 3.75E−09 | 1.93E−12 |
| Nsd1 | 0.427957581 | 0.264 | 0.533 | 4.74E−07 | 3.04E−11 | 2.00E−12 |
| Kdm5a | 0.385999485 | 0.296 | 0.61 | 1.74E−08 | 5.03E−09 | 2.22E−12 |
| Atp2b1 | 0.329002596 | 0.338 | 0.684 | 1.91E−08 | 1.80E−06 | 2.45E−12 |
| Ankrd17 | 0.364127148 | 0.283 | 0.582 | 2.23E−08 | 2.61E−07 | 2.86E−12 |
| Nabp1 | 0.264149668 | 0.33 | 0.664 | 2.37E−08 | 6.92E−05 | 3.04E−12 |
| Fcho2 | 0.281645979 | 0.138 | 0.304 | 2.63E−08 | 5.56E−12 | 3.37E−12 |
| Rsbn1l | 0.398984222 | 0.312 | 0.619 | 1.27E−05 | 8.12E−10 | 6.03E−12 |
| Chd6 | 0.338933179 | 0.158 | 0.337 | 5.06E−08 | 5.03E−11 | 6.49E−12 |
| Smchd1 | 0.322663448 | 0.37 | 0.746 | 5.08E−08 | 0.0050354 | 6.51E−12 |
| Vps54 | 0.364164893 | 0.226 | 0.451 | 9.30E−07 | 5.96E−11 | 7.10E−12 |
| Grina | 0.335779567 | 0.177 | 0.369 | 8.65E−08 | 1.96E−08 | 1.11E−11 |
| Fus | 0.266206396 | 0.403 | 0.804 | 9.85E−08 | 1.79E−09 | 1.26E−11 |
| Golgb1 | 0.380037072 | 0.207 | 0.421 | 1.12E−07 | 1.42E−06 | 1.43E−11 |
| Kmt2c | 0.375029026 | 0.184 | 0.379 | 1.18E−07 | 7.12E−06 | 1.51E−11 |
| Zc3h7a | 0.557347385 | 0.281 | 0.464 | 1 | 0.0016164 | 1.64E−11 |
| Rb1cc1 | 0.39510291 | 0.279 | 0.563 | 1.49E−07 | 2.12E−11 | 1.92E−11 |
| Kcnq1ot1 | 0.299305169 | 0.254 | 0.489 | 2.06E−07 | 9.08E−08 | 2.64E−11 |
| Ccnl2 | 0.411772913 | 0.288 | 0.582 | 2.17E−07 | 0.003168 | 2.79E−11 |
| Ttc14 | 0.357378845 | 0.311 | 0.63 | 2.86E−07 | 1.76E−07 | 3.66E−11 |
| Gripap1 | 0.308060148 | 0.127 | 0.277 | 2.91E−07 | 1.87E−09 | 3.73E−11 |
| Smg1 | 0.448531515 | 0.251 | 0.507 | 2.99E−07 | 4.87E−06 | 3.84E−11 |
| Atrx | 0.639494131 | 0.578 | 0.808 | 3.28E−07 | 2.59E−06 | 4.20E−11 |
| Hip1 | 0.38083048 | 0.172 | 0.354 | 4.03E−07 | 2.59E−11 | 5.17E−11 |
| Mgea5 | 0.413571578 | 0.242 | 0.489 | 4.55E−07 | 7.91E−07 | 5.83E−11 |
| Arhgap15 | 0.404025071 | 0.336 | 0.64 | 0.001598 | 1.02E−07 | 6.48E−11 |
| Ktn1 | 0.346908055 | 0.191 | 0.376 | 5.14E−06 | 3.29E−10 | 9.96E−11 |
| Mbnl1 | 0.337176183 | 0.704 | 0.933 | 1.12E−06 | 4.55E−09 | 1.43E−10 |
| Gbp8 | 0.318234731 | 0.134 | 0.283 | 1.15E−06 | 6.71E−05 | 1.47E−10 |
| Neurl3 | 0.406158876 | 0.318 | 0.563 | 0.066267 | 4.25E−06 | 2.13E−10 |
| Phip | 0.356352972 | 0.268 | 0.527 | 2.23E−06 | 2.45E−08 | 2.86E−10 |
| Nipbl | 0.374497585 | 0.332 | 0.661 | 3.66E−06 | 6.24E−06 | 4.69E−10 |
| Bcl11b | 0.400040045 | 0.318 | 0.618 | 1.44E−05 | 9.23E−10 | 5.97E−10 |
| Zcchc11 | 0.359045991 | 0.376 | 0.721 | 0.000258 | 1.66E−08 | 8.35E−10 |
| Dusp5 | 0.338451539 | 0.328 | 0.621 | 8.09E−06 | 8.41E−06 | 1.04E−09 |
| Gigyf1 | 0.353725733 | 0.162 | 0.325 | 8.12E−06 | 6.12E−07 | 1.04E−09 |
| Ccdc88b | 0.354497455 | 0.167 | 0.334 | 9.06E−06 | 5.24E−06 | 1.16E−09 |
| Rbm5 | 0.39707282 | 0.339 | 0.668 | 1.02E−05 | 4.03E−06 | 1.30E−09 |
| Trip11 | 0.434809923 | 0.275 | 0.537 | 1.36E−05 | 2.05E−07 | 1.75E−09 |
| Rrbp1 | 0.347782832 | 0.306 | 0.591 | 1.52E−05 | 6.73E−06 | 1.95E−09 |
| Nlrc5 | 0.432228409 | 0.242 | 0.471 | 1.69E−05 | 1.38E−09 | 2.16E−09 |
| Adgrg5 | 0.444342265 | 0.167 | 0.293 | 0.083728 | 5.37E−06 | 2.24E−09 |
| Clec2d | 0.360659045 | 0.36 | 0.701 | 2.13E−05 | 0.5863519 | 2.73E−09 |
| Plec | 0.427274854 | 0.212 | 0.393 | 0.001414 | 9.06E−08 | 2.83E−09 |
| Setbp1 | 0.366950408 | 0.15 | 0.279 | 0.003509 | 2.25E−07 | 2.90E−09 |
| Tnrc6a | 0.423806771 | 0.268 | 0.522 | 2.49E−05 | 1.42E−08 | 3.19E−09 |
| Gramd1a | 0.505709215 | 0.259 | 0.504 | 2.81E−05 | 0.5538424 | 3.61E−09 |
| Tra2a | 0.331117127 | 0.379 | 0.74 | 4.10E−05 | 6.96E−08 | 5.26E−09 |
| Gm26740 | 0.391831407 | 0.224 | 0.423 | 4.53E−05 | 0.0068501 | 5.81E−09 |
| Tra2b | 0.303242989 | 0.385 | 0.755 | 4.82E−05 | 4.39E−05 | 6.18E−09 |
| Tnrc6b | 0.41240144 | 0.316 | 0.608 | 5.07E−05 | 9.14E−09 | 6.49E−09 |
| Zcchc7 | 0.424875315 | 0.282 | 0.544 | 8.56E−05 | 5.17E−05 | 1.10E−08 |
| Prpf4b | 0.448009815 | 0.309 | 0.601 | 0.000118 | 1.13E−08 | 1.51E−08 |
| Vgll4 | 0.375728611 | 0.382 | 0.728 | 0.000151 | 3.99E−08 | 1.94E−08 |
| 2810474O19Rik | 0.367499595 | 0.379 | 0.72 | 0.000162 | 0.0267953 | 2.08E−08 |
| Ash1l | 0.43221354 | 0.302 | 0.578 | 0.000167 | 1.01E−05 | 2.14E−08 |
| Baz1a | 0.26039361 | 0.397 | 0.719 | 0.000271 | 0.0515835 | 3.47E−08 |
| Mirt1 | 0.378877922 | 0.162 | 0.29 | 0.021662 | 1.39E−06 | 4.30E−08 |
| Ifitm1 | −0.41382207 | 0.181 | 0.27 | 0.167201 | 1.07E−05 | 7.62E−08 |
| Rinl | 0.400086486 | 0.359 | 0.689 | 0.000623 | 0.2712399 | 7.99E−08 |
| Erdr1 | 0.491568087 | 0.279 | 0.524 | 0.000766 | 0.250148 | 9.82E−08 |
| Akap9 | 0.485680054 | 0.292 | 0.552 | 0.000795 | 4.75E−07 | 1.02E−07 |
| Il18rap | 0.376564107 | 0.329 | 0.611 | 0.00097 | 0.0001538 | 1.24E−07 |
| Itga4 | 0.37300617 | 0.265 | 0.451 | 0.051146 | 3.28E−06 | 1.49E−07 |
| Tnrc6c | 0.457508107 | 0.276 | 0.516 | 0.002085 | 0.0013647 | 2.67E−07 |
| Cntrl | 0.391550206 | 0.359 | 0.683 | 0.00443 | 4.95E−05 | 5.68E−07 |
| Mndal | 0.348373647 | 0.379 | 0.704 | 0.005956 | 8.39E−05 | 7.63E−07 |
| Dock2 | 0.315799741 | 0.41 | 0.778 | 0.014488 | 0.0120678 | 1.86E−06 |
| Rbm25 | 0.258617939 | 0.44 | 0.826 | 0.026674 | 0.8219128 | 3.42E−06 |
| Fyb | 0.408348413 | 0.531 | 0.856 | 1 | 0.9616142 | 3.76E−06 |
| Satb1 | 0.364106027 | 0.509 | 0.818 | 1 | 0.1588182 | 5.18E−06 |
| Macf1 | 0.715036314 | 0.516 | 0.726 | 0.050829 | 0.0001013 | 6.52E−06 |
| Ikzf2 | 0.64913581 | 0.299 | 0.425 | 1 | 0.063428 | 9.27E−06 |
| Bptf | 0.363135027 | 0.393 | 0.737 | 0.079326 | 0.0006198 | 1.02E−05 |
| Rapgef6 | 0.345997092 | 0.407 | 0.741 | 0.566213 | 3.63E−05 | 1.17E−05 |
| PISD | 0.493486264 | 0.221 | 0.386 | 0.105222 | 0.0061199 | 1.35E−05 |
| Arap2 | 0.429803078 | 0.346 | 0.616 | 0.485938 | 3.11E−05 | 2.16E−05 |
| Neb | 0.395368858 | 0.083 | 0.136 | 1 | 0.0011273 | 2.19E−05 |
| Arhgef1 | 0.433285357 | 0.349 | 0.644 | 0.178974 | 0.0235464 | 2.29E−05 |
| mt-Atp8 | 0.831701822 | 0.403 | 0.49 | 0.186229 | 0.0704002 | 2.39E−05 |
| Ahnak | 0.575000378 | 0.48 | 0.728 | 1 | 0.3571544 | 2.69E−05 |
| Gzmf | −0.96815579 | 0.11 | 0.183 | 0.235036 | 0.000162 | 3.01E−05 |
| H2-Q4 | 0.344713945 | 0.433 | 0.782 | 1 | 0.0001013 | 3.06E−05 |
| Lars2 | 1.020618306 | 0.348 | 0.405 | 1 | 0.0002078 | 3.55E−05 |
| Bcl2 | 0.423516103 | 0.415 | 0.667 | 0.290997 | 1.94E−05 | 3.73E−05 |
| Usp34 | 0.470590127 | 0.35 | 0.641 | 0.395855 | 0.0001012 | 5.07E−05 |
| 4932438A13Rik | 0.637509223 | 0.397 | 0.608 | 1 | 0.889755 | 0.000101 |
| AY036118 | 0.481144098 | 0.177 | 0.298 | 0.813161 | 0.026985 | 0.000104 |
| Smad7 | 0.62162811 | 0.423 | 0.673 | 1 | 0.3640972 | 0.000137 |
| Dgat1 | 0.525033621 | 0.295 | 0.493 | 1 | 0.0138039 | 0.000492 |
| Birc6 | 0.441922518 | 0.376 | 0.676 | 1 | 0.0006307 | 0.000619 |
| Kmt2e | 0.459272684 | 0.53 | 0.836 | 1 | 0.2295868 | 0.000619 |
| Tecpr1 | 0.424010914 | 0.38 | 0.663 | 1 | 0.9388163 | 0.000777 |
| Hmha1 | 0.588586119 | 0.526 | 0.783 | 1 | 0.0072321 | 0.000808 |
| Ankrd12 | 0.457078518 | 0.366 | 0.634 | 1 | 0.0012742 | 0.000988 |
| Lck | 0.337663705 | 0.544 | 0.869 | 1 | 0.2133148 | 0.001863 |
| Arhgap30 | 0.346157238 | 0.406 | 0.718 | 1 | 0.0016413 | 0.002107 |
| Arid5a | 0.451447806 | 0.36 | 0.618 | 1 | 0.0273972 | 0.002819 |
| Lyst | 0.578848075 | 0.343 | 0.544 | 1 | 0.03666 | 0.003884 |
| Dock10 | 0.357407935 | 0.425 | 0.755 | 1 | 0.0031917 | 0.003949 |
| Ccnl1 | 0.41957009 | 0.412 | 0.729 | 1 | 0.1829923 | 0.00478 |
| Sf3b1 | 0.344481674 | 0.558 | 0.878 | 1 | 0.2444548 | 0.004924 |
| Neat1 | 0.56729499 | 0.302 | 0.495 | 1 | 0.0902443 | 0.006318 |
| H2-Q6 | 0.396977307 | 0.446 | 0.768 | 1 | 0.1287671 | 0.00819 |
| Btg2 | 0.319169469 | 0.484 | 0.814 | 1 | 0.0136522 | 0.013101 |
| Rsrp1 | 0.362269613 | 0.581 | 0.867 | 1 | 0.1941922 | 0.014434 |
| Chd7 | 0.536244478 | 0.462 | 0.734 | 1 | 0.82985 | 0.017192 |
| Clk1 | 0.445331045 | 0.452 | 0.784 | 1 | 0.0646196 | 0.038014 |
| Ncor1 | 0.359731768 | 0.469 | 0.827 | 1 | 0.0313663 | 0.051813 |
| Chd2 | 0.524354375 | 0.39 | 0.649 | 1 | 0.052343 | 0.056611 |
| Wnk1 | 0.342204992 | 0.53 | 0.839 | 1 | 0.8397891 | 0.060685 |
| Ankrd44 | 0.499118893 | 0.406 | 0.695 | 1 | 0.8141186 | 0.061926 |
| Prrc2c | 0.359695418 | 0.577 | 0.889 | 1 | 0.9766777 | 0.090375 |
| Utrn | 0.695602508 | 0.44 | 0.648 | 1 | 0.2286153 | 0.095361 |
| Nktr | 0.607668623 | 0.481 | 0.731 | 1 | 0.3132434 | 0.13155 |
| Luc7l2 | 0.447924496 | 0.528 | 0.833 | 1 | 0.299566 | 0.156017 |
| mt-Nd6 | 0.547918393 | 0.162 | 0.177 | 1 | 0.4863416 | 0.261748 |
| Whsc1l1 | 0.43335585 | 0.486 | 0.808 | 1 | 0.3109598 | 0.435287 |
| Zfp292 | 0.486279942 | 0.45 | 0.747 | 1 | 0.3663029 | 0.480136 |
| Mycbp2 | 0.552422571 | 0.456 | 0.743 | 1 | 0.9721877 | 0.554879 |
| Iqgap1 | 0.301529834 | 0.511 | 0.839 | 1 | 0.4691463 | 0.682641 |
| Kmt2a | 0.64166127 | 0.43 | 0.702 | 1 | 0.8827486 | 0.986162 |

### (3) Male Bias in CD8⁺ Progenitor Exhausted T Cell Frequency in the Tumor Microenvironment

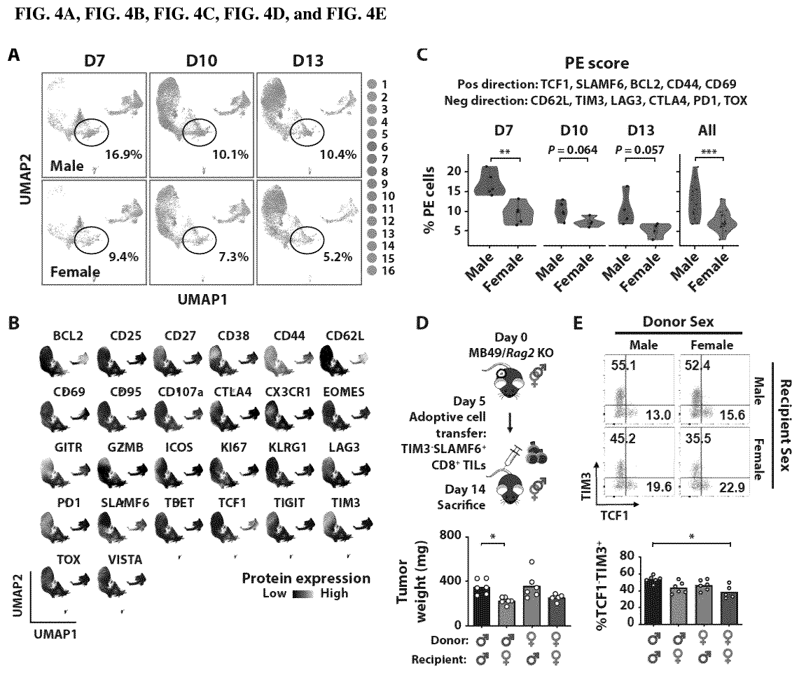

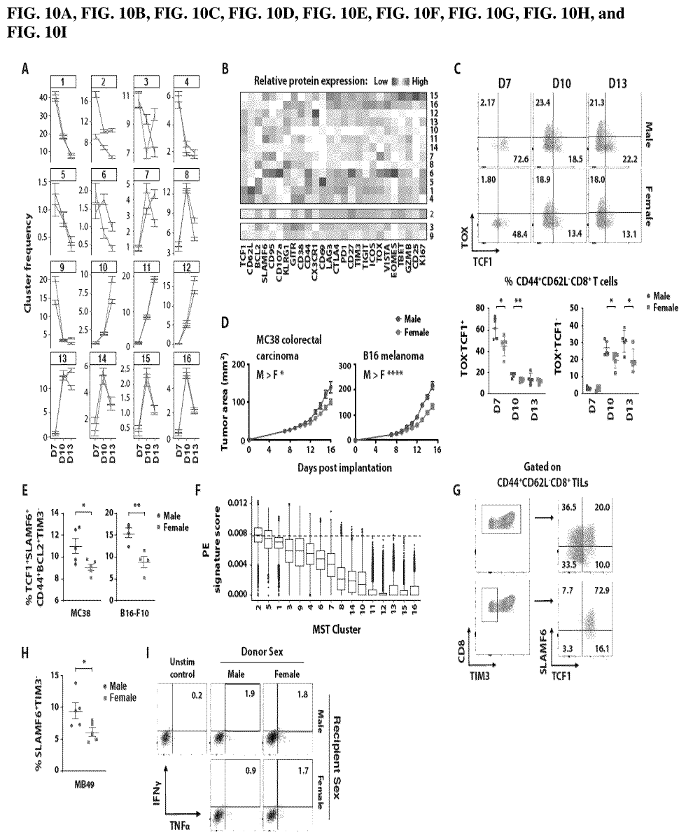

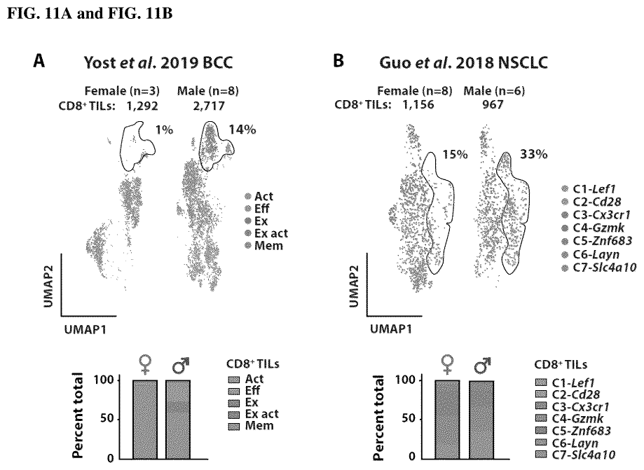

We next used high dimensional spectral flow cytometry to validate and further investigate sex differences in CD8⁺ TIL fate before (Day 7), during (Day 10) and after (Day 13) the sex-based bifurcation in MB49 growth. By performing single cell analyses with 26 key T cell markers and dimensional reduction with UMAP, we identified 16 major clusters of cells at various stages of CD8⁺ T cell differentiation (FIGS. 4, A and B, FIGS. 10A and 10B). On Day 7, the tumors were highly enriched with TCF1⁺BCL2⁺SLAMF6⁺CD62L⁺ cells in cluster 1, which are reminiscent of primed T cells that have just migrated into the tumor. By Days 10 and 13, there was a gradual accumulation of activated T cells from their cognate tumor antigen recognition. Importantly, only cluster 2, which expressed TCF1, BCL2 and SLAMF6, showed the male-biased frequency that was most prominent on Day 7 but consistent throughout all examined time points (FIG. 10A). The expression profile of cluster 2 from spectral flow cytometry closely resembled CD8⁺ PE T cells from the scRNA-seq analyses with the exception of Sell (CD62L), which can be a consequence of its unique transcriptional or post-translational regulation. By exclusively focusing on TCF1 and TOX expression in the CD44⁺CD62L⁻ subset, we observed the male-biased frequency of TCF1⁺ cells, which later manifested as TOX⁺ cells (FIG. 10C). Similarly, we noted a higher proportion of PE-associated protein expression in male versus female MC38 colon cancer and B16-F10 melanoma, both of which demonstrated an aggressive male-biased tumor growth (FIGS. 10D and 10E). Using UCell, a robust single-cell gene signature scoring method, we also developed a protein-level PE signature based on markers that are collectively known to be more positively (TCF1, SLAMF6, BCL2, CD44, CD69) or negatively (CD62L, PD1, LAG3, TIM3, CTLA4, TOX) expressed in PE cells compared to other CD8⁺ T cell phenotypes. The PE signature identified cluster 2 as the primary PE population (FIG. 10F) and, consistent with our scRNA-seq results (FIG. 3, A to D), demonstrated a higher CD8⁺ PE TIL frequency in male versus female tumors (FIG. 4C). Of note, PD1 is expressed at low levels in the cluster 2 PE cell population compared to activated CD8⁺ T cells. Overall, we conclude that the male tumor microenvironment favors the development of CD8⁺TCF1⁺ PE T cells.

An important question is whether these CD44⁺CD62L⁻TCF1⁺CD8⁺ T cells indeed have the progenitor capacity to continue to differentiate within the tumor microenvironment. To address this question, we utilized an adoptive transfer model, in which we tracked the fate of these cells in Rag2 KO mice using SLAMF6 as a cell surface surrogate for TCF1 expression (FIG. 4D). Consistent with the literature, SLAMF6 expression in CD44⁺CD62L⁻TIM3⁻CD8⁺ T cells correlated well with TCF1 (FIG. 10G). Similarly, we were able to detect a male-biased frequency of PE cells based on SLAMF6 and TIM3 expression alone (FIG. 10H). Donor SLAMF6⁺TIM3⁻CD8⁺ TILs gave rise to TCF1⁻TIM3⁺ bonafide exhausted T cells that failed to respond to re-stimulation with PMA/Ionomycin (FIG. 10I), indicating a clear precursor-product relationship. Importantly, tumor control was worse in males after either male or female cells were adoptively transferred, and the frequency of these terminally exhausted T cells was higher in male versus female recipient tumors (FIGS. 4, D and E). These findings indicate that a male-biased formation of CD8⁺ PE TILs eventually leads to more functionally exhausted T cells in male mice and contributes to poorer control of tumors in male versus female host. We sought to extend our analyses and assess whether a male bias in CD8⁺ T cell exhaustion was also present in human cancer. Following the same T cell state annotations, we observed a male-biased frequency of terminally exhausted CD8⁺ TILs in both BCC (“CD8_Ex”; FIG. 11A) and treatment-naïve NSCLC (“CD8_C6-Layn”; FIG. 11B).

### (4) Roles of Androgen in Mediating Sex Bias in T Cell Exhaustion

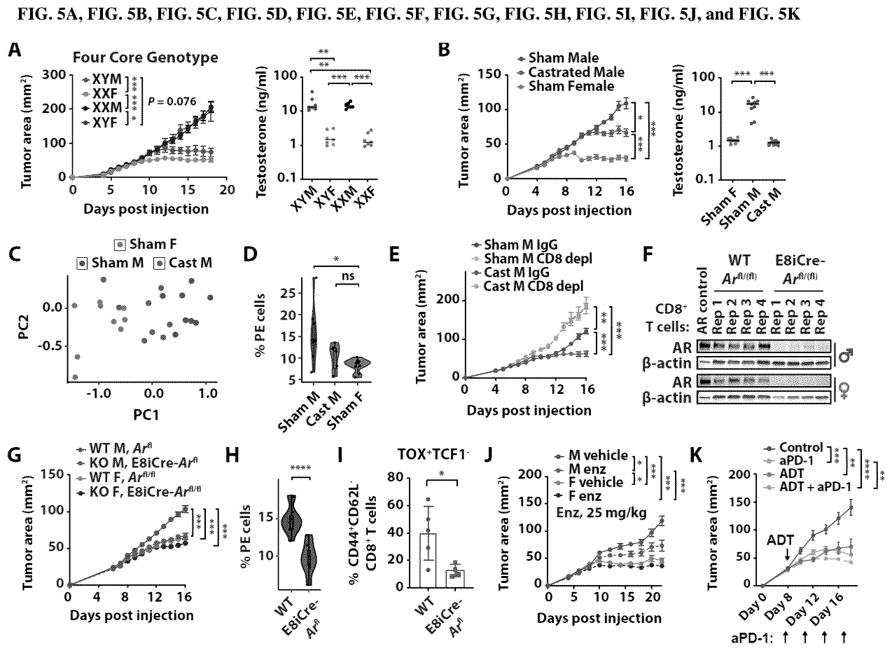

We used the Four Core Genotype mouse model to determine whether male-biased CD8⁺ T cell exhaustion is driven by sex chromosomes or sex hormones. We found that MB49 grew most aggressively in mice with high levels of testosterone regardless of sex chromosome compositions, indicating that pertinent sex-specific differences in CD8⁺ T cell immunity are regulated by androgen (FIG. 5A). We next performed a series of surgical, genetic and pharmacological experiments to evaluate how disrupting the androgen-AR axis would affect tumor growth and intratumoral CD8⁺ T cell dynamics. First, we castrated male mice to decrease the levels of circulating testosterone. We observed that tumors grew less aggressively in male mice at three weeks post-surgical castration, a timepoint where testosterone levels were found to be the same as those in female mice (FIG. 5B). Principal component analyses (PCA) of spectral flow cytometry performed on CD8⁺ TILs in sham male, castrated male and sham female mice demonstrated that CD8⁺ T cells were distinct across groups, with the castrated male mice having an intermediate phenotype between those from sham male and female mice (FIG. 5C). Importantly, castration significantly decreased the proportion of CD8⁺ PE T cells (FIG. 5D). Further, we confirmed that enhanced tumor control in castrated mice was entirely dependent on intact CD8⁺ T cell immunity (FIG. 5E).

Second, we crossed E8i-Cre mice with Ar floxed mice to generate CD8⁺ T cell-specific AR KO mice. We confirmed that AR was expressed by CD8⁺ T cells in the WT mice (FIG. 5F). Loss of CD8⁺ T cell intrinsic AR signaling resulted in greater tumor protection, to a similar extent as found in females (FIG. 5G). Consistent with differences observed between male and female CD8⁺ TILs, the frequencies of CD8⁺ PE (FIG. 5H) and TCF1⁻ TOX⁺ exhausted (FIG. 5I) T cells were significantly reduced in the AR KO mice. Finally, in vivo delivery of enzalutamide, an AR inhibitor, resulted in a partial protection against tumor growth in male mice (FIG. 5J). Collectively, all three strategies to block AR signaling in vivo highlight the critical role of androgen in driving the PE phenotype of intratumoral CD8⁺ T cells and its consequence on tumor control.

### b) Androgen Signaling in CD8⁺ Progenitor Exhausted T Cells

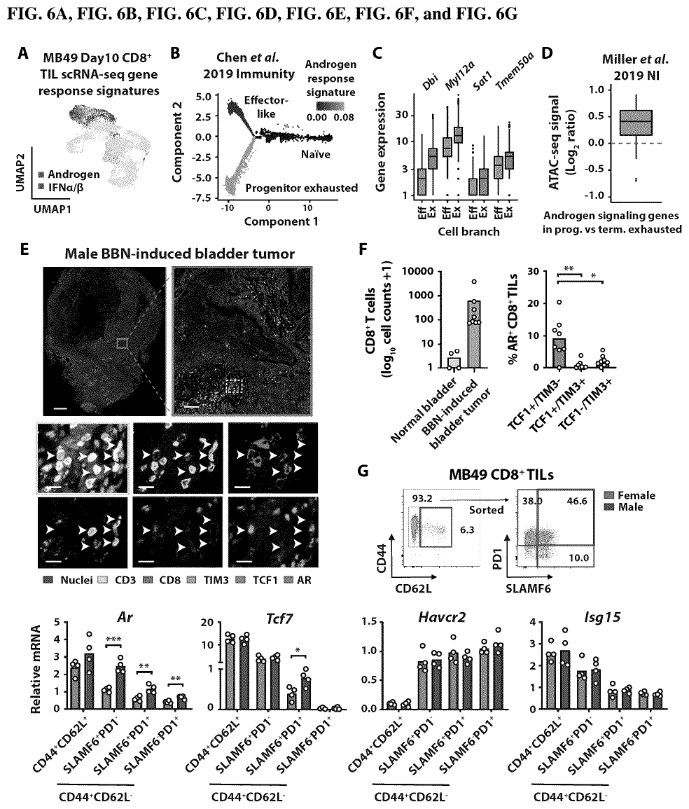

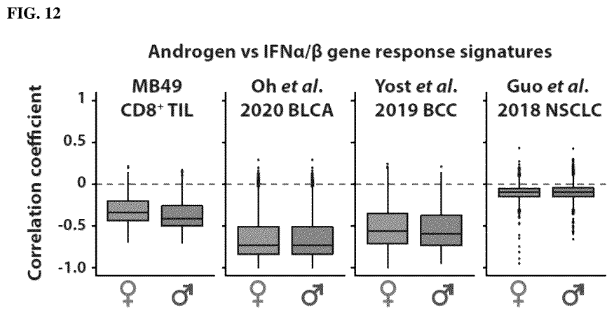

We so far demonstrated that there was a male-biased accumulation of PE CD8⁺ T cells that is driven by androgen and AR signaling. The next set of logical questions to address is if AR and its responsive genes are dynamically regulated during CD8⁺ T cell differentiation, and if so, whether AR and its target genes are preferentially expressed by PE cells. To this end, we analyzed CD8⁺ T cell transcriptomic data of our own as well as those that are publicly available. Upon analyzing scRNA-seq profiles of CD8⁺ TILs from MB49 (FIG. 6A), BBN-induced urothelial carcinoma (FIG. 12A) and lymphocytic choriomeningitis virus-specific CD8⁺ T cells (FIG. 6B), we found that the Hallmark Androgen Response—but not Hallmark Early or Late Estrogen Response—from Molecular Signatures Database (MSigDB) was uniquely enriched in clusters with expression profiles that reflect the Tcf7⁺ PE state (table 2). Dbi, Myl12a, Sat1 and Tmem50a, which all belong to the androgen responsive gene set, were expressed higher in the PE versus effector-like subset (FIG. 6C). Further, androgen responsive genes had greater chromatin accessibility in progenitor versus terminally exhausted T cells (FIG. 6D). Notably, we identified a negative correlation between CD8⁺ T cell intrinsic androgen and type I IFN gene signatures, the latter of which has been shown to repress TCF1 activity and inhibit the formation of CD8⁺ PE T cells, in MB49 tumors, as well as human bladder cancer (BLCA), basal cell carcinoma (BCC) and non-small cell lung cancer (NSCLC) (FIG. 12B). To address the mechanism underlying unique enrichment of androgen signaling in the PE subset, we investigated AR expression in CD8⁺ TILs in BBN-derived bladder tumors by multiplexed immunofluorescence imaging (FIG. 6E). We observed that AR was uniquely expressed in the TCF1⁺TIM3⁻ subset of tumor-infiltrating CD8⁺ T cells (FIG. 6F). Similarly, Tcf7 mRNA levels in CD8⁺ TILs coincided with SLAMF6 protein expression and showed a male bias in the CD44⁺CD62L⁻PD1⁺SLAMF6⁺ subset (FIG. 6G). We also observed that Ar mRNA levels decreased upon T cell activation, measuring highest in CD44⁺CD62L⁺ central memory cells and lowest in terminal exhausted CD44⁺CD62L⁻PD1⁺SLAMF6⁻ T cells (FIG. 6G). By comparison, Havcr2 mRNA (encoding TIM3) increased with T cell activation and exhaustion, while Isg15 (IFN responsive gene) decreased (FIG. 6G). Overall, our findings highlight the unique expression of AR and its signaling in CD8⁺ PE T cells.

TABLE 2
pvalue_hormone_IFN_by_sex. p values based on the enrichment of the
following signatures in 11 clusters of CD8⁺ T cells from Day 10 MB49 tumors:
1) HALLMARK ANDROGEN RESPONSE, 2) HALLMARK ESTROGEN RESPONSE EARLY, 3) HALLMARK
ESTROGEN RESPONSE LATE and 4) HALLMARK INTERFERON ALPHA RESPONSE.

| | | | | |
|---|---|---|---|---|
| | p-value for | p-value for | p-value for | p-value for |
| Cell | HALLMARK_ANDROGEN_{—} | HALLMARK_ESTROGEN_{—} | HALLMARK_ESTROGEN_{—} | HALLMARK_INTERFERON_{—} |
| cluster | RESPONSE | RESPONSE_EARLY | RESPONSE_LATE | ALPHA_RESPONSE |

Female

| | | | | |
|---|---|---|---|---|
| 0 | 0.545438468 | 0.969085451 | 0.761638309 | 0.015557228 |
| 1 | 0.170952499 | 0.975374487 | 0.839105968 | 0.025877216 |
| 2 | 0.021266644 | 0.77478547 | 0.28667719 | 0.195872674 |
| 3 | 0.823369609 | 0.999309548 | 0.539948252 | 0.48568425 |
| 4 | 0.237765851 | 0.657121199 | 0.226901275 | 0.059834502 |
| 5 | 0.553549282 | 0.937660178 | 0.589666919 | 0.524897372 |
| 6 | 0.384998154 | 0.997906311 | 0.923827239 | 0.314750831 |
| 7 | 0.023897882 | 0.663105205 | 0.448856565 | 0.073932699 |
| 8 | 0.191708779 | 0.930564415 | 0.335619605 | 0.072384308 |
| 9 | 0.326291389 | 0.840901464 | 0.55924134 | 2.12E−46 |
| 10 | 0.685399393 | 0.999999731 | 0.999991243 | 0.008273032 |

Male

| | | | | |
|---|---|---|---|---|
| 0 | 0.299458413 | 0.816242601 | 0.677769557 | 0.007097581 |
| 1 | 0.842356611 | 0.936535186 | 0.840593984 | 0.078249543 |
| 2 | 0.061004595 | 0.941016738 | 0.186773123 | 0.178719136 |
| 3 | 0.793439459 | 0.997706864 | 0.302194819 | 0.318266429 |
| 4 | 0.352258337 | 0.712401133 | 0.608288628 | 0.005521524 |
| 5 | 0.611769383 | 0.99136692 | 0.782977769 | 0.061909643 |
| 6 | 0.419718426 | 0.993967261 | 0.738993417 | 0.014739127 |
| 7 | 0.214339608 | 0.82253664 | 0.618007841 | 0.101427748 |
| 8 | 0.329369118 | 0.666778129 | 0.268550736 | 0.001482757 |
| 9 | 0.376289205 | 0.948656565 | 0.948656565 | 7.42E−45 |
| 10 | 0.967946642 | 0.99999921 | 0.999968972 | 0.197561899 |

### (1) Androgen Modulation of TCF1/Tcf7 Centered Transcriptional Network

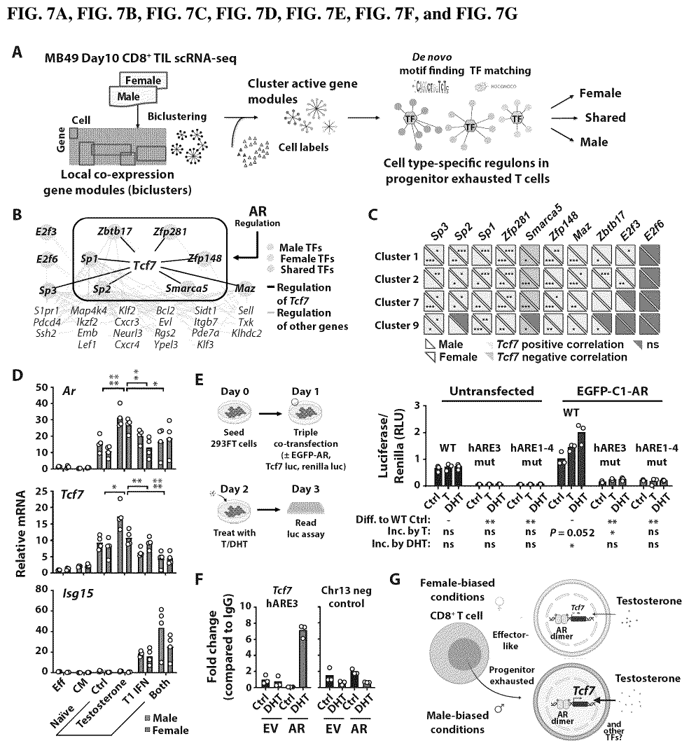

Given that a lack of androgen or AR in CD8⁺ T cells leads to a decreased proportion of CD8⁺ PE T cells, we hypothesized that AR regulates Tcf7 and the associated T cell exhaustion program. We applied IRIS3, a server we developed for cell-type-specific regulon inference from scRNA-seq data, to identify sex-specific transcriptional regulators of the Tcf7 gene module in CD8⁺ PE TILs using our scRNAseq data (FIG. 7A). Among 10 identified transcription factors, Smarca5, Zbtb17, Zfp281, Zfp148 and Maz were uniquely seen in males, E2f6 and E2f3 in females, and Sp1, Sp2 and Sp3 in both sexes (FIG. 7B). All but Smarca5 (negative) and E2f6 (not significant) showed a positive correlation with Tcf7 expression across various stages of CD8⁺ PE TIL differentiation (FIG. 7C). Notably, all male-biased or shared transcription factors except Sp3 and Maz have confirmed androgen response elements (AREs) by chromatin immunoprecipitation (ChIP) (FIG. 7B). How exactly these transcription factors mediate the immunological basis of sex bias warrant further investigation.

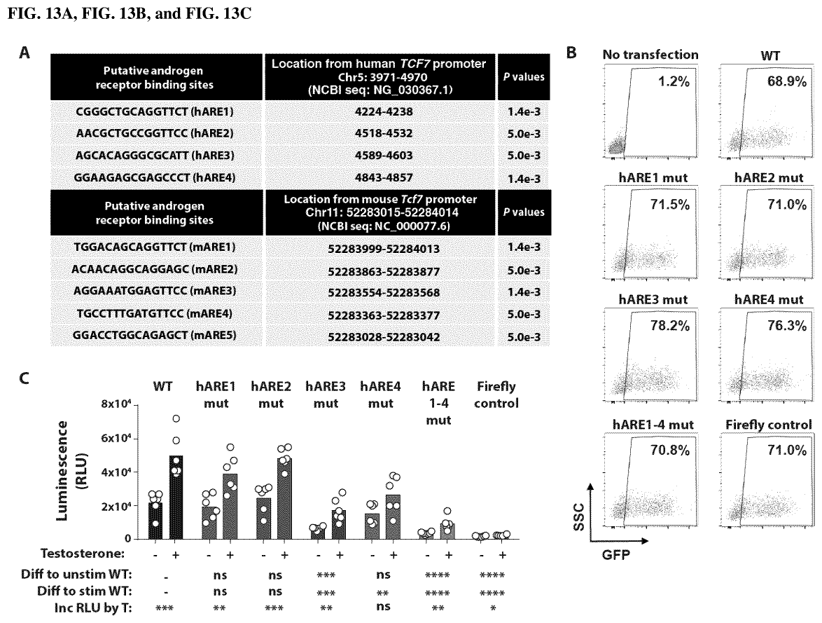

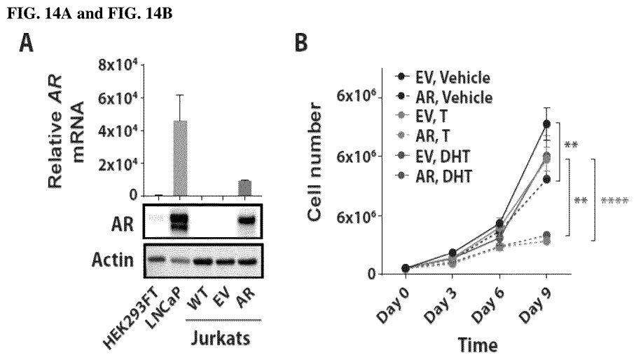

In agreement with IRIS3 findings, acute testosterone exposure significantly increased Tcf7 in male CD8⁺ T cells, which was in turn blunted by type I IFN (FIG. 7D). The effect of testosterone between sexes was not secondary to a lack of androgen signaling in female cells given the induction of Ar expression (FIG. 7D). Interestingly, AR motif scanning identified 4 and 5 putative AREs within one kilobase of the promoter immediately upstream of the human and mouse Tcf7 transcriptional start sites, respectively (FIG. 13A). We utilized a luciferase reporter assay to address if AR could directly regulate human Tcf7 transcription. Here, HEK293FT cells were co-transfected with an AR-expression vector and luciferase reporter plasmids whose expression was driven entirely by one kilobase WT or mutant Tcf7 promoter sequences lacking the putative AREs (FIG. 7E). We observed that testosterone and DHT stimulated Tcf7 promoter activity in an AR dependent manner (FIG. 7E). WT, but not promoter regions lacking either hARE3 or all AREs (hARE1-4), showed transcriptional activity in response to androgen (FIG. 7E, FIGS. 13B and 13C). This conclusion was substantiated using a stable AR-overexpressing Jurkat cell line, a system frequently used to uncover new T cell biology (FIG. 14A). AR-overexpressing Jurkat cells showed decreased proliferation in response to prolonged testosterone or DHT stimulation, consistent with its proposed role in driving exhaustion (FIG. 14B). Further, we performed ChIP-qPCR and found a near seven-fold increase in AR binding to the Tcf7 promoter at the hARE3 site compared to control only in the presence of androgen (FIG. 7F). Collectively, our findings highlight AR as a novel T cell intrinsic regulator of CD8⁺ T cell exhaustion, and it does so by transactivating Tcf7 and a sex-specific Tcf7-centered regulon in CD8⁺ PE T cells (FIG. 7G).

### C) DISCUSSION

Despite a clearly documented male bias in the incidence and mortality of various types of cancer, underlying biological mechanisms remain incompletely understood. The sporadic mechanistic studies on sexual dimorphisms in cancer have been largely focused on cancer genetics, epigenetics, and innate immunity. Whether there is a fundamental difference between male and female in the adaptive arm of immune surveillance such as CD8⁺ T cells is largely unknown. Here, we have gained new insights into the roles of AR in regulating CD8⁺ T cells in the tumor microenvironment. We have done so using multiple in vivo genetic models including the sex-reversed Four Core Genotype mice and CD8⁺ T cell-specific Ar KO mice, coupled with single cell transcriptome analysis of tumor-infiltrating CD8⁺ T cells, high dimensional flow cytometry and other assays. Our work highlights critical sex differences in CD8⁺ T cell-mediated anti-tumor immunity. We show that AR directly regulates Tcf7, a critical transcription factor involved in the early fate decisions of activated CD8⁺ T cells, and its signaling mediates a sex-biased formation of TCF1⁺CD8⁺ PE subset and control of tumor growth. Given the increasing recognition of immune evasion in oncogenesis and the central roles of CD8⁺ T cells in mediating anti-cancer immunity, our findings raise the possibility that AR-mediated predisposition for CD8⁺ T cell exhaustion interferes with the elimination of nascent immunogenic malignant cells and contributes to a male bias in both cancer incidence and mortality.

PD1^{Low}TCF1⁺CD8⁺ PE cells in this study lack expression of TIM3 and other checkpoint receptors, which have been previously referred to as a “memory-precursor-like” subset. They are presumably at an earlier phase of differentiation compared to CD8⁺ PE TILs with greater checkpoint receptor expression, but are not naïve given their high Cd44 and Cd69 expression. Varying levels of PD1 expression in CD8⁺ PE T cells can be a consequence of different experimental tumor models and inconsistent timings of sample collections and analyses. The PD1^{Low}TCF1⁺CD8⁺ PE subset contains tumor-antigen-specificity with the capacity to sustain long-lasting immunity, especially in response to immune checkpoint blockade. Their male-biased frequency is consistent with the notion that men benefit more from immune checkpoint blockade.

Androgens have been implicated in the development of bladder and other types of malignancies. While androgen is considered immunosuppressive for its inhibition of T cell thymic development, tissue-specific infiltration and effector differentiation, its impact on intratumoral CD8⁺ T cell differentiation and exhaustion has not been previously uncovered. We show for the first time that androgen signaling directly regulates Tcf7 and steers CD8⁺ TILs to exhaustion in a T cell-intrinsic fashion. It is striking that loss of AR in CD8⁺ T cells alone rendered male mice equally protective against cancer to female mice. Interestingly, we observed that type I IFN signaling is enriched in the PE subset and antagonizes androgen activity in regulating Tcf7. In light of a well-established female bias in the IFN response at baseline and upon active inflammation, a sex-specific balance of androgen and type I IFN signaling can underlie sex dimorphism in cancer immunity. Regulation of AR signaling, its molecular targets, and its interaction with type I IFN in CD8⁺ TILs warrant further investigation.

### D) MATERIALS AND METHODS

### (1) Study Design

The aim of this study was to uncover the role of CD8⁺ T cells in mediating sex differences in tumor progression and highlight a pertinent contribution of androgen receptor (AR) signaling in a CD8⁺ T cell differentiation program within the tumor microenvironment. To this end, we utilized a multi-omics approach to investigate CD8⁺ T cell immunity in several preclinical tumor models; and used surgical, genetic and pharmacological perturbation of AR signaling. All experiments used randomly assigned mice without investigator blinding. All data points and n values reflect biological replicates, unless indicated otherwise in scRNA-seq analyses. Raw data are available in table 4.

TABLE 4
Raw data

| aPD-1 | | | | | | | ADT | | | | | | ADT + aPD1 | | | | | |
|---|---|---|---|---|---|---|---|---|---|---|---|---|---|---|---|---|---|---|
| 0 | 0 | 0 | 0 | 0 | 0 | 0 | 0 | 0 | 0 | 0 | 0 | 0 | 0 | 0 | 0 | 0 | 0 | 0 |
| 32.2 | 26 | 29.2 | 26.2 | 35 | 28.4 | 34.7 | 31.5 | 26 | 34.2 | 37.4 | 26 | 27 | 23.9 | 24.4 | 30.1 | 34.2 | 28.6 | 25.2 |
| 44.5 | 51.8 | 37.3 | 48.4 | 52.3 | 52.9 | 42.5 | 47.9 | 42.2 | 48.1 | 52.9 | 31.3 | 40.9 | 36.5 | 36.9 | 38.2 | 44.8 | 44.1 | 42.9 |
| 51.4 | 69.7 | 44 | 72 | 56.8 | 76.8 | 53.3 | 44 | 44.9 | 61.6 | 54.2 | 32.4 | 46.1 | 46.1 | 49 | 41 | 50.9 | 50.3 | 50.6 |
| 63.8 | 74 | 56.7 | 78.3 | 60.2 | 79.5 | 49.2 | 84.7 | 45.5 | 68 | 63 | 46.2 | 62.3 | 48.2 | 46.9 | 42.8 | 54.7 | 51.1 | 50.7 |
| 67.5 | 70.6 | 49.7 | 68.7 | 71.3 | 63.8 | 48.6 | 106 | 33.1 | 79.3 | 71.6 | 44.1 | 73.1 | 47.4 | 47.6 | 47.5 | 52.4 | 45.3 | 48.3 |
| 54.4 | 66.3 | 58.9 | 44.8 | 63.6 | 53.3 | 44.2 | 114 | 13.7 | 82.7 | 75.5 | 57.3 | 79.2 | 43.9 | 43.6 | 50.3 | 50.4 | 34.8 | 32.5 |

### (2) Mice

C57BL/6 (WT; Stock number 000664), C57BL/6 Rag2^{-/-} (008449), Tcrb/Tcrd^{-/-} (002122), Ighm^{-/-} (002288), FCG (Four Core Genotype; 010905) and E8I-Cre (008766) mice were obtained from Jackson Labs. Ar^{flox/flox} mice were a gift from the laboratory of Dr. Xue Sean Li from Cedars-Sinai and were bred with the E8I-Cre mice to knockout AR within CD8⁺ T cells. FCG model involves manipulation of the Sry gene to create the following four “core” genotypes that can be used to investigate the contribution of sex chromosome complement and gonadal hormones to a given phenotype: XX (“XXF”), XY⁻ (“XYF”), XXSry (“XXM”) and XY⁻Sry (“XYM”). 5-12 weeks old mice, maintained in a specific pathogen-free environment, were used for experiments. Experiments—except for those utilizing FCG mice (Boston Children's Hospital)—were conducted under protocols approved by the Institutional Animal Care and Use Committee at the Medical University of South Carolina and the Ohio State University.

### (3) Tumor Model

To induce bladder carcinogenesis, male and female WT and Tcrb/Tcrd knockout or FCG C57BL/6 mice were fed ad libitum with 0.1% BBN (TCI America) water for 14 weeks and then switched to normal water. All mice were monitored daily for morbidity (i.e. palpable tumor/abdominal swelling, hunched posture and urine staining around perineum). If mice survived the 40-weeks-long regimen, they were considered as censored from the Kaplan-Meier survival curve analysis. BKL171 was derived from the bladder tumor of an XXM FCG mouse at the end of a 40 weeks-long BBN regimen.

MB49 (a gift from C. Voelkel-Johnson from the Medical University of South Carolina) and BKL171 mouse urothelial carcinoma cells were cultured in Dulbecco's modified Eagle's medium with 10% heat-inactivated fetal bovine serum and 1% penicillin/streptomycin. 5×10⁵ tumor cells were resuspended in 100 μL ice cold PBS for subcutaneous injection into the right flank of a mouse. For antibody-mediated T cell depletion experiments, mice were injected intraperitoneally with 200 μg of anti-mouse CD4 (Clone GK1.5, BioXCell) and/or CD8 neutralizing antibodies (Clone 53-6.7, BioXCell), followed by 100 μg thereafter on the indicated days. For experiments involving enzalutamide, mice were treated daily with either vehicle control or enzalutamide (25 mg/kg) in 1% carboxymethylcellulose with 0.1% Tween 80 and 2.5% DMSO through oral gavage starting on Day 5 post MB49 subcutaneous injection. Tumor surface area (width×length mm²) was measured using an electronic caliper starting on Day 4 post implantation.

### (4) Castration

To evaluate the contribution of androgen levels to tumor cell growth, male mice underwent surgical castration. Conditions described below, with the exception of gonadal removal, were also applied to both male and female mice to serve as sham controls. Briefly, aseptic techniques were used for all surgeries and mice were maintained under a sterile field. Mice were anesthetized with isoflurane, placed on a heating pad, given an ophthalmic lubricant to prevent corneal drying, and administered standard formula buprenorphine. Pain relief was also provided through use of Motrin supplemented water for 72 hours. Two to three mm bilateral incisions were made in the scrotum and the dartos fascia dissected to reveal the testes. After mobilization of the testes from the scrotum, the spermatic cord was cauterized and the scrotum closed using surgical tissue glue (Vetbond tissue adhesive). Mice were monitored daily for 3 days post-op and 2-3 times a week thereafter. Three weeks after surgery, mice were subcutaneously injected with the MB49 cell line as described above.

### (5) Testosterone Levels

Circulating free testosterone levels were analyzed in FCG and C57BL/6 mice at 7 and 10 weeks old, respectively. For FCG mice, blood was collected via submandibular vein using 1.5 mL eppendorf tubes coated with EDTA. Plasma testosterone concentration was determined using a testosterone ELISA kit from Enzo Life Sciences (ADI-901-065) according to the manufacturer's recommendations. Blood was also collected via submandibular vein for C57BL/6 mice. Here, one mL K3EDTA-coated MiniCollect (Greiner Bio-one, 450474) tubes were used to collect the blood and an ELISA kit from R&D Systems (KGE010) was used to measure testosterone levels according to manufacturer's recommendations.

### (6) Multispectral Immunofluorescence

At the terminal timepoint for male BBN-induced bladder cancer specimens described in the ‘Tumor models’ section, tumor-containing bladders were removed, alongside untreated normal bladder, fixed in 10% neutral buffered formalin (NBF, ThermoScientific, 5701) for 24 hours, transferred to 70% ethanol, and submitted to the Comparative Pathology and Mouse Phenotyping core at OSU for paraffin embedding. For multispectral immunofluorescence staining, four-to-five micron sections were deparaffinized with xylene and rehydrated with graded alcohol incubations. Pressurized antigen retrieval (Leica, AR9640) was performed at 125° C. for 1 min (Cuisinart, 574532315), followed by depressurization for 20 min and an additional 30 min for cooling. Slides were then incubated in a series of permeabilization and blocking steps that consisted of 0.1% Tween 20 (RPI, P20370) in 1×TBS (BioRad, 1706435) for 20 min, 0.3% Triton X100 (RPI, 111036) and 5% goat serum (Millipore, S26-LITER) in 1× TBS for 60 min, and 3% hydrogen peroxide (H₂O₂, Fisher, H325-100) for 8 min. Primary antibodies were incubated in an antibody diluent/block (Akoya, ARD1001EA) for 60 min in the following order, TIM3 (Cell Signaling, 83882) at 1:500 followed by Opa1620 (Akoya, FP1495001KT) at 1:200 for 10 min, CD8 (Cell Signaling, 98941) at 1:500 followed by Opa1480 (Akoya, FP1500001KT) at 1:250 for 6 min, AR (Abcam, ab108341) at 1:200 followed by Opa1570 (Akoya, FP1488001KT) at 1:200 for 10 min, TCF1 (Cell Signaling, 2203) at 1:500 followed by Opa1690 (Akoya, FP1497001KT) at 1:200 for 10 min, and CD3 (Epredia, RM-9107) at 1:500 followed by Opa1520 (Akoya, FP1487001KT) at 1:250 for 6 min. Anti-rabbit HRP-conjugated secondary antibodies (Leica, PV6119) were used to conjugated the fluorophores to the tissue. Fluorophores were incubated on each slide using an amplification buffer (Akoya, FP1498) for the indicated times and concentrations. Use of the pressurized antigen retrieval method described above, as well as an incubation step with 3% H₂O₂ for 5 min, was used to strip antibodies and quench any remaining peroxidase activity, respectively, between each set of primary, secondary, and fluorophore incubations. An unstained slide was included to detect autofluorescence of each fluorophore, and a set of six slides were used as drop out controls to ensure proper stripping occurred between each fluorophore conjugation. Finally, spectral dapi (Akoya, SKU FP1490) was applied according to manufacturer's recommendations and slides cover slipped using SlowFade Gold antifade mounting media (Invitrogen, S36937).

The multispectral images were captured at 20× magnification using Vectra Polaris imaging system. Briefly, an imaging protocol for each fluorophore was created to obtain best signal below the saturation limit for whole slide scanning. In addition, the autofluorescence signal was captured for each fluorophore. This imaging protocol was used to scan all slides in the study. Images were normalized and signal-to-noise ratios were tested for each fluorophore. Whole tissue section images were annotated and imported into the inForm software (Akoya, V2.5) for further analyses. First, images were annotated for biomarkers and fluorophores. The autofluorescence signal was isolated and the multiplexed fluorescence signals were unmixed. The inForm software allows development of machine learning-based segmentation of tissues categories and segmentation of cells. Unmixed image sections were sampled to make the training set for image processing and phenotyping algorithms. This training set was used to develop a pipeline for segmenting cells and phenotyping each biomarker. The algorithms were applied to all images for batch analyses. The resulting data was further analyzed using the phenoptr package (Akoya, V0.3.1) and R (V4.1.2)-programming to identify and quantify marker combination as shown in FIGS. 6, E and F.

(7) BBN-Induced Bladder Carcinogenesis and scRNAseq Studies

Similar to the description on BBN-induced bladder carcinogenesis in the ‘Tumor model’ methods section, C57BL6 female mice (Jackson Labs) were exposed to 0.05% BBN ad libitum in ambient drinking water. Control mice were exposed to 5% dextrose (BBN vehicle) containing water. Subjects were euthanized and bladders were harvested at pre-determined time points (10, 17, and 23 weeks). Whole bladders were mechanically and enzymatically digested [type IV collagenase (Sigma) for 60 min followed by GentleMACS dissociation (Miltenyi)]. Cells were strained through a 70 μM filter. Centrifuged pellets were treated once with ACK red blood cell lysis buffer (Thermo-Fisher), and non-viable cellular debris was removed with a dead cell removal kit (Miltenyi). Viable single cells were barcoded using the 10× Genomics Chromium Next platform (5′ Library and Gel Bead Kit, G chip) followed cDNA library generation and RNA sequencing (NovaSeq) at the Institute for Genomic Medicine located at Nationwide Children's Hospital in Columbus, Ohio.

All data analyses were conducted using R 4.1.1. Seurat (V4.0) was used for integrating individual samples, normalizing raw RNA read count matrices, selecting variable genes, and reducing dimensions using PCA. For visualization purposes, we implemented the Uniform Manifold Approximation and Projection (UMAP) algorithm using the top 50 principal component embeddings as input. We then used Seurat to construct a cell-cell similarity network and implemented the Louvain graph clustering algorithm to identify sub-populations of highly similar cells. To annotate sub-populations with cell types, we used SingleR (V1.8.0), which maps genes expression of each cell-to-cell type gene expression profiles curated by the Immunological Genome Project. Cd8a positive cells were extracted from “T cell” and “NKT” cell clusters and analyzed further as Cd8a⁺ TILs. After re-clustering on Cd8⁺ TILs, five clusters were identified, which were then annotated based on the expressions of major Cd8⁺ T cell marker including Cd44, Sell, I17r, Tcf7, Slamf6, Gzmb, Nkg7, Havcr2, Lag3, Tigit, Icos, Ctla4, Id2, Nr4a2, Nr4a3. Gene signature enrichment analysis (GSEA) was performed using the hypergeometric test to assess the enrichment of androgen response signature using the “HALLMARK_ANDROGEN_RESPONSE” gene set in the mSigDB database, which generated p-values for each cluster. A −log₁₀ transformation of the hypergeometric p-value was used for the color distribution of the UMAP.

### (8) Flow Cytometry

Mouse spleens and tumors were mechanically disrupted, with the latter additionally subjected to digestion with 1 mg/mL Collagenase D (Roche) for 30 min at 37° C. while shaken at 125 rpm. Excess volume of ice-cold PBS with 2% bovine serum albumin was used to inactivate the enzymatic activity. Red Blood Cell Lysis Buffer (BioLegend) was utilized on tissues before they were passed through 70 μm filters to prepare single cell suspensions. For cytokine production experiments, cells were re-stimulated by 50 ng/mL PMA (Sigma), 1 μg/mL Ionomycin (Sigma) and 1× Brefeldin A (BioLegend) in a 48-well plate for 2 hours at 37° C. For in vitro cultures, purified CD8⁺ T cells were left untreated or stimulated with 50 ng/mL testosterone (Sigma) and/or 50 U/mL mouse IFN alpha A (PBL Assay Science). Cells were stained at 4° C. with eFluor506 fixable viability dye for 10 minutes (Invitrogen), followed by extracellular surface markers and FcR block concurrently for 30 minutes. All intracellular staining was performed using the Foxp3 transcription factor staining kit (Invitrogen) according to the manufacturer's instructions. All samples were acquired on LSRFortessa or Cytek Aurora (high dimensional flow cytometry). All fluorochrome-conjugated antibodies are listed in table 3, in addition to the following cytokines TNFα (MP6-XT22) and IFNγ (XMG1.2).

TABLE 3
T cell exhaustion panel. List of antibodies for spectral flow cytometry.

| | | | |
|---|---|---|---|
| Target | Fluorophore | Company | Catalog number |

Extracellular T cell exhaustion panel markers

| | | | | |
|---|---|---|---|---|
| | Viability | Live dead blue | Invitrogen | L34962 |
| | dye | | | |
| Lineage | CD45 | BV510 | Biolegend | 103138 |
| markers | CD3 | BUV737 | BD Biosciences | 612771 |
| | CD8 | BUV496 | BD Biosciences | 750024 |
| | CD4 | APC-Fire 810 | Biolegend | 100480 |
| | CD11b | Alexa Fluor 532 | eBioscience | 58-0112-82 |
| | NK1.1 | BV570 | Biolegend | 108733 |
| T | PD1 | FITC | eBioscience | 11-9985-82 |
| cell | TIM3 | BV711 | Biolegend | 119727 |
| phenotypic | SLAMF6 | APC | eBioscience | 17-1508-82 |
| markers | CD44 | BUV661 | BD Biosciences | 741471 |
| | CD62L | BV421 | Biolegend | 104436 |
| | LAG3 | BUV805 | BD Biosciences | 748540 |
| | KLRG1 | Pacific Orange | eBioscience | 79-5893-82 |
| | VISTA | Super Bright 600 | eBioscience | 63-1083-82 |
| | TIGIT | BV650 | BD Biosciences | 744213 |
| | CX3CR1 | APC-Cy7 | Biolegend | 149040 |
| | ICOS | Super Bright 436 | eBioscience | 62-9949-82 |
| | CD27 | BUV563 | BD Biosciences | 741275 |
| | CD38 | BV750 | BD Biosciences | 747103 |
| | CD95 | BV480 | BD Biosciences | 746755 |
| | CD25 | BB515 | BD Biosciences | 564424 |
| | GITR | BUV615 | BD Biosciences | 751532 |
| | CD69 | PE-Cy5 | Biolegend | 104510 |

Intracellular T cell exhaustion panel markers

| | | | | |
|---|---|---|---|---|
| T | FOXP3 | eFluor 450 | eBioscience | 48-5773-82 |
| cell | TOX | PE | Miltenyi | 130-120-716 |
| phenotypic | TCF1 | PE-Cy7 | Cell Signaling | 90511s |
| markers | CTLA4 | PE-DAZZLE 594 | Biolegend | 106318 |
| | TBET | BV786 | BD Biosciences | 564141 |
| | KI67 | BUV395 | BD Biosciences | 564071 |
| T | BCL2 | Alexa Fluor 647 | Biolegend | 633510 |
| cell | EOMES | PerCP-eFluor 710 | eBioscience | 46-4875-82 |
| | GZMB | AF700 | Biolegend | 372222 |
| | CD107a | PerCP-Cy5.5 | Biolegend | 121626 |

Conventional flow cytometry data were analyzed on FlowJo. For spectral flow cytometry data, live CD45⁺CD11b⁻NK1.1⁻CD4⁻CD8⁺CD3⁺ singlets were gated using OMIQ and subsequently imported into R (V4.1.1) for all further analyses. To facilitate visualization of marker expression patterns, we mapped cells to a two-dimensional embedding using the UMAP algorithm. We then used PCA to visualize similarity among samples. We implemented the standard FlowSOM workflow to cluster cells based on all markers simultaneously using the R package FlowSOM (V2.1.24). The number of clusters used in the meta-clustering of cells was chosen based on elbow criterion. We utilized the UCell package (V1.1.0) to derive PE signature scores based on high (TCF1, SLAMF6, BCL2, CD44, CD69) and low (CD62L, PD1, LAG3, TIM3, CTLA4, TOX) expression.

### (9) Adoptive Transfer

CD8⁺ T cells were isolated by MACS (Miltenyi Biotec) from draining inguinal lymph nodes of MB49 bearing male and female mice 14 days post implantation (FIG. 2B). Alternatively, they were isolated by FACS from Day 12 MB49 TILs in male and female mice based on TIM3⁻SLAMF6⁺ surface expression (FIG. 4D). Donor cells were intravenously administered into immunodeficient mice (Tcrb/Tcrd KO in FIG. 2B; Rag2 KO in FIG. 4D). Recipient mice were injected with MB49 tumors at indicated time points and monitored for tumor growth.

### (10) Single Cell RNA Sequencing

6 weeks old WT male and female C57BL/6 mice were inoculated subcutaneously with 5×10⁵ MB49 tumor cells. On Day 10 post inoculation, single cell suspensions were prepared from the tumors after mechanical disruption and enzymatic digestion with 1 mg/mL Type IV Collagenase (Roche). Live tumor infiltrating CD8⁺ T cells (CD3⁺ CD8⁺ CD4⁻) were sorted on a BD FACSAria Ilu Cell Sorter and immediately processed for scRNA-seq. Experimental procedures for scRNA-seq followed established techniques using the Chromium Single Cell 3′ Library V3 Kit (10× Genomics). Briefly, FACS-sorted CD8⁺ T cells were loaded onto a 10× Genomics Chip A and emulsified with 3′ Single Cell GEM beads using a Chromium™ Controller. Libraries were constructed from the barcoded cDNAs (Translational Science Laboratory at the Medical University of South Carolina) and sequenced for approximately 300 million reads/sample on a NovaSeq S4 flow cell (Illumina) at the VANTAGE facility (Vanderbilt University Medical Center).

(11) scRNA-Seq Data Analysis

Using the Cell Ranger software, we converted BCL files into FASTQ files, trimmed adapters and primer sequences, mapped reads to the mm10 reference genome, and quantified expression levels. In this step, to eliminate low-quality and dying cells, we filtered out cells with counts less than 200 and those with >5% mitochondrial counts. Then, we used the Seurat software for the downstream analysis, based on the count data obtained from Cell Ranger. Specifically, we normalized counts using the LogNormalize approach, visualized cells in a low-dimensional space using the UMAP algorithm, and determined cell clusters using the Louvain graph clustering algorithm. This process resulted in identification of 11 cell clusters. Then, we identified cell type markers conserved between males and females for each cell cluster and also the genes that are differentially expressed (DE) between males and females using a Wilcoxon Rank Sum test, and adjusted DE p values for multiple testing using the Bonferroni correction. For the pseudotime analysis, we used the Slingshot software with the Seurat-processed data for the cell clusters 1, 2, 6, 7, 9, and 10. Gene set enrichment analyses were implemented using the hypergeometric test with the Kyoto Encyclopedia of Genes and Genomes (KEGG) gene sets obtained from the MSigDB. For the sex-biased gene expression heatmap, we first checked DE between males and females for each gene based on Bonferroni-adjusted p-values and visualized directions of sex bias based on corresponding log fold changes. For the relative cluster enrichment heatmap, we first checked whether each corresponds to a marker conserved between males and females based on Bonferroni-adjusted p-values, and visualized directions of enrichment based on corresponding log fold changes.

### (12) Secondary Analysis of Single-Cell RNA-Seq Data

We implemented secondary analyses of single cell RNA-seq data. We downloaded the count data from the GEO database with the accession number GSE99254 and selected only the cells corresponding to the CD8⁺ T cells from tumors. We also downloaded the count data for BCC from the GEO database with the accession number GSE123813, and we selected only the cells corresponding to CD8⁺ T cells and pre-treatment. Then, for both datasets, we used the Seurat analysis workflow described in “Single cell RNA-seq data analysis”. Additionally, we downloaded the count data from the GEO database with the accession number GSE131535. Then, we used the Monocle 2 analysis workflow described in “Single cell RNA-seq data analysis”. We also downloaded the count data from the GEO database with the accession number GSE149652 and further selected the cells corresponding to CD8⁺ T cells from Anti-PD-L1-treated human bladder cancer. Then, for each of males and females, we calculated Pearson correlation coefficients between genes annotated for “HALLMARK_ANDROGEN_RESPONSE” and those annotated for “HALLMARK_INTERFERON_ALPHA_RESPONSE” gene sets in the mSigDB database.

### (13) Secondary Analysis of Bulk ATAC-Seq Data

We implemented the secondary analysis of bulk ATAC-seq data. Specifically, we downloaded the peak calling results from the GEO database with the accession number GSE122713, where reads were mapped using Bowtie2 and peak calling was performed using MACS2. We annotated these peaks using HOMER and extracted the counts corresponding to promoter regions (annotated as “promoter-TSS”) of the genes annotated for the “HALLMARK_ANDROGEN_RESPONSE” gene set in the mSigDB database. Finally, we calculated the log 2-transformed ratios of Slamf6⁺Tim3⁻GP33 Tetramer*CD44⁺PD-1⁺ CD8⁺ T cells over Slamf6-Tim3⁺GP33 Tetramer*CD44⁺PD-1⁺ CD8⁺ T cells from LCMV-infected mice.

(14) RNA Isolation and qPCR Analysis

RNA was extracted from FACS/MACS-isolated CD8⁺ T cells and reverse-transcribed using RNeasy Micro Kit (Qiagen) and SuperScript™ IV VILO™ Master Mix with ezDNase™ Enzyme (ThermoFisher Scientific), respectively. Quantitative PCR was performed with the following primers:

Ar:
forward,
(SEQ ID NO: 1)
5′-TCCAAGACCTATCGAGGAGCG-3′
reverse,
(SEQ ID NO: 2)
5′-GTGGGCTTGAGGAGAACCAT-3′
Tcf7:
forward,
(SEQ ID NO: 3)
5′-CCACTCTACGAACATTTCAGCA-3′
reverse,
(SEQ ID NO: 4)
5′-ACTGGGCCAGCTCACAGTA-3′
Havcr2:
forward,
(SEQ ID NO: 5)
5′-TCAGGTCTTACCCTCAACTGTG-3′
reverse,
(SEQ ID NO: 6)
5′-GGCATTCTTACCAACCTCAAACA-3′
Isg15:
forward,
(SEQ ID NO: 7)
5′-GGTGTCCGTGACTAACTCCAT-3′
reverse,
(SEQ ID NO: 8)
5′-CTGTACCACTAGCATCACTGTG-3′
β-actin:
forward,
(SEQ ID NO: 9)
5′-AGCTGAGAGGGAAATCGTGC-3
reverse,
(SEQ ID NO: 10)
5′-TCCAGGGAGGAAGAGGATGC-3′
Sry (Set 1):
forward,
(SEQ ID NO: 11)
5′-TTGTCTAGAGAGCATGGAGGGCCATGTCAA-3′
reverse,
(SEQ ID NO: 12)
5′-CCACTCCTCTGTGACACTTTAGCCCTCCGA-3′
Sry (Set 2):
forward,
(SEQ ID NO: 13)
5′-TGGGACTGGTGACAATTGTC-3′
reverse,
(SEQ ID NO: 14)
5′-GAGTACAGGTGTGCAGCTCT-3′

### (15) De Novo AR Motif Prediction in Tcf7 Promoter

AR motifs were predicted from human (Chr 5: 3971-4970, NCBI seq: NG_030367.1) and mouse (Chr 11: 52283015-52284014, NCBI seq: NC_000077.6) Tcf7 promoter sequence of one kilobases (kb) upstream from its transcriptional start site. The following AREs, AGAACAnnnAGTACT (SEQ ID NO: 15) and AGAACAnnnAGTGCT (SEQ ID NO: 16), were scanned via Motif Alignment and Search Tool (MAST) at a positional p value of <0.005.

### (16) Luciferase Reporter Assays

WT or mutated human Tcf7 promoter sequence of one kb upstream from its transcriptional start site was cloned into the pMCS-Red Firefly Luciferase vector (Thermo Fisher). Mutants lacked individual (“Mut 1-4”) or all putative AR binding sites. Briefly, 40,000 HEK293FT cells were grown on a 96 well plate at 80% confluence. Cells were transfected with 200 ng of pEGFP-C1-human AR (a gift from Michael Mancini, Addgene plasmid #28235) and indicated Tcf7 luciferase reporter plasmids using Lipofectamine 2000 (Thermo Fisher) on Day 0 in triplicates. Renilla luciferase gene expression was used to ensure transfection efficiency (Thermo Fisher). Twenty four hours after transfection, cells were treated with 500 ng/ml testosterone or 50 nM DHT for another 24 hours. Cells were then harvested and luciferase activity was analyzed by using Dual-Luciferase Reporter Assay System (Promega).

### (17) Generation of AR-Expressing Jurkat Cells

AR lentiviral expression plasmid (a gift from Karl-Henning Kalland, Addgene #85128) was clonally selected alongside empty vector control (a gift from Jan Rehwinkel, Addgene #120848) and maxi-prepped (Qiagen) from bacterial streaked ampicillin containing agar plates (Invitrogen). Lentivirus was generated using HEK239FT cells and virus-containing media was filtered (Sigma, SE1M003M00) and concentrated (Takara, 631231) before being used to infect the Jurkat cell line (ATCC, TIB-152) in the presence of 1 μg/ml polybrene (Sigma). After 48 hours, infected cells were selected using 4 μg blasticidin (Gibco) for 10 days.

### (18) Western Analysis

All cells were lysed using RIPA (Thermo Fisher) buffer plus protease and phosphatase inhibitors (Thermo Fisher). Equal amounts of protein were separated on SDS polyacrylamide gels (BioRad) and transferred to PVDF membranes (Millipore). Antibodies used for immunoblot analyses were against AR (Abcam, ab108341) and P-actin (Abcam, ab8226). Secondary antibodies included anti-rabbit DyLight800 IgG (Cell Signaling, 5151) and anti-mouse DyLight680 IgG (Cell Signaling, 5470).

To collect enough protein lysate for immunoblot analyses, spleens from WT AR^{fl/(fl)} and E8iCre-AR^{fl/(fl)} mice were mechanically homogenized, incubated with a red cell lysis buffer (Biolend, 420302) and passed through 70 micron filters. CD8⁺ T cells were isolated using a mouse CD8 isolation kit (Stemcell, 19853) according to manufacturer's recommendations. Splenocytes were then stimulated using plate-bound 5 μg/mL CD3 (Biolegend, 100359) and 2 μg/mL CD28 (Biolegend, 102121) plus 40 ng/mL IL2 (NIH) for 72 hours. Afterwards, cells were washed and expanded in the presence of 40 ng/mL IL2 for another 72 hours before they were pelleted and lysed for immunoblot analyses.

### (19) Proliferation Assay

Empty vector and androgen receptor-expressing Jurkat cells were seeded into six well plates (Gibco) at 2×10⁵ cells per well and treated with 100 nM testosterone (Sigma) or DHT (Millipore), alongside methanol vehicle control (Thermo Fisher). Every three days, cells were counted using the BioRad TC20 automated cell counter, passaged, and re-stimulated with the same androgen concentrations over a time course of 3, 6, and 9 days.

### (20) ChIp-qPCR

Empty vector and androgen receptor-expressing Jurkat cells were treated with 100 nM testosterone, 100 nM DHT or methanol vehicle control for 24 hours before being subjected to chromatin immunoprecipitation (IP). Briefly, 1×10⁷ cells were fixed with formaldehyde, quenched with glycine, washed, and sonicated using the Covaris E220 Evolution sonicator (peak power: 140, duty factor: 5, cycles/burst: 200, average power: 7) for 16 min. Cleared nuclear extracts were incubated with either AR (Abcam, ab108341) or species-matched IgG (Cell Signaling, 3900) pre-coupled magnetic protein A beads (Thermo Fisher) and rotated overnight at 4° C. Beads were washed with low salt (0.1% SDS, 1% Triton X-100, 2 mM EDTA, 150 mM NaCl, 20 mM Tris-HCl, pH 8.0), high salt (0.1% SDS, 1% Triton X-100, 2 mM EDTA, 500 mM NaCl, 20 mM Tris-HCl, pH 8.0), LiCl (0.25 M LiCl, 1% NP-40, 1% DOC, 1 mM EDTA, 10 mM Tris-HCl, pH 8.0) and TE (Invitrogen) wash buffers before protein and DNA were eluted with freshly prepared sodium bicarbonate buffer (1% SDS, 100 mM NaHCO₃) at 55° C. Samples were then incubated with proteinase K (Thermo Fisher) overnight at 55° C. and DNA was purified using a PCR cleanup column (Qiagen) according to manufacturer's recommendations.

Quantitative PCR was performed using the IQ SYBR Green Supermix (BioRad) and amplification was conducted using the StepOnePlus Real-Time PCR System (Applied Biosystems) with the following primers: hDNase NC2-chr13: forward, 5′-GGCACATACCCATTTGTCCCAACA-3′ (SEQ ID NO: 17) reverse, 5′-GACAAAGGTGTCAAGAACACACAATGGG-3′ (SEQ ID NO: 18) hTcf7 hAR 3: forward, 5′-GTACGAGCACAGCCTCAAG-3′ (SEQ ID NO: 19) reverse, 5′-GCCGTGGTGTGAACTGTAT-3′ (SEQ ID NO: 20). Percent input DNA was normalized to the corresponding species-matched IgG controls.

### (21) Integrated Cell-Type-Specific Regulon Inference Server 3 (IRIS3) Analysis

In order to computationally identify transcription factors (TFs) that regulate Tcf7 expression in CD8⁺ TILs from male and female mice, we applied IRIS3, an integrated web server for cell-type-specific regulon (CTSR) prediction, on our scRNA-seq data. Briefly, IRIS3 workflow includes five steps: (i) cell cluster prediction, (ii) functional co-expressed gene module detection, (iii) cell cluster active gene module determination, (iv) de novo motif finding, and (v) TF matching and CTSR determination. We reasoned that CTSRs could build reliable constructions of global transcriptional regulatory networks encoded in a specific cell type and provide insight into underlying regulatory mechanisms. Specifically, the male and female datasets were analyzed separately via IRIS3, with the “bicluster overlap rate” parameter at 0.6 and all other parameters as default. We directly used the cell cluster labels determined in the previous step to ensure the consistency. For each predicted CTSR, we considered all significantly matched TFs in the motif comparison result (using TOMTOM and HOCOMOCO database as regulators, rather than only the top TF described in the IRIS3 tutorial. For the progenitor T cell cluster, genes among CTSRs were then merged based on the corresponding TF. Male-specific, female-specific and shared TFs were determined by comparing the corresponding TFs in male and female data. Gene regulatory networks were constructed to indicate the predicted TF-gene regulatory relations via Cytoscape.

### (22) Statistical Analysis

Tumor growth was analyzed by a repeated measures two-way ANOVA. Overall survival was visualized using Kaplan-Meier curves and analyzed using a log-rank test. Statistical significance of analyses pertinent to scRNA-seq data was determined using Wilcoxon rank-sum test. Primary method of statistical analysis for other outcomes was a two-sided independent-sample t-test. For all statistical testing, p values<0.05 were considered significant, and they were adjusted for multiple testing using Bonferroni correction as appropriate.

### 2. Example 2: Novel Mechanisms of Androgen Receptor-Centered Transcriptional Regulatory Network in Regulating CD8⁺ T Cell Exhaustion and Sex Bias in Cancer

Sex bias in cancers arising from nonreproductive organs is known but poorly understood. We recently reported on a T cell-intrinsic role of androgen receptor (AR) in driving CD8⁺ T cell exhaustion which underlies the poorer tumor control in males. This study aims to understand how AR regulates CD8⁺ tumor-infiltrating lymphocytes (TILs) by identifying its genome-wide targets. We created a CD8⁺ T cell-specific AR knockout (KO) mouse model by crossing E8I-cre mice with Ar-floxed mice, which we challenged with syngeneic bladder tumor MB49. We then monitored tumor growth and performed spectral flow cytometry using a T cell exhaustion panel. Further, we used Cleavage Under Targets and Tagmentation—sequencing (CUT&Tag-seq) to map the entire AR targets in CD8⁺ TILs. Loss of AR in CD8⁺ T cells significantly slowed the growth of MB49 in male but not female mice. CD8⁺ TILs from male CD8 AR KO mice showed reduced TOX+ TCF1⁻ terminally exhausted subset (69%; P<0.05) and TOX expression (72%; P=0.0583) compared to controls. Similarly, TOX expression decreased by 43% in AR-deleted CD8⁺ T cells following chronic TCR stimulation in vitro (P<0.0001). Finally, using CUT&Tag-seq, we found that AR binds directly to promoters of multiple key transcriptional regulators of T cell exhaustion, including Tcf7 and Tox.

### 3. Example 3: Novel Mechanisms of Androgen Receptor-Centered Transcriptional Networks in Regulating CD8⁺ T Cell Exhaustion

### a) Results

### (1) Ablation of Androgen Receptor (AR) Signaling Attenuates Tumor Aggressiveness in Murine Bladder Cancer

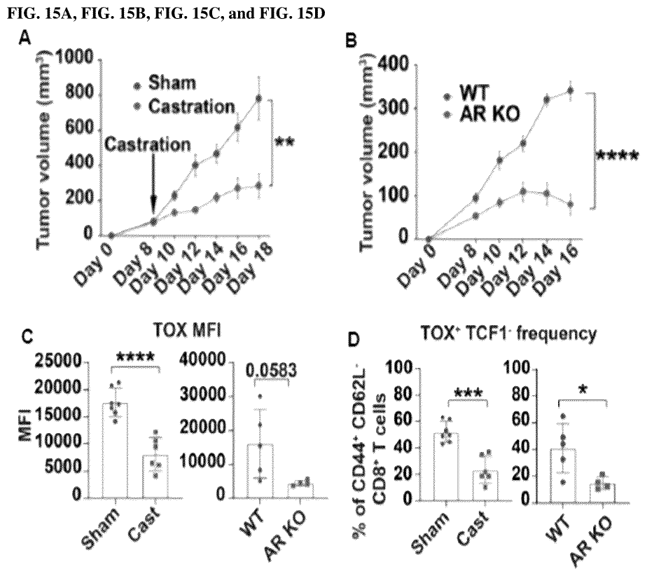

We explored the impact of androgen receptor (AR) signaling ablation on tumor aggressiveness in murine bladder cancer. Panel A (FIG. 15A) depicts MB49 tumor growth in male wild-type CD57B/6 mice subjected to surgical castration (red, n=6) or sham surgery (blue, n=7). In Panel B (FIG. 15B), we observe MB49 tumor growth in male AR^{floxed} (blue, WT, n=5) versus E8iCre−AR^{floxed} (red, AR KO, n=5) mice. Panel C (FIG. 15C) illustrates the mean fluorescence intensity (MFI) of TOX from tumor-infiltrating CD8+ T cells across the experiments in Panels A and B. Additionally, Panel D (FIG. 15D) demonstrates the frequency of TOX+TCF1− terminally exhausted tumor-infiltrating CD8+ T cells as a percentage of Cd44+CD62L−CD8+ T cells in the same experiments. These findings highlight the potential role of AR signaling in modulating tumor aggressiveness and suggest a possible avenue for therapeutic intervention.

(2) AR Maintains TCF1^{high} Stem-Like States and Represses Effector Differentiation and Function in Murine CD8⁺ T Cells, which is Reversed by AR Knockout

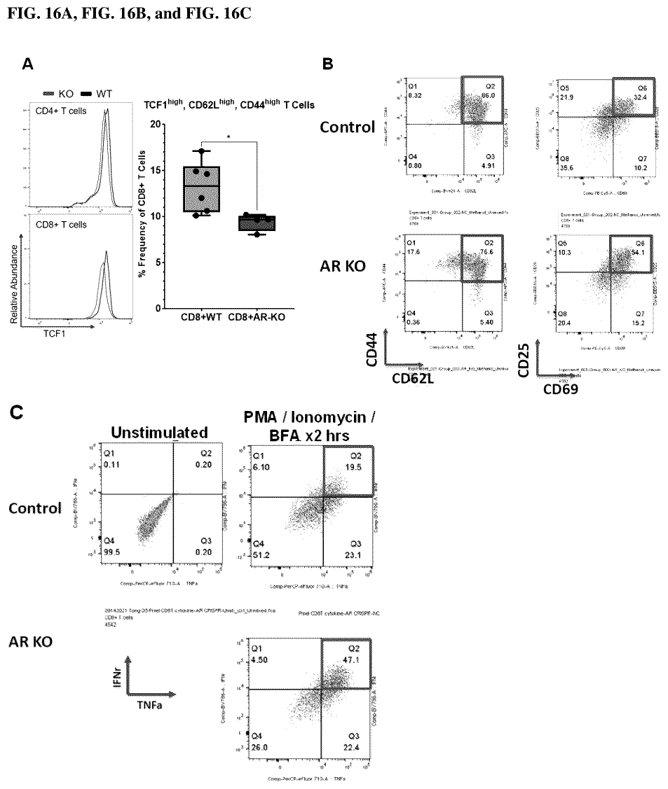

Further we investigate the role of androgen receptor (AR) in maintaining TCF1^{high} stem-like states and repressing effector differentiation and function in murine CD8+ T cells, with implications for tumor immunity (FIG. 16). Panel A (FIG. 16A) presents flow cytometry characterization of splenic CD8+ T cells from non-tumor-bearing wild-type (AR^{floxed}, grey, n=6) or CD8-specific AR knockout (E8iCre−AR^{floxed}, red, n=4) mice, revealing a lower frequency of TCF1high central memory cells in AR knockout CD8+ T cells. In Panel B, (FIG. 16B) CRISPR-mediated AR knockout in splenic CD8+ T cells from non-tumor-bearing Pmel mice, activated in vitro with gp100 peptide, leads to a lower frequency of CD44+CD62L+ central memory CD8+ T cells and a higher frequency of CD25+CD69+ effector-like cells compared to scrambled guide-RNA control conditions. Furthermore, Panel C (FIG. 16C) demonstrates that CD8⁺ T cells with CRISPR-mediated AR knockout exhibit higher IFNγ expression upon PMA/ionomycin+BFA stimulation. Statistical significance in Panel A is determined by Student's t test (*P≤0.05). These results highlight the pivotal role of AR in regulating CD8+ T cell differentiation and function, suggesting potential therapeutic strategies for enhancing anti-tumor immunity.

### (3) CUT&Tag Assay Identifies AR Genomic Binding Loci in Murine CD8⁺ TILs

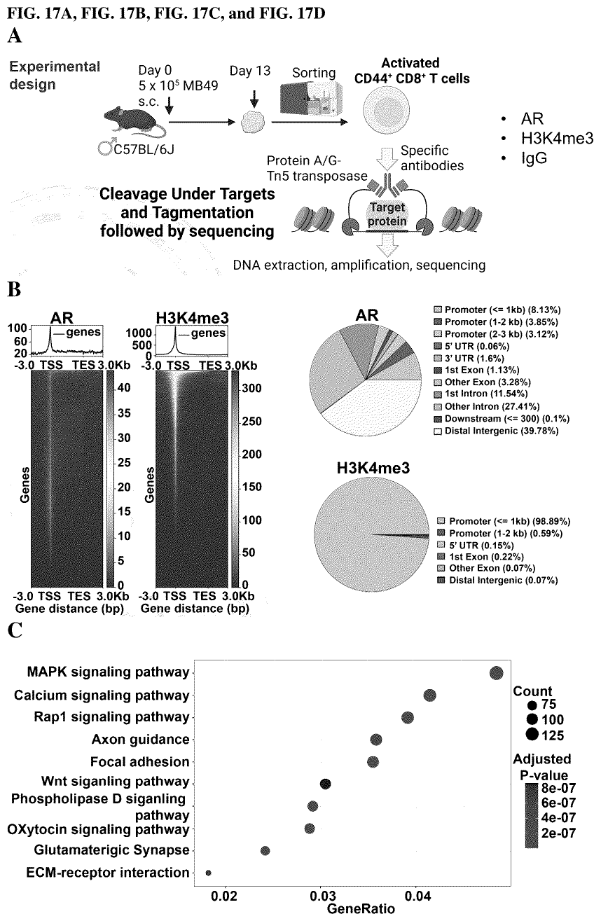

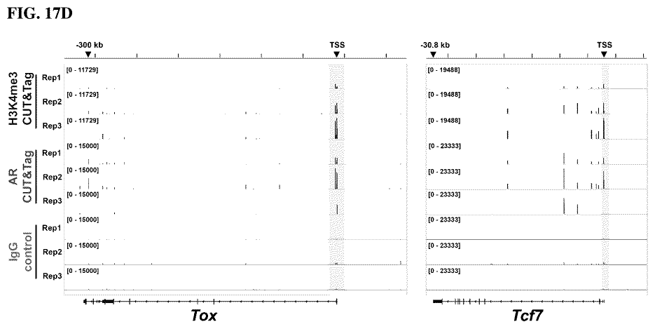

In the experiment detailed in FIG. 17, we employed the CUT&Tag assay to uncover and characterize androgen receptor (AR) genomic binding loci in murine CD8+ tumor-infiltrating lymphocytes (TILs) from the MB49 model. Panel A (FIG. 17A) illustrates the schematic of the CUT&Tag assay methodology, employed to pinpoint transcriptional targets of AR in these specific cells. Panel B (FIG. 17B) depicts the distances of AR and H3K4me3 binding sites from transcriptional start sites (TSS), shedding light on their regulatory potential. Furthermore, Panel C (FIG. 17C) presents the results of functional enrichment analysis of AR-regulated genes, showcasing the top 10 enriched gene ontology terms. Lastly, Panel D (FIG. 17D) offers gene browser tracks displaying AR, H3K4me3, and IgG control readouts across Tox and Tcf7 gene loci, providing insights into the genomic landscape of AR-mediated regulation. These findings provide valuable insights into the molecular mechanisms underlying AR-mediated regulation of CD8⁺ TILs in the tumor microenvironment.

(4) Androgen Deprivation Therapy Alters CD8⁺ T Cell Exhaustion Programs in Men with Castrate-Sensitive Prostate Cancer

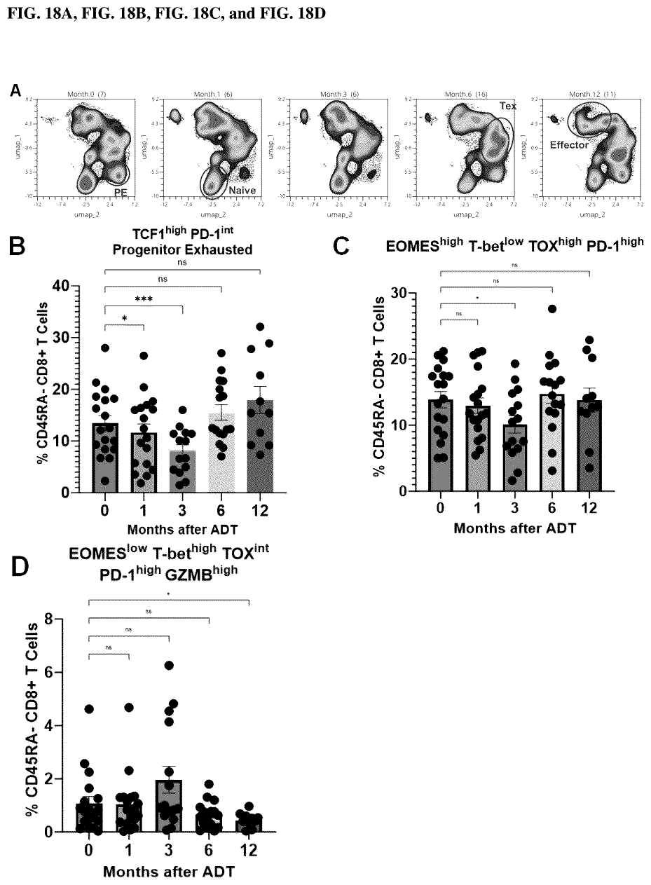

In our investigation detailed in FIG. 18, we explored the effects of androgen deprivation therapy (ADT) on CD8⁺ T cell exhaustion programs in men with castrate-sensitive prostate cancer. Panel A (FIG. 18A) showcases high-dimensional flow cytometry analysis using a 23-marker T cell exhaustion panel on peripheral blood mononuclear cells collected at month 0 (baseline) before ADT initiation or at 1, 3, 6, or 12 months post-initiation of ADT in 18 men with newly diagnosed localized or metastatic castrate-sensitive prostate cancer. The data are presented in uniform manifold approximation and projection (UMAP) space, with manual cluster annotations represented by red circles. Panels B, C, and D depict the frequencies of distinct CD8⁺ T cell exhaustion subsets over the course of ADT. Specifically, Panel B (FIG. 18B) shows the frequency of TCF1^{high} PD-1^{intermediate} progenitor exhausted cells, Panel C (FIG. 18C) presents the frequency of EOMES^{high} T-betlow TOXhigh PD-1^{high} terminal exhausted-like cells, and Panel D (FIG. 18D) illustrates the frequency of EOMES^{low} T-bet^{high} TOX^{intermediate} PD-1^{high} GZMB^{high} effector-like cells, all as percentages of CD45RA-CD8⁺ T cells. Statistical significance in Panels B-D was determined by a mixed effects model for repeated measures data, with error bars representing±SEM. Significance levels are denoted as *P≤0.05 and ***P≤0.001, with P values corrected for multiple hypothesis testing using the Dunnett method. These findings offer insights into the dynamic changes in CD8⁺ T cell exhaustion profiles induced by ADT in men with castrate-sensitive prostate cancer.

### (5) Androgen Receptor Blockade of In Vitro Expanded CD8+ Bladder Cancer TILs Promotes Effector Differentiation and Function

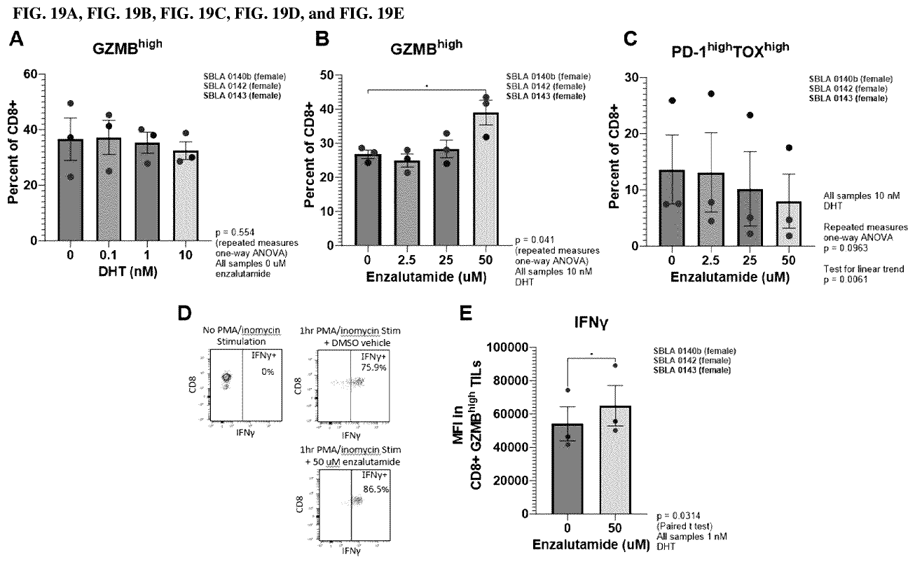

In FIG. 19, we investigated the impact of androgen receptor (AR) blockade on in vitro expanded CD8⁺ tumor-infiltrating lymphocytes (TILs) from female patients with bladder cancer, focusing on effector differentiation and function. Panel A (FIG. 19A) illustrates the effect of dihydrotestosterone (DHT) treatment on the CD8+GZMB^{high} population within ex vivo expanded TILs. Panel B (FIG. 19B) demonstrates the impact of AR blockade using 50 uM enzalutamide in the presence of 10 nM DHT on the frequency of CD8+GZMB^{high} TILs compared to vehicle control. Furthermore, Panel C (FIG. 19C) shows the frequency of PD-1^{high}TOX^{high} CD8+ TILs with varying doses of enzalutamide. Panel D (FIG. 19D) assesses IFNγ production in CD8+ TILs after PMA/ionomycin/golgi block stimulation with or without enzalutamide. Lastly, Panel E presents the median fluorescence intensity (MFI) of IFN-gamma within the CD8+GZMB^{high} population. Statistical significance in Panels A-C was determined by repeated measures ANOVA, with P values corrected for multiple hypothesis testing using the Dunnett method. In Panel E (FIG. 19E), statistical significance was determined by Student's paired t test (*P≤0.05). These results shed light on the regulatory role of AR blockade in promoting effector differentiation and function in CD8+ TILs from bladder cancer patients, offering therapeutic implications for AR-targeted interventions in bladder cancer immunotherapy.

### F. REFERENCES

A. Butler, P. Hoffman, P. Smibert, E. Papalexi, R. Satija, Integrating single-cell transcriptomic data across different conditions, technologies, and species. Nat Biotechnol 36, 411-420 (2018).
A. C. Roden et al., Augmentation of T cell levels and responses induced by androgen deprivation. J Immunol 173, 6098-6108 (2004).
A. C. Scott et al., TOX is a critical regulator of tumour-specific T cell differentiation. Nature 571, 270-274 (2019).
A. Clocchiatti, E. Cora, Y. Zhang, G. P. Dotto, Sexual dimorphism in cancer. Nat Rev Cancer 16, 330-339 (2016).
A. Dunford et al., Tumor-suppressor genes that escape from X-inactivation contribute to cancer sex bias. Nat Genet 49, 10-16 (2017).
A. G. Robertson et al., Comprehensive Molecular Characterization of Muscle-Invasive Bladder Cancer. Cell 171, 540-556 e525 (2017).
A. Ma et al., IRIS3: integrated cell-type-specific regulon inference server from single-cell RNA-Seq. Nucleic Acids Res 48, W275-W286 (2020).
A. P. Arnold, Mouse models for evaluating sex chromosome effects that cause sex differences in non-gonadal tissues. J Neuroendocrinol 21, 377-386 (2009).
A. Rao, S. D. Cramer, Identification of a polymorphism in the ARE 1 region of the PSA promoter. Proc. Am. Assoc. Cancer Res 40, (1999).
A. Subramanian et al., Gene set enrichment analysis: a knowledge-based approach for interpreting genome-wide expression profiles. Proc Natl Acad Sci USA 102, 15545-15550 (2005).
B. C. Miller et al., Subsets of exhausted CD8 (+) T cells differentially mediate tumor control and respond to checkpoint blockade. Nat Immunol 20, 326-336 (2019).
B. Langmead, S. L. Salzberg, Fast gapped-read alignment with Bowtie 2. Nat Methods 9, 357-359 (2012).
C. H. Li, S. Haider, Y. J. Shiah, K. Thai, P. C. Boutros, Sex Differences in Cancer Driver Genes and Biomarkers. Cancer Res 78, 5527-5537 (2018).
C. S. Jansen et al., An intra-tumoral niche maintains and differentiates stem-like CD8 T cells. Nature 576, 465-470 (2019).
D. Fantini et al., A Carcinogen-induced mouse model recapitulates the molecular alterations of human muscle invasive bladder cancer. Oncogene 37, 1911-1925 (2018).
D. I. Gabrilovich, S. Ostrand-Rosenberg, V. Bronte, Coordinated regulation of myeloid cells by tumours. Nat Rev Immunol 12, 253-268 (2012).
D. L. Stenoien et al., Polyglutamine-expanded androgen receptors form aggregates that sequester heat shock proteins, proteasome components and SRC-1, and are suppressed by the HDJ-2 chaperone. Hum Mol Genet 8, 731-741 (1999).
D. S. Thommen et al., A transcriptionally and functionally distinct PD-1 (+) CD8 (+) T cell pool with predictive potential in non-small-cell lung cancer treated with PD-1 blockade. Nat Med 24, 994-1004 (2018).
D. S. Thommen, T. N. Schumacher, T Cell Dysfunction in Cancer. Cancer Cell 33, 547-562 (2018).
D. T. Utzschneider et al., T Cell Factor 1-Expressing Memory-like CD8 (+) T Cells Sustain the Immune Response to Chronic Viral Infections. Immunity 45, 415-427 (2016).
D. V. Sawant et al., Adaptive plasticity of IL-10 (+) and IL-35 (+) Treg cells cooperatively promotes tumor T cell exhaustion. Nat Immunol 20, 724-735 (2019).
D. Y. Oh et al., Intratumoral CD4 (+) T Cells Mediate Anti-tumor Cytotoxicity in Human Bladder Cancer. Cell 181, 1612-1625 e1613 (2020).
E. E. Morales et al., Finasteride Reduces Risk of Bladder Cancer in a Large Prospective Screening Study. Eur Urol 69, 407-410 (2016).
E. J. Wherry, T cell exhaustion. Nat Immunol 12, 492-499 (2011).
E. L. Watts et al., Prospective analyses of testosterone and sex hormone-binding globulin with the risk of 19 types of cancer in men and postmenopausal women in UK Biobank. Int J Cancer 149, 573-584 (2021).
F. Bray et al., Global cancer statistics 2018: GLOBOCAN estimates of incidence and mortality worldwide for 36 cancers in 185 countries. CA Cancer J Clin 68, 394-424 (2018).
F. Conforti et al., Cancer immunotherapy efficacy and patients' sex: a systematic review and meta-analysis. Lancet Oncol 19, 737-746 (2018).
H. Kwon et al., Distinct CD8<sup>+</sup> T Cell Programming in the Tumor Microenvironment Contributes to Sex Bias in Bladder Cancer Outcome. bioRxiv, 2020.2004.2013.039735 (2020).
H. Kwon, C. Y. Lin, D. Chung, X. Li, Z. Li, Sex as a predictor of response to cancer immunotherapy. Lancet Oncol 19, e379 (2018).
H. T. Kissick et al., Androgens alter T-cell immunity by inhibiting T-helper 1 differentiation. Proc Natl Acad Sci USA 111, 9887-9892 (2014).
I. C. Summerhayes, L. M. Franks, Effects of donor age on neoplastic transformation of adult mouse bladder epithelium in vitro. J Natl Cancer Inst 62, 1017-1023 (1979).
I. Siddiqui et al., Intratumoral Tcf1 (+) PD-1 (+) CD8 (+) T Cells with Stem-like Properties Promote Tumor Control in Response to Vaccination and Checkpoint Blockade Immunotherapy. Immunity 50, 195-211 e110 (2019).
I. V. Kulakovskiy et al., HOCOMOCO: towards a complete collection of transcription factor binding models for human and mouse via large-scale ChIP-Seq analysis. Nucleic Acids Res 46, D252-D259 (2018).
J. Brummelman et al., High-dimensional single cell analysis identifies stem-like cytotoxic CD8 (+) T cells infiltrating human tumors. J Exp Med 215, 2520-2535 (2018).
J. R. Olsen et al., Context dependent regulatory patterns of the androgen receptor and androgen receptor target genes. BMC Cancer 16, 377 (2016).
J. S. Bertram, A. W. Craig, Specific induction of bladder cancer in mice by butyl-(4-hydroxybutyl)-nitrosamine and the effects of hormonal modifications on the sex difference in response. Eur J Cancer 8, 587-594 (1972).
J. S. Sutherland et al., Activation of thymic regeneration in mice and humans following androgen blockade. J Immunol 175, 2741-2753 (2005).
J. S. Yi, M. A. Cox, A. J. Zajac, T-cell exhaustion: characteristics, causes and conversion. Immunology 129, 474-481 (2010).
K. E. Yost et al., Clonal replacement of tumor-specific T cells following PD-1 blockade. Nat Med 25, 1251-1259 (2019).
K. Radojevic et al., Neonatal castration affects intrathymic kinetics of T-cell differentiation and the spleen T-cell level. J Endocrinol 192, 669-682 (2007).
K. Street et al., Slingshot: cell lineage and pseudotime inference for single-cell transcriptomics. BMC Genomics 19, 477 (2018).
L. McInnes, J. Healy, J. Melville, UMAP: Uniform Manifold Approximation and Projection for Dimension Reduction. arXiv e-prints. 2018.
M. A. Paley et al., Progenitor and terminal subsets of CD8⁺ T cells cooperate to contain chronic viral infection. Science 338, 1220-1225 (2012).
M. Ahmadzadeh et al., Tumor antigen-specific CD8 T cells infiltrating the tumor express high levels of PD-1 and are functionally impaired. Blood 114, 1537-1544 (2009).
M. Andreatta, S. J. Carmona, UCell: Robust and scalable single-cell gene signature scoring. Comput Struct Biotechnol J 19, 3796-3798 (2021).
M. Mercader et al., T cell infiltration of the prostate induced by androgen withdrawal in patients with prostate cancer. Proc Natl Acad Sci USA 98, 14565-14570 (2001).
M. S. Arredouani et al., Androgen ablation augments human HLA2.1-restricted T cell responses to PSA self-antigen in transgenic mice. Prostate 70, 1002-1011 (2010).
M. Sade-Feldman et al., Defining T Cell States Associated with Response to Checkpoint Immunotherapy in Melanoma. Cell 175, 998-1013 e1020 (2018).
M. W. LaFleur et al., PTPN2 regulates the generation of exhausted CD8 (+) T cell subpopulations and restrains tumor immunity. Nat Immunol 20, 1335-1347 (2019).
O. Khan et al., TOX transcriptionally and epigenetically programs CD8 (+) T cell exhaustion. Nature 571, 211-218 (2019).
P. Hartge et al., Unexplained excess risk of bladder cancer in men. J Natl Cancer Inst 82, 1636-1640 (1990).
P. L. Collins et al., DNA double-strand breaks induce H2Ax phosphorylation domains in a contact-dependent manner. Nat Commun 11, 3158 (2020).
P. Shannon et al., Cytoscape: a software environment for integrated models of biomolecular interaction networks. Genome Res 13, 2498-2504 (2003).
R. He et al., Follicular CXCR5-expressing CD8 (+) T cells curtail chronic viral infection. Nature 537, 412-428 (2016).
R. T. Abraham, A. Weiss, Jurkat T cells and development of the T-cell receptor signalling paradigm. Nat Rev Immunol 4, 301-308 (2004).
R. Zhao et al., A GPR174-CCL21 module imparts sexual dimorphism to humoral immunity. Nature 577, 416-420 (2020).
S. D. Blackburn, H. Shin, G. J. Freeman, E. J. Wherry, Selective expansion of a subset of exhausted CD8 T cells by alphaPD-L1 blockade. Proc Natl Acad Sci USA 105, 15016-15021 (2008).
S. Gupta, J. A. Stamatoyannopoulos, T. L. Bailey, W. S. Noble, Quantifying similarity between motifs. Genome Biol 8, R24 (2007).
S. Heinz et al., Simple combinations of lineage-determining transcription factors prime cis-regulatory elements required for macrophage and B cell identities. Mol Cell 38, 576-589 (2010).
S. J. Im et al., Defining CD8⁺ T cells that provide the proliferative burst after PD-1 therapy. Nature 537, 417-421 (2016).
S. Kaneko, X. Li, X chromosome protects against bladder cancer in females via a KDM6A-
S. Kurtulus et al., Checkpoint Blockade Immunotherapy Induces Dynamic Changes in PD-1 (−) CD8 (+) Tumor-Infiltrating T Cells. Immunity 50, 181-194 e186 (2019).
S. L. Klein, K. L. Flanagan, Sex differences in immune responses. Nat Rev Immunol 16, 626-638 (2016).
S. T. Gal-Oz et al., ImmGen report: sexual dimorphism in the immune system transcriptome. Nat Commun 10, 4295 (2019).
S. Van Gassen et al., FlowSOM: Using self-organizing maps for visualization and interpretation of cytometry data. Cytometry A 87, 636-645 (2015).
S. White-Gilbertson, M. Davis, C. Voelkel-Johnson, L. M. Kasman, Sex differences in the MB49 syngeneic, murine model of bladder cancer. Bladder (San Franc) 3, (2016).
T. L. Bailey, DREME: motif discovery in transcription factor ChIP-seq data. Bioinformatics 27, 1653-1659 (2011).
T. Le Bourgeois et al., Targeting T Cell Metabolism for Improvement of Cancer Immunotherapy. Front Oncol 8, 237 (2018).
T. Okazaki, S. Chikuma, Y. Iwai, S. Fagarasan, T. Honjo, A rheostat for immune responses: the unique properties of PD-1 and their advantages for clinical application. Nat Immunol 14, 1212-1218 (2013).
T. Wu et al., The TCF1-Bc16 axis counteracts type I interferon to repress exhaustion and maintain T cell stemness. Sci Immunol 1, (2016).
V. T. Fabris et al., Cytogenetic characterization of the murine bladder cancer model MB49 and the derived invasive line MB49-I. Cancer Genet 205, 168-176 (2012).
W. E. Naugler et al., Gender disparity in liver cancer due to sex differences in MyD88-dependent IL-6 production. Science 317, 121-124 (2007).
X. Guan et al., Androgen receptor activity in T cells limits checkpoint blockade efficacy. Nature, (2022).
X. Guo et al., Global characterization of T cells in non-small-cell lung cancer by single-cell sequencing. Nat Med 24, 978-985 (2018).
X. Qiu et al., Single-cell mRNA quantification and differential analysis with Census. Nat Methods 14, 309-315 (2017).
Y. Hao et al., Integrated analysis of multimodal single-cell data. Cell 184, 3573-3587 e3529 (2021).
Y. Yuan et al., Comprehensive Characterization of Molecular Differences in Cancer between Male and Female Patients. Cancer Cell 29, 711-722 (2016).
Y. Zhang et al., Model-based analysis of ChIP-Seq (MACS). Genome Biol 9, R137 (2008).
Z. Chen et al., Agonist and antagonist switch DNA motifs recognized by human androgen receptor in prostate cancer. EMBO J 34, 502-516 (2015).
Z. Chen et al., TCF-1-Centered Transcriptional Network Drives an Effector versus Exhausted CD8 T Cell-Fate Decision. Immunity 51, 840-855 e845 (2019).
Z. He, W. Kosinska, Z. L. Zhao, X. R. Wu, J. B. Guttenplan, Tissue-specific mutagenesis by N-butyl-N-(4-hydroxybutyl) nitrosamine as the basis for urothelial carcinogenesis. Mutat Res 742, 92-95 (2012).
Z. Li, G. Tuteja, J. Schug, K. H. Kaestner, Foxa1 and Foxa2 are essential for sexual dimorphism in liver cancer. Cell 148, 72-83 (2012).

G. Sequences
SEQ ID NO: 1 Ar: forward primer
TCCAAGACCTATCGAGGAGCG
SEQ ID NO: 2 Ar: reverse primer
GTGGGCTTGAGGAGAACCAT
SEQ ID NO: 3 Tcf7 forward primer
CCACTCTACGAACATTTCAGCA
SEQ ID NO: 4 Tcf7 reverse primer
ACTGGGCCAGCTCACAGTA
SEQ ID NO: 5 Haver2: forward primer
TCAGGTCTTACCCTCAACTGTG
SEQ ID NO: 6 Haver2: reverse primer
GGCATTCTTACCAACCTCAAACA
SEQ ID NO: 7 Isg15: forward primer
GGTGTCCGTGACTAACTCCAT
SEQ ID NO: 8 Isg15: reverse primer
CTGTACCACTAGCATCACTGTG
SEQ ID NO: 9 β-actin: forward primer
AGCTGAGAGGGAAATCGTGC
SEQ ID NO: 10 9 β-actin: reverse primer
TCCAGGGAGGAAGAGGATGC
SEQ ID NO: 11 Sry (Set 1): forward primer
TTGTCTAGAGAGCATGGAGGGCCATGTCAA
SEQ ID NO: 12 Sry (Set 1): reverse primer
CCACTCCTCTGTGACACTTTAGCCCTCCGA
SEQ ID NO: 13 Sry (Set 2): forward primer
TGGGACTGGTGACAATTGTC
SEQ ID NO: 14 Sry (Set 2): reverse primer
GAGTACAGGTGTGCAGCTCT
SEQ ID NO: 15
AGAACAnnnAGTACT
SEQ ID NO: 16
AGAACAnnnAGTGCT
SEQ ID NO: 17 hDNase NC2-chr13: forward primer
GGCACATACCCATTTGTCCCAACA
SEQ ID NO: 18 hDNase NC2-chr13: reverse primer
GACAAAGGTGTCAAGAACACACAATGGG
SEQ ID NO: 19 hTcf7 hAR_3: forward primer
GTACGAGCACAGCCTCAAG
SEQ ID NO: 20 hTcf7 hAR_3: reverse primer
GCCGTGGTGTGAACTGTAT
SEQ ID NO: 21 hARE1 binding site
CGGGCTGCAGGTTCT
SEQ ID NO: 22 hARE2 binding site
AACGCTGCCGGTTCC
SEQ ID NO: 23 hARE3 binding site
AGCACAGGGCGCATT
SEQ ID NO: 24 hARE4 binding site
GGAAGAGCGAGCCCT
SEQ ID NO: 25 mARE1 binding site
TGGACAGCAGGTTCT
SEQ ID NO: 26 mARE2 binding site
ACAACAGGCAGGAGC
SEQ ID NO: 27 mARE3 binding site
AGGAAATGGAGTTCC
SEQ ID NO: 28 mARE4 binding site
TGCCTTTGATGTTCC
SEQ ID NO: 29 mARE5 binding site
GGACCTGGCAGAGCT

What is claimed is:

1. A method for promoting/enhancing differentiation of T cells to an effector state in a subject comprising administering to the subject an inhibitor of androgen receptor (AR) directed to an AR on the T cells of the subject.

2. The method of claim 1, wherein the T cell is a CD8+ T cells.

3. The method of claim 1, wherein the inhibitor of AR comprises genetic manipulation or androgen deprivation therapy.

4. The method of claim 3, wherein the genetic manipulation involves CRISPR/Cas9-mediated knockout of the AR gene in the CD8+ T cells.

5. The method of claim 3, wherein the androgen deprivation therapy comprises administration of Luteinizing Hormone-Releasing Hormone (LHRH) agonists comprising but not limited to leuprolide, goserelin, and triptorelin.

6. The method of claim 3, wherein the androgen deprivation therapy comprises administration of Androgen Inhibitors comprising but not limited to bicalutamide, enzalutamide, apalutamide, flutamide, darolutamide, nilutamide, abiraterone, degarelix, relugolix, leuprolide, or goserelin.

7. The method of claim 3, wherein the androgen deprivation therapy comprises administration of Androgen Synthesis Inhibitors comprising but not limited to abiraterone.

8. A method for rescuing terminally exhausted T cells and/or progenitor exhausted T cells and driving said T cells to an effector-like state in a subject with a cancer comprises comprising administering to the subject an inhibitor of androgen receptor (AR).

9. The method of claim 8, further comprising monitoring the differentiation state of CD8+ T cells in the subject by flow cytometry analysis of T cell markers including GZMB, TOX, TCF1, and PD-1.

10. The method of claim 8, wherein the cancer comprises T cell lymphoma, mycosis fungoides, Hodgkin's Disease, acute myeloid leukemia (AML), chronic myeloid leukemia (CML), brain cancer, nervous system cancer, head and neck cancer, renal cancer, lung cancers, neuroblastoma/glioblastoma, ovarian cancer, pancreatic cancer, skin cancer, hepatic cancer, melanoma, squamous cell carcinomas of the mouth, throat, larynx, and lung, cervical cancer, breast cancer, genitourinary cancer, esophageal carcinoma, hematopoietic cancers, testicular cancer, colon cancer, or rectal cancer.

11. The method of claim 10, wherein the cancer comprises prostate cancer, bladder cancer, and other cancers exhibiting AR-mediated immune modulation.

12. The method of claim 8, wherein the inhibitor of AR comprises genetic manipulation or androgen deprivation therapy.

13. The method of claim 12, wherein the genetic manipulation involves CRISPR/Cas9-mediated knockout of the AR gene in the CD8+ T cells.

14. The method of claim 12, wherein the androgen deprivation therapy comprises administration of Luteinizing Hormone-Releasing Hormone (LHRH) agonists comprising but not limited to leuprolide, goserelin, and triptorelin.

15. The method of claim 12, wherein the androgen deprivation therapy comprises administration of Androgen Inhibitors comprising but not limited to bicalutamide, enzalutamide, apalutamide, flutamide, darolutamide, nilutamide, abiraterone, degarelix, relugolix, leuprolide, or goserelin.

16. The method of claim 12, wherein the androgen deprivation therapy comprises administration of Androgen Synthesis Inhibitors comprising but not limited to abiraterone.

17. A method for treating a cancer in a subject, comprising:
administering to the subject with the cancer a therapeutically effective amount of an androgen receptor (AR) inhibitor;
wherein the AR inhibitor promotes differentiation of CD8+ T cells from a terminally exhausted state or progenitor exhausted state to a granzyme B+ (GZMB+) effector-like state; and
wherein the cancer is characterized by AR-dependent modulation of immune responses.

18. The method of claim 17, wherein the cancer comprises T cell lymphoma, mycosis fungoides, Hodgkin's Disease, acute myeloid leukemia (AML), chronic myeloid leukemia (CML), brain cancer, nervous system cancer, head and neck cancer, renal cancer, lung cancers, neuroblastoma/glioblastoma, ovarian cancer, pancreatic cancer, skin cancer, hepatic cancer, melanoma, squamous cell carcinomas of the mouth, throat, larynx, and lung, cervical cancer, breast cancer, genitourinary cancer, esophageal carcinoma, hematopoietic cancers, testicular cancer, colon cancer, or rectal cancer.

19. The method of claim 18, wherein the cancer comprises prostate cancer, bladder cancer, and other cancers exhibiting AR-mediated immune modulation.

20. The method of any of claim 17, wherein the modulation of immune responses further comprises monitoring differentiation state of CD8+ T cells in the subject by flow cytometry analysis of T cell markers including GZMB, TOX, TCF1, and PD-1.

21. The method of claim 17, wherein the AR inhibitor comprises genetic manipulation or androgen deprivation therapy.

22. The method of claim 21, wherein the genetic manipulation involves CRISPR/Cas9-mediated knockout of the AR gene in the CD8+ T cells.

23. The method of claim 21, wherein the androgen deprivation therapy comprises administration of Luteinizing Hormone-Releasing Hormone (LHRH) agonists comprising but not limited to leuprolide, goserelin, and triptorelin.

24. The method of claim 21, wherein the androgen deprivation therapy comprises administration of Androgen Inhibitors comprising but not limited to bicalutamide, enzalutamide, apalutamide, flutamide, darolutamide, nilutamide, abiraterone, degarelix, relugolix, leuprolide, or goserelin.

25. The method of claim 21, wherein the androgen deprivation therapy comprises administration of Androgen Synthesis Inhibitors comprising but not limited to abiraterone.

26. The method of claim 17, further comprising subjecting the subject to conventional cancer therapy, wherein the conventional cancer therapy comprises including but not limited to surgery, chemotherapy, or radiation therapy.

27. A method for treating a subject with a cancer comprising a) obtaining a biological sample from the subject;
b) measuring the level of androgen receptor (AR) expression or activity in CD8+ T cells; wherein an increase in the level of AR expression or activity relative to a control indicates suitability for AR inhibitor therapy; and
c) administering an AR inhibitor when the subject has an increase in the level of AR expression or activity.

28. The method of claim 27, wherein the AR inhibitor comprises genetic manipulation or androgen deprivation therapy

29. The method of claim 28, wherein the genetic manipulation involves CRISPR/Cas9-mediated knockout of the AR gene in the CD8+ T cells.

30. The method of claim 28, wherein the androgen deprivation therapy comprises administration of Luteinizing Hormone-Releasing Hormone (LHRH) agonists comprising but not limited to leuprolide, goserelin, and triptorelin.

31. The method of claim 28, wherein the androgen deprivation therapy comprises administration of Androgen Inhibitors comprising but not limited to bicalutamide, enzalutamide, apalutamide, flutamide, darolutamide, nilutamide, abiraterone, degarelix, relugolix, leuprolide, or goserelin.

32. The method of claim 28, wherein the androgen deprivation therapy comprises administration of Androgen Synthesis Inhibitors comprising but not limited to abiraterone.

33. The method of claim 27, wherein the cancer comprises T cell lymphoma, mycosis fungoides, Hodgkin's Disease, acute myeloid leukemia (AML), chronic myeloid leukemia (CML), brain cancer, nervous system cancer, head and neck cancer, renal cancer, lung cancers, neuroblastoma/glioblastoma, ovarian cancer, pancreatic cancer, skin cancer, hepatic cancer, melanoma, squamous cell carcinomas of the mouth, throat, larynx, and lung, cervical cancer, breast cancer, genitourinary cancer, esophageal carcinoma, hematopoietic cancers, testicular cancer, colon cancer, or rectal cancer.

34. The method of claim 33, wherein the cancer comprises prostate cancer, bladder cancer, and other cancers exhibiting AR-mediated immune modulation.
